(19) United States
(12) Patent Application Publication
(10) Pub. No.: US 2024/0125773 A1
(43) Pub. Date: Apr. 18, 2024

(54) COMPOSITIONS AND METHODS FOR PREVENTING NON-SPECIFIC INTERACTIONS BETWEEN POLYMER DYES-ANTIBODY CONJUGATES

(71) Applicant: BECKMAN COULTER, INC., Brea, CA (US)

(72) Inventors: Frederic MONSONIS, Marseille (FR); Boi Hoa SAN, Miami, FL (US); Arunkumar EASWARAN, Miami, FL (US); Massimiliano TOMASULO, Miami, FL (US); Sergei GULNIK, San Jose, CA (US); Rajesh VENKATESH, Bangalore (IN); Shiva Ranjini SRINIVASAN, Kumbakonam (IN)

(21) Appl. No.: 18/264,363

(22) PCT Filed: Feb. 4, 2022
(86) PCT No.: PCT/US2022/015296

§ 371 Date: Aug. 4, 2023

Related U.S. Application Data
(60) Provisional application No. 63/146,498, filed on Feb. 5, 2021.

(51) Int. Cl. G01N 33/533; G01N 33/53

(52) U.S. Cl. CPC G01N 33/533; G01N 33/5306

(57) ABSTRACT

The disclosure relates to compositions comprising a non-fluorescent component of a polymer dye such as a monomeric component of a polymer dye, a photo-bleached polymer dye, and/or a polymer dye comprising a quenching moiety, for reducing non-specific interactions of polymer dye conjugates, for example, in Flow Cytometric Analysis of a biological sample. Methods for using such compositions, and kits comprising such compositions are also provided.

### CROSS REFERENCES TO RELATED APPLICATIONS

This application is being filed 4 Feb. 2022 as a PCT International Patent Application and claims the benefit of and priority to U.S. Provisional Application No. 63/146,498, filed 5 Feb. 2021, which is incorporated by reference in its entirety.

### BACKGROUND

Polymer dye conjugates are bright and provide excellent performance that can be utilized in multi-color flow cytometry assays. In general, polymer dye conjugates exhibit high brightness due to their unique and complex structure. But that same unique and complex structure also leads to some significant limitations.

Polymer dyes are hydrophobic and have large apparent molecular weights which makes them prone to aggregation in aqueous buffer. Consequently, when polymer dye conjugated to antibodies, the resulting conjugates may also have propensity to interact with each other and/or with other polymer dye conjugates present in the same sample. When more than one polymer dye conjugate is used for staining the same sample, non-specific interaction between the polymer dyes may occur which can result in under-compensation of data.

A specialized staining buffer that can eliminate non-specific interaction between polymer dye conjugates is highly desirable.

### SUMMARY

Because of their nature, polymer dye conjugates can interact with one another, leading to non-specific binding. The instant disclosure provides compositions for decreasing or preventing non-specific interactions between polymer dye conjugates.

The disclosure provides a composition comprising one or more non-fluorescent components of a polymer dye and a buffer capable of reducing non-specific interactions between dye conjugates. The composition optionally may further comprise a nonionic surfactant. The composition optionally may further comprise a protein stabilizer. The composition optionally may further comprise a preservative.

The disclosure provides a staining buffer composition comprising one or more non-fluorescent components of a polymer dye, a nonionic surfactant and a buffer, wherein the composition is capable of reducing non-specific binding such as polymer-polymer interactions between one or more fluorescent polymer dye conjugates.

The non-fluorescent component of the polymer dye may be one or more of a monomeric component of a polymer dye, a photo-bleached polymer dye, and a polymer dye comprising a quenching moiety such as a quenched polymer dye.

The disclosure provides a composition comprising a monomeric component of a polymer dye in a buffer.

The disclosure provides a composition comprising a photo-bleached dye in a buffer.

The disclosure provides a composition comprising a quenched polymer dye in a buffer.

The disclosure provides a composition comprising a monomeric component of a polymer dye and a quenched polymer dye in a buffer.

The disclosure provides a composition comprising a monomeric component of a polymer dye and a photo-bleached dye in a buffer.

The compositions of the disclosure may optionally further include a protein stabilizer, a nonionic surfactant, and/or a preservative. For example, a working concentration composition (1×) of the disclosure may further include protein stabilizer at 0.1-10 mg/mL, 0.5-5 mg/mL, 1-3 mg/mL, or about 2 mg/mL.

A concentrated staining buffer composition (10×) of the disclosure may further include a protein stabilizer at 1-100 mg/mL, 2-50 mg/mL, 5-40 mg/mL, 10-30 mg/mL, or about 20 mg/mL. In some embodiments, the protein stabilizer is present at 0.1-100 mg/mL, 0.2-50 mg/mL, 1-20 mg/mL, or 1-10 mg/mL.

A working concentration composition (1×) of the disclosure may include a nonionic surfactant at 0.01-10%, 0.01-4%, 0.01-2%, 0.01-0.4% or about 0.02% (wt/vol).

A concentrated staining buffer composition (10×) of the disclosure may include a nonionic surfactant at 0.1-40%, 0.5-20%, 1-10%, or about 5% (wt/vol).

The composition of the disclosure may optionally further include a preservative at 0.01-0.5%, 0.03-0.3%, or 0.05-0.2%, or about 0.1% (wt/vol).

For example, the disclosure provides a working concentration staining buffer composition (1×) comprising 0.3-1.5 mg/mL, 0.5-1.2 mg/mL, or about 1 mg/mL of a quenched polymer dye in a biological buffer such as a PBS buffer comprising 0.01-10%, or 0.1-4% (wt/vol) of a nonionic surfactant, and optionally 0.1-10 mg/mL, or 0.2-5 mg/mL of a protein stabilizer.

The disclosure provides a concentrated staining buffer composition (10×) comprising 3-15 mg/mL, 5-12 mg/mL, or about 10 mg/mL of a quenched polymer dye in a biological buffer such as a PBS buffer comprising 0.1-40%, or 1-10% (wt/vol) of a nonionic surfactant, and optionally 1-100 mg/mL, or 2-50 mg/mL of a protein stabilizer. This composition was found to significantly decrease non-specific polymer dye conjugate interactions in a multi-color dye conjugate panel. This was evidenced in FCA flow cytometry analysis of a stained and lysed blood sample when compared to the same sample without the quenched polymer dye.

The disclosure provides a working concentration staining buffer composition (1×) comprising 20-40 mg/mL, or about 30 mg/mL of a monomeric component of a polymer dye; 0.2-0.8 mg/mL, or 0.3-0.7 mg/mL of a quenched polymer dye; and 0.01-10%, or 0.1-4% of a nonionic surfactant, in a biological buffer. The working concentration composition (1×) may further comprise 0.1-10 mg/mL, 0.5-5 mg/mL of a protein stabilizer.

The disclosure provides a concentrated staining buffer composition (10×) comprising 200-400 mg/mL, or about 300 mg/mL of a monomeric component of a polymer dye; 2-8 mg/mL, or 3-7 mg/mL quenched polymer dye; and 0.1-40%, or 1-20% of a nonionic surfactant, in a biological buffer. The concentrated composition (10×) may further comprise 1-100 mg/mL, or 5-50 mg/mL of a protein stabilizer.

This composition was found to significantly decrease non-specific polymer dye conjugate interactions in a multi-color dye conjugate panel. This was evidenced in a Flow Cytometric Analysis (FCA) of a stained and lysed whole blood sample when compared to the same sample without the monomeric component and without the quenched polymer dye.

The disclosure provides a working concentration staining buffer composition (1×) comprising 20-40 mg/mL, or about 30 mg/mL of a monomeric component of a polymer dye; 0.2-0.8 mg/mL, 0.3-0.7 mg/mL, or about 0.5 mg/mL of a photo-bleached polymer dye; and 0.01-10%, or 0.1-4% of a nonionic surfactant, in a biological buffer. The working concentration composition (1×) may further comprise 0.1-10 mg/mL, or 0.5-5 mg/mL of a protein stabilizer.

The disclosure provides a concentrated staining buffer composition (10×) comprising 200-400 mg/mL, or about 300 mg/mL of a monomeric component of a polymer dye, 2-8 mg/mL, 3-7 mg/mL, or about 5 mg/mL of a photo-bleached polymer dye, and 0.1-40%, or 1-20% of a nonionic surfactant, in a biological buffer. The concentrated composition (10×) may further comprise 1-100 mg/mL, or 2-50 mg/mL of a protein stabilizer.

This composition was found to substantially decrease non-specific polymer dye conjugate interactions in a multi-color dye conjugate panel. This was evidenced in a FCA analysis of a stained and lysed whole blood sample when compared to the same sample without the monomeric component and without the photo-bleached polymer dye.

A composition is provided for use with at least one fluorescent polymer dye conjugated to a binding partner for use in staining a biological sample, the composition comprising a nonionic surfactant; and a biological buffer; wherein the composition reduces non-specific binding of the at least one fluorescent polymer dye conjugate, when compared to the at least one fluorescent polymer dye conjugate in the absence of the composition. The nonionic surfactant may be a poloxamer.

A method is provided for detecting an analyte in a sample comprising: adding at least one, or at least two, polymer dye conjugates to a staining buffer composition according to the disclosure to form a polymer dye conjugate composition; contacting a biological sample that is suspected of containing an analyte with the polymer dye conjugate composition to form a fluorescent polymer dye conjugate complex with the analyte; applying a light source to the sample that can excite the at least one, or at least two, fluorescent polymer dye conjugate complexes; and detecting light emitted from the fluorescent polymer dye conjugate complex. The detecting light may comprise analyzing by flow cytometry to obtain a first flow cytometry plot, wherein the first flow cytometry plot exhibits one or more of the group consisting of: decreased non-specific interaction of polymer dye conjugates; and decreased aggregation of polymer dye conjugates, when compared to a second flow cytometry plot obtained comprising contacting the biological sample with a composition without the nonionic surfactant and without the non-fluorescent component of the first polymer dye.

The biological sample may be a blood, bone marrow, spleen cells, lymph cells, bone marrow aspirates, urine, serum, saliva, cerebral spinal fluid, urine, amniotic fluid, interstitial fluid, feces, mucus, tissue sample, or cell culture sample. The biological sample may be a whole blood sample.

The light from the light source may have a wavelength within a range of between about 340 nm and about 800 nm, or a wavelength within a range of between about 340 nm and about 450 nm.

The disclosure provides a kit comprising a staining buffer composition according to the disclosure, wherein the kit comprises separate containers comprising the one or more non-fluorescent component of a first polymer dye; and at least one fluorescent polymer dye conjugate. The staining buffer composition may include a nonionic surfactant in the same container comprising the one or more non-fluorescent component. The staining buffer composition may comprise two or more of the non-fluorescent components of a first polymer dye and optionally a nonionic surfactant in one container; and the at least one fluorescent polymer dye conjugate in a separate container.

### DESCRIPTION OF THE DRAWINGS

FIG. 1 shows FCA dot blot comparison of test staining buffer using a mixture of three SuperNova (SNv) dye antibody conjugates CD56-SNv428, CD20-SNv605, and CD4-SNv786 with or without pre-addition of test staining buffer with stained and lysed whole blood. Gating is on lymphocytes (LY). As shown in upper three panels, abnormal staining can occur in absence of the test staining buffer and data can appear under-compensated. In the presence of test staining buffer comprising a photo-bleached polymer and monomeric component of a polymer dye, as shown in lower three panels, substantially reduced non-specific polymer dye conjugate interactions occur.

FIG. 2A shows FCA dot plots of stained and lysed whole blood sample with a mixture of three SuperNova violet polymer dye conjugates including CD56-SNv428+CD19-SNv605+CD4-SNv786 without staining buffer components. The positive and negative cell populations in the dot plots are not aligned and look tilted, indicative of non-specific polymer dye interactions.

FIG. 2B shows FCA dot plots of stained and lysed whole blood sample with a mixture of three SuperNova violet polymer dye conjugates including CD56-SNv428+CD19-SNv605+CD4-SNv786 with a commercial comparative staining buffer. Compared to FIG. 2A, somewhat reduced non-specific interactions are apparent.

FIG. 2C shows FCA dot plots of stained and lysed whole blood sample with a mixture of three SuperNova violet polymer dye conjugates including CD56-SNv428+CD19-SNv605+CD4-SNv786 and a Test staining buffer according to the disclosure comprising monomer A and photo-bleached dye 428 in a biological buffer with a poloxamer nonionic surfactant. The test staining buffer composition exhibited reduced spillover when compared to FIG. 2A, and somewhat reduced non-specific polymer-polymer interactions compared to prior art comparative buffer in FIG. 2B.

FIG. 3 shows a graph of fluorescent profiles in wavelength vs. AFU of Quenched polymers. Polymer 1 Dabcyl (QY 0.01), polymer 2 Dabcyl (QY 0.005), polymer 2 DY425Q (QY 0.01), polymer 3 Dabcyl (QY 0.005), polymer 3 Dabcyl plus (QY 0.015), and polymer 3 DY425Q (QY 0.009). FIG. 3 inset shows a graph of representative parent fluorescent polymer (Polymer 3 QY 0.54) before and after conjugation to the quenching moiety to obtain the quenched polymer which exhibits substantially reduced fluorescence QY when excited by a 405 nm laser. QY refers to quantum yield.

FIG. 4 shows (upper row, left to right) FCA dot plots of stained and lysed whole blood samples after staining with a mixture of two polymer dye conjugates CD4-UV excitable polymer dye (CD4-UVEPD) and CD20-Violet excitable polymer dye (CD20-VEPD) (both Beckman Coulter Life Sciences, upper left panel), and a mixture of two polymer dye conjugates CD4-BUV395 (BD Biosciences) and CD20-VEPD (Beckman Coulter Life Sciences, upper right panel) when gated on lymphocytes, without staining buffer additive quenched polymer. When two different dye conjugates are mixed without quenched polymer, the dot plots exhibit non-specific interaction and associated spillover. Addition of quenched polymer according to the disclosure (Quenched Polymer 2-DYQ 425) during staining with CD4-UVEPD and CD20-VEPD (bottom left panel), or BUV395-CD4 and CD20-VEPD (bottom right panel), reduced non-specific interactions and spillover

FIG. 5 shows (upper row, left to right) FCA dot plots of stained and lysed whole blood samples after staining with a mixture of the two polymer dye conjugates CD4-UV excitable polymer dye (CD4-UVEPD) and CD20-Violet excitable polymer dye (CD20-VEPD) (both Beckman Coulter Life Sciences, upper left panel), and a mixture of two polymer dye conjugates CD4-BUV395 (BD Biosciences) and CD20-VEPD (Beckman Coulter Life Sciences, upper right panel) when gated on lymphocytes, without staining buffer additive quenched polymer. When two different dye conjugates are mixed without quenched polymer, the dot plots exhibit non-specific interaction and associated spillover. Addition of quenched polymer according to the disclosure (Quenched Polymer 2-Dabcyl) during staining with CD4-UVEPD and CD20-VEPD (bottom left panel), or BUV395-CD4 and CD20-VEPD (bottom right panel), reduced non-specific interactions and spillover.

FIG. 6 shows two-dimensional FCA dot plots of SuperNova polymer dye conjugates CD56-SNv428/CD4-SNv786 in a sample prepared with a composition comprising ineffectively photo-bleached dye (left panel) resulting in undesirable spillover of the conjugates. In contrast, the right panel shows the effect of a composition according to the disclosure including a correctly photo-bleached dye having QY≤0.056 and <47 AFU at 10 ug/mL when excited with 405 nm laser. Spillover of conjugates is significantly reduced.

FIG. 7 shows two-dimensional FCA dot plots of two SuperNova polymer dye conjugates CD3-SNv428/CD19-SNv605 individually pre-formulated with or without Monomer A+Poly-Dabcyl additives before being added in the biological sample. Without additives (left panel) non-specific interactions are evident. In the presence of Monomer A +Poly-Dabcyl additives (right panel) non-specific interactions of polymer dye conjugates are substantially reduced.

FIG. 8 shows FCA dot plots of stained and lysed cells with a mixture of dye conjugates CD19-SNv428 (Beckman Coulter Life Sciences) and CD4-BV650 (BD Biosciences) in a blood sample without quenched polymer (upper left), with 1% PF-68 (upper right), with 10 ug quenched polymer 2-Dabcyl (bottom left), with 10 ug quenched polymer 2-Dabcyl and 1% PF-68 nonionic surfactant (bottom right). The test sample compositions with 10 ug quenched polymer 2-Dabcyl (MFI 2172) and 1% PF-68 (MFI 2400) exhibited decreased non-specific binding compared to controlled sample with no buffer (MFI 7804). The test sample composition with 10 ug quenched polymer 2-Dabcyl and 1% PF-68 (MFI 1327) exhibited improved decreased MFI, improved decreased non-specific binding, and improved decreased polymer-polymer interactions compared to controlled samples.

FIG. 9 shows FCA dot plots of stained and lysed cells with a mixture of CD4-BV650 (BD Biosciences) and CD19-SNv428 (Beckman Coulter Life Sciences) without buffer (upper panel), with 0.1% nonionic surfactant (lower left panel), 0.5% nonionic surfactant (lower middle panel), and 1% nonionic surfactant (wt/vol) (lower right panel). The presence of increasing concentration of nonionic surfactant (0.1-1% wt/vol) is associated with decreased spillover and non-specific interactions in the mixture as evidenced by improved separation compared to without nonionic surfactant.

FIG. 10 shows a graph of emission spectra over 415-700 nm and quantum yield of two non-fluorescent polymer dyes useful for decreasing non-specific interactions and spillover in FCA analysis in staining buffer compositions. Structures of DHP-pyrrole polymer (QY 0.043) and DHP-nitro capped polymer (QY 0.092) are also shown.

### DESCRIPTION

Reference will now be made in detail to certain embodiments of the disclosed subject matter, examples of which are illustrated in part in the accompanying drawings and Examples. While the disclosed subject matter will be described in conjunction with the enumerated claims, it will be understood that the exemplified subject matter is not intended to limit the claims to the disclosed subject matter.

Provided herein are compositions and methods for reducing non-specific interactions of polymer dye conjugates, for ex-ample, in a multi-color panel comprising a multiplicity of dye conjugates. The compositions and methods are useful for reducing polymer-polymer interactions, for example, that may result in increased spill over into other channels in flow cytometry.

The disclosure generally relates to staining buffers designed to reduce non-specific interactions between fluorescent polymer dye conjugates, compositions comprising such polymers, and methods for detecting analytes in a sample using compositions comprising fluorescent polymers conjugated to binding partners (e.g., a polymer dye conjugated to an antibody, which is referenced herein as “a polymer dye conjugate”). For example, compositions according to the disclosure include a liquid staining buffer comprising at least one, or at least two, non-fluorescent components of a polymer dye, a buffer, and optionally a nonionic surfactant. The non-fluorescent component of a polymer dye may be one or more of a monomeric component of a polymer dye, a photo-bleached polymer dye, and a polymer dye comprising a quenching moiety.

For example, the compositions described herein may include a monomeric component of a polymer dye and a photo-bleached polymer dye; a monomeric component of a polymer dye and a polymer dye comprising a quenching moiety; or a photo-bleached polymer dye and a non-ionic surfactant. The compositions according to the disclosure are capable of reducing non-specific polymer-polymer interactions between different polymer dye conjugates in a multi-color panel.

### Polymer Dyes

The staining buffer compositions of the disclosure may include one or more non-fluorescent components of a polymer dye. The non-fluorescent component may be a non-fluorescent polymer dye. The non-fluorescent component of a polymer dye may be a polymer dye comprising a quenching moiety. The non-fluorescent component of a polymer dye may be a photo-bleached polymer dye. The non-fluorescent component of a polymer dye may be a monomeric component of a polymer dye (“monomer”). In some embodiments, the non-fluorescent component of a polymer dye does not comprise a binding partner. In some embodiments, polymer dye also includes polymer tandem dye. In some embodiments, polymer dye also includes polymer dye conjugated to a functional moiety.

The compositions of the disclosure may be used with one or more fluorescent polymer dye conjugates. In some embodiments, polymer dye also includes polymer dye conjugated to a binding partner. In some embodiments, polymer dye also includes polymer dye conjugated to a functional moiety.

Fluorescent polymer dyes are particularly useful for analysis of chemical and biological target analytes. They are highly responsive optical reporters and efficient light absorbers, by virtue of the multiple chromophores they comprise.

The polymer dye can comprise any previously disclosed or commercially available fluorescent polymer dye. For example, the polymer dye can be any dye disclosed in Published PCT Appl. No. WO 20221013198; Published PCT Appl. No. WO 2017/180998; U.S. Application No. 2021/0047476; U.S. Application No. 2020/0190253; U.S. Application No. 2020/0048469; U.S. Application No. 2020/0147615; U.S. Application No. 2021/0108083; U.S. Application No. 2019/0194467; U.S. Application No, 2018/0364245; U.S. Application No. 2018/0224460; U.S. Pat. Nos. 11,034,840; 11,119,107; 10,962,546; 10,920,082; 10,001,475; 10,107,818; 10,228,375; 10,844,228; 10,604,657; 10,545,137B2; 10,533,092; 10,472,521; 10,240,000; 9,758,625; 9,719,998; 7,214,489; 9,012,643; 8,623,332; 8,431,416; 8,354,239; 8,575,303; 8,969,509, each of which are incorporated by reference as if fully set forth herein in their entirety. The polymer dye can have the structure of any water-soluble fluorescent polymer dye disclosed in Published US Appl. No. 2020/0190253 A1, which is incorporated by reference as if fully set forth herein in its entirety. The polymer dye conjugate can have the structure of any water-soluble fluorescent polymer dye disclosed in Published US Appl. No. 2019/0144601, which is Incorporated by reference as if fully set forth herein in its entirety.

The polymer dye can be any commercially available polymer dye. In some embodiments, the polymer dye can be excitable by, for example, ultraviolet (e.g., 351 nm, 355 nm, 375 nm, 334-364 nm, 351-356 nm), violet (e.g., 405 nm, 407 nm, 414 nm, 395-425 nm), blue (e.g., 436 nm, 458 nm), blue-green (e.g., 488 nm), green (e.g., 514 nm, 532 nm, 541 nm, 552 nm), yellow-green (e.g., 561 nm, 563 nm), yellow (e.g., 568 nm), red (e.g., 627-640 nm, 633 nm, 637 nm, 640 nm, 647 nm), and/or near infrared lasers (e.g., 673 nm, 750 nm, 780 nm, or in a range of from 660-800 nm). The polymer dye may comprise a polymer dye excitable by a violet laser. The polymer dye or polymer dye conjugate may comprise a polymer dye excitable by a violet laser at a wavelength from about 395 nm to about 425 nm, for example, 405 nm, 407 nm, or 414 nm. The polymer dye or polymer dye conjugate may comprise a violet laser (405 nm)-excitable polymer dye. In some embodiments, the polymer dye may be a non-fluorescent polymer dye.

In some embodiments, the polymer dye or polymer dye conjugate may comprise a SuperNova polymer dye (SN) (Beckman Coulter, Inc.). SuperNova polymer dyes are a new generation of polymer dyes useful for flow cytometry application. The polymer dye or polymer dye conjugate may comprise SNv428, SNv605 or SNv786. SNv428 has unique photo-physical properties leading to extremely bright conjugates when conjugated to antibodies or other binding partners. For example, SNv428 is a polymer dye optimally excited by the violet laser (e.g., 405 nm) with an excitation maximum of 414 nm, an emission peak of 428 nm, and can be detected using a 450/50 bandpass filter or equivalent.

SNv428 is one of the brightest dyes excitable by the violet laser, so it is particularly suited for assessing dimly expressed markers. SuperNova polymer dye conjugated with antibodies may include anti-CD19 antibody-SNv428, anti-CD22 antibody-SNv428, anti-CD25 antibody-SNv428, and anti-CD38 antibody-SNv428 antibody-polymeric dye conjugates.

SNv605 and SNv786 (Beckman Coulter, Inc.) are tandem polymer dyes, derived from the core SNv428. Both share the same absorbance characteristics, with maximum excitation at 414 nm. With SNv605 and SNv786 having emission peaks at 605 nm and 786 nm, respectively, they are optimally detected using the 610/20 and 780/60 nm bandpass filters of the flow cytometer. SNv605 and SNv786 may be conjugated, for example, with a binding partner such as an anti-CD19 antibody, anti-CD22 antibody, anti-CD25 antibody, or anti-CD38 antibody.

The polymer dye may comprise a polymer dye excitable by an ultra-violet (“UV”) laser. The polymer dye or polymer dye conjugate may comprise a polymer dye excitable by a UV laser at a wavelength of 320 nm to 380 nm, 340 nm to 360 nm, 345 nm to 356 nm, or less than or equal to 380 nm but greater than or equal to 320 nm. The polymer dye or polymer dye conjugate may comprise a UV-excitable polymer dye. The UV-excitable polymer dye or polymer dye conjugate may emit light typically at a wavelength of 380 nm to 430 nm, 406 nm to 415 nm, or less than or equal to 430 nm but greater than or equal to 380 nm.

The polymer dye can comprise a Brilliant Violet™ dye (BioLegend®/Sirigen Group Ltd.), such as Brilliant Violet 421™ (excitation max. 405 nm, emission max. 421 nm, 450/50 filter), Brilliant Violet 510™ (excitation max 405 nm, emission max 510 nm, 510/50 filter), Brilliant Violet 570™ (excitation max 405 nm, emission max 570 nm, 585/42 filter), Brilliant Violet 605™ (excitation max 405 nm, emission max 603 nm, 610/20 filter), Brilliant Violet 650™ (excitation max 405 nm, emission max 645 nm, 660/20 filter), Brilliant Violet 711™ (excitation max 405 nm, emission max 711 nm, 710/50 filter), Brilliant Violet 750™ (excitation max 405 nm, emission max 750 nm, 780/60 filter), Brilliant Violet 785™ (excitation max 405 nm, emission max 785 nm, 780/60 filter).

The polymer dye or polymer dye conjugate may comprise a BD Horizon Brilliant™ Violet (“BV”) polymer dye (Becton, Dickinson and Co., BD Life Sciences). The polymer dye may be a BD Horizon Brilliant™ BV421 (450/40 or 431/28 filter), BV480 (525/40 filter), BV510 (525/40 filter), BV605 (610/20 filter), BV650 (660/20 filter), BV711 (710/50 filter), BV786 (786/60 filter).

The polymer dye may be prepared synthetically by polymerization of monomers, which leads to formation of a highly conjugated fluorescent backbone. Capping may be carried out on the polymer by activation using appropriate functionalities, which results in a polymer capable of being conjugated to a binding partner. Alternatively, the polymer may be activated for conjugation or attachment of an acceptor dye by attaching appropriate functionalities off the polymer backbone. The activated polymers may be conjugated, for example, to a binding partner, an acceptor dye or a quenching moiety. Any appropriate binding partner may be employed, for example, an antibody, followed by purification, for example, by using standard procedures. Functional groups can be selected from the group consisting of amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof for conjugation to a substrate or binding partner.

The polymer dye may comprise fluorescent polymer dyes having aryl and/or heteroaryl monomer subunits including, but not limited to, dihydrophenanthrene (DHP), fluorene, and combinations thereof. In some embodiments, the polymer dye can have the structure of Formula I:

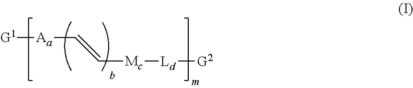

wherein,
   each monomer A is independently an aromatic co-monomer
   or a heteroaromatic co-monomer;
each optional M is an aromatic co-monomer or a heteroaromatic co-monomer; each optional L is a linker;
each G¹ and G² is a modified polymer terminus or an unmodified polymer terminus;
each a is a mol % from 10 to 100%, each c is a mol % from 0 to 90%, and each d is a mol % from 0 to 25%; each b is independently 0 or 1; and each m is an integer from 1 to about 10,000.

As used throughout the disclosure, “a”, “b, “c”, and “d” define the mol % of each unit which each can be evenly or randomly repeated.

Each monomer A may be substituted with a water-solubilizing group and/or an optional functional group that can be conjugated with, for example, an acceptor dye, binding partner or quenching moiety. Each monomer A in polymers haying a structure of Formula I may be the same monomer. Each monomer A in polymer dye having a structure of Formula I may be a different monomer. Monomer A may be, for example, a 9,10-phenanthrenedione-based monomer (e.g., a dihydrophenanthrene (DHP)-based monomer), a fluorene-based monomer, a fluorenooxepine-based monomer.

Monomer A may be a DHP-based, fluorene-based, or carbazole monomer having, for example, a structure of Formula (II):

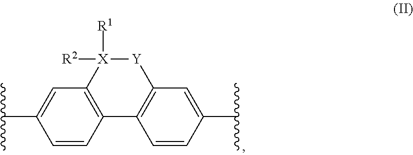

As used herein, each “X” may independently be a C, N or Si.

As used herein, each “Y” is independently selected from the group consisting of CH₂, CR¹R², SiR¹R², or a bond. When Y is a bond, X is directly bonded to both rings.

As used throughout this disclosure, each “R¹” is independently a water-solubilizing moiety, alkene, alkyne, cycloalkyl, haloalkyl, (hetero)aryloxy, (hetero)arylamino, PEG, carboxylic acid, ammonium alkyl salt, ammonium alkyloxy salt, ammonium oligoether salt, sulfonate alkyl salt, sulfonate alkoxy salt, sulfonate oligoether salt, sulfonamido oligoether, sulfonamide, sulfinamide, phosphonamidate, phosphinamide,

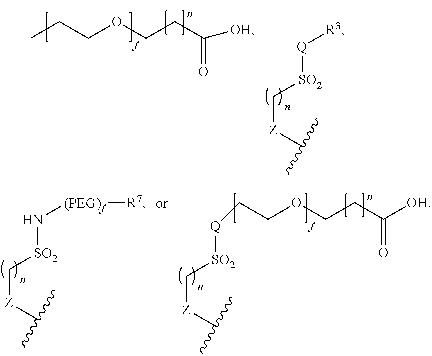

As used herein, each “R²” is independently a water-solubilizing moiety, a linker moiety, H, alkyl, alkene, alkyne, cycloalkyl, haloalkyl, (hetero)aryloxy, (hetero)arylamino, sulfonamide-PEG, phosphoramide-PEG, ammonium alkyl salt, ammonium alkyloxy salt, ammonium oligoether salt, sulfonate alkyl salt, sulfonate alkoxy salt, sulfonate oligoether salt, sulfonamido oligoether, sulfonamide, sulfinamide, phosphonamidate, phosphinamide,

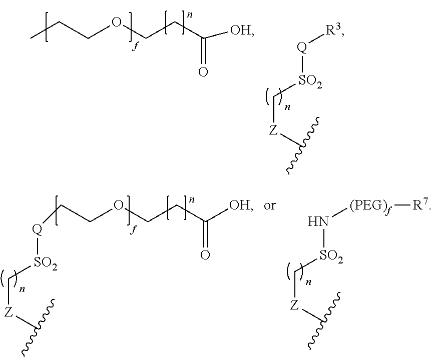

As used herein, each “R³” may independently be a water-solubilizing moiety. Each “R³” may independently be H, alkyl, alkene, alkyne, cycloalkyl, haloalkyl, alkoxy, (hetero)aryloxy, aryl, (hetero)arylamino, or a PEG group.

As used herein, each “R⁴” is independently a H, alkyl, PEG, a water-solubilizing moiety, a linker moiety, a chromophore, carboxylic amine, amine, carbamate, carboxylic acid, carboxylate ester, maleimide, activated ester, N-hydroxysuccinimidyl, hydrazine, hydrazide, hydrazone, azide, alkyne, aldehyde, or thiol, or protected groups thereof.

As used herein, each “R⁷” is H, hydroxyl, C₁-C₁₂alkyl, C₂-C₁₂alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, C₂-C₁₂ carboxylic acid, C₂-C₁₂ carboxylate ester, or C₁-C₁₂ alkoxy.

As used herein, each “Q” is independently a bond, NR⁴ or —CH₂.

As used herein, each “Z” is independently CH₂, O, or NR⁴.

As used herein, in some cases, at least one of R¹, R², R³, or R⁴ comprises a water-solubilizing moiety.

As used herein, each f is independently an integer from 0 to 50, 1-50, 2-40, 5-20; and each n is independently an integer from 1 to 20.

The DHP-based monomer A may, for example, have the structure of Formula (III):

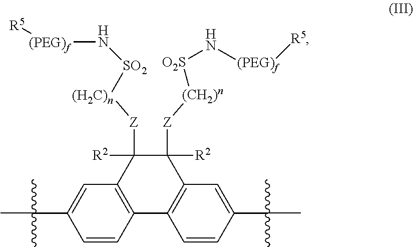

The DHP monomer may, for example, have the structure of Formula (V):

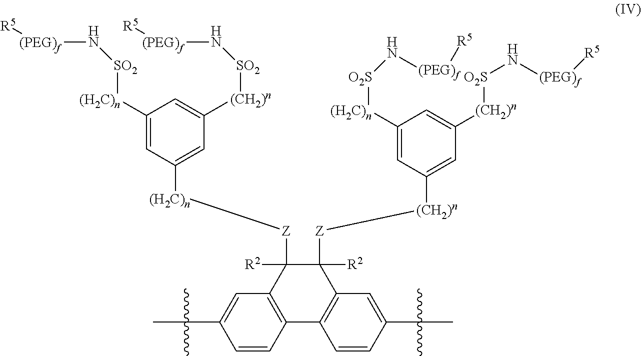

Monomers A in polymers having a structure of Formula I may be fluorene-based monomers, or carbazole-based monomers, for example, having the structure of Formula (Va) or (VP), wherein X is C or N, respectively:

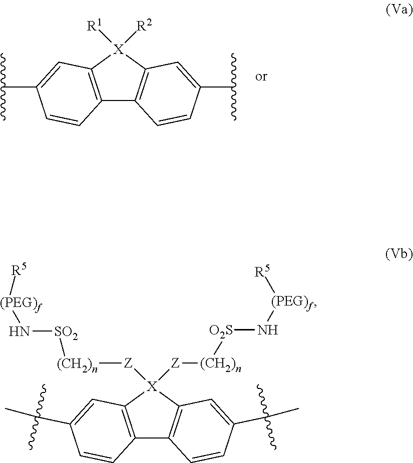

Monomers A may also be bridged monomers. For example, bridged monomers may have the structure of Formula (VIa), (VIb) or (VIc):

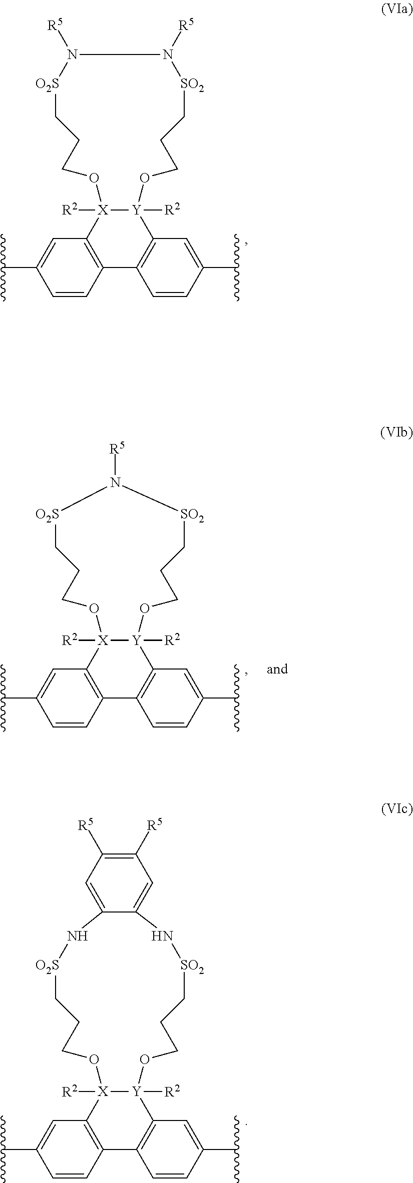

Monomers A in polymers having a structure of Formula I may be oxepine-based monomers (e.g., fluorenooxepine-based monomers), for example, having the structure of Formula (VIIa), (VIIb), or (VIIc):

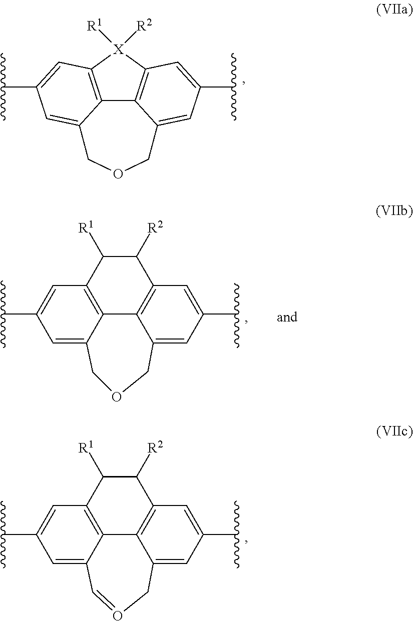

Monomers A in polymers having a structure of Formula I may be a binapthynyl monomer as described in WO 2022/013198, which is incorporated herein by reference in its entirety. The binaphthynyl-based monomers may have the Formula (VIId):

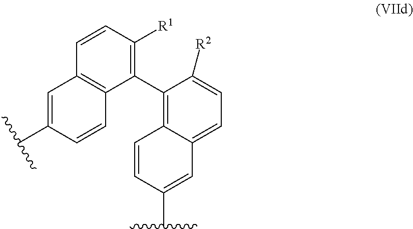

Each optional M in polymers having a structure of Formula I may be a polymer modifying unit evenly or randomly distributed along the polymer chain and may optionally be substituted with one or more optionally substituted R¹, R², R³, or R⁴ groups, as defined herein.

Each optional M may be optionally substituted ethylene or ethynylene. Each M may be an optionally substituted ethylene moiety, i.e., carbon-carbon double bonds having the formula —CR═CR—, wherein each R is independently H, alkyl, alkenyl, alkynyl, cycloalkyl, haloalkyl, alkoxy, (hetero)aryloxy, aryl, (hetero)arylamino, a PEG group, an ammonium alkyl salt, an ammonium alkyloxy salt, an ammonium oligoether salt, a sulfonate alkyl salt, a sulfonate alkoxy salt, a sulfonate oligoether salt, a suifonamido oligoether, or a moiety

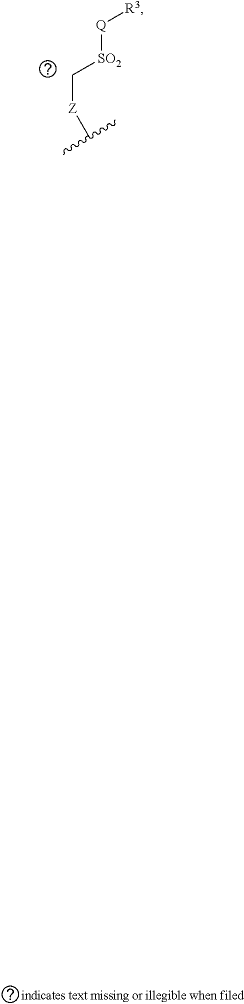

Each M may be an ethynylene moiety, i.e., carbon-carbon triple bonds having the formula —≡C—.

Each optional M may be evenly or randomly distributed along the polymer main chain. Each optional M may be a bandgap-modifying monomer. Each optional M may independently be

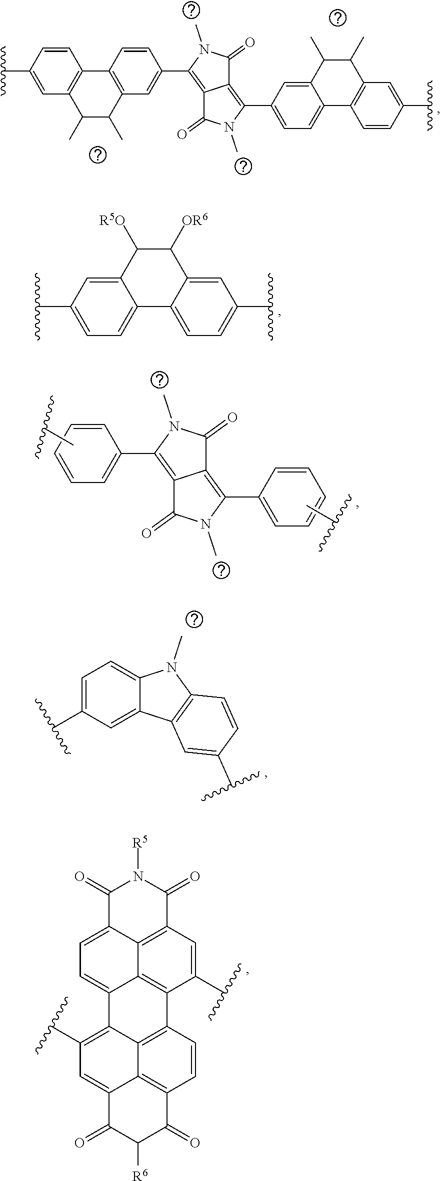

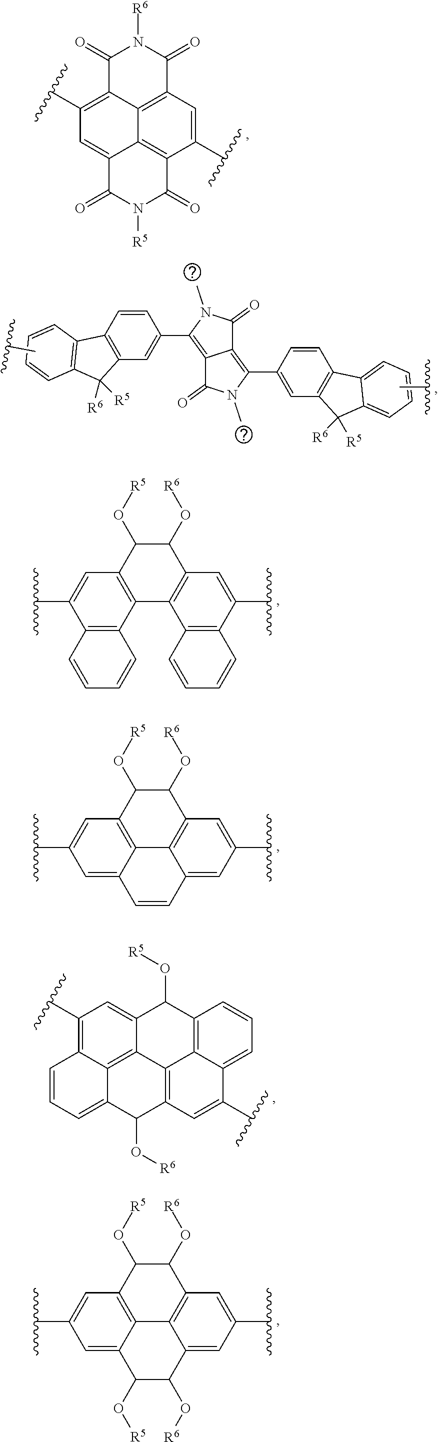

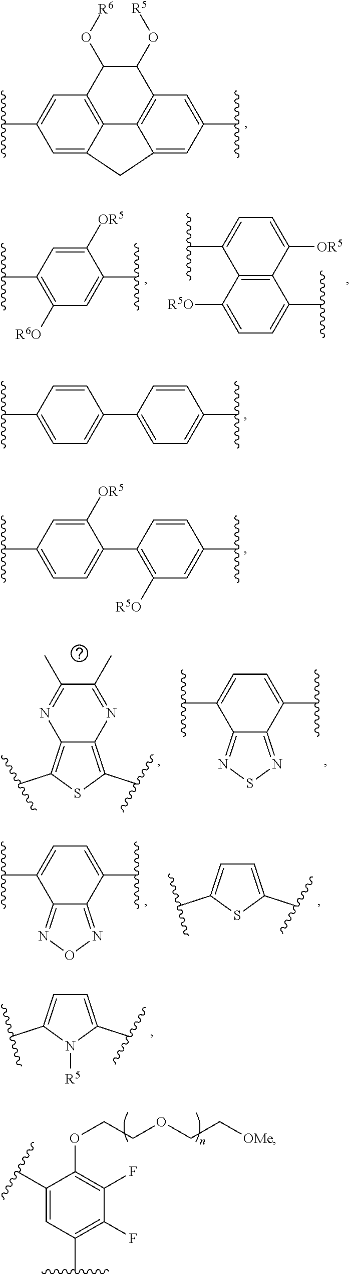

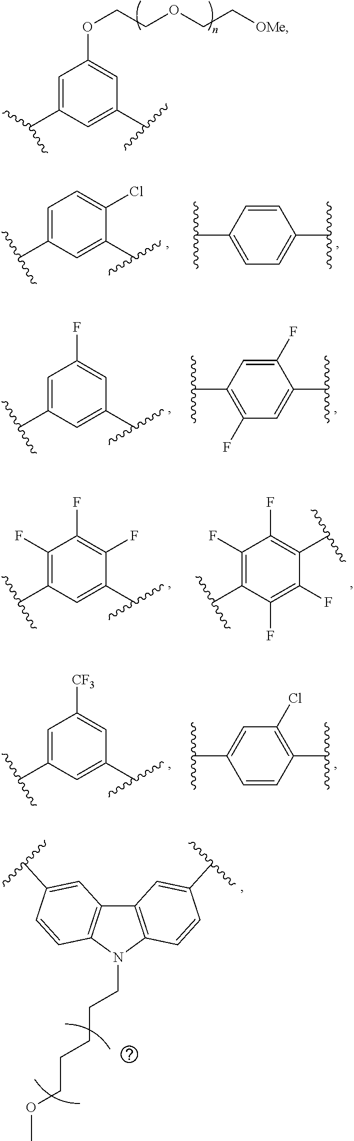

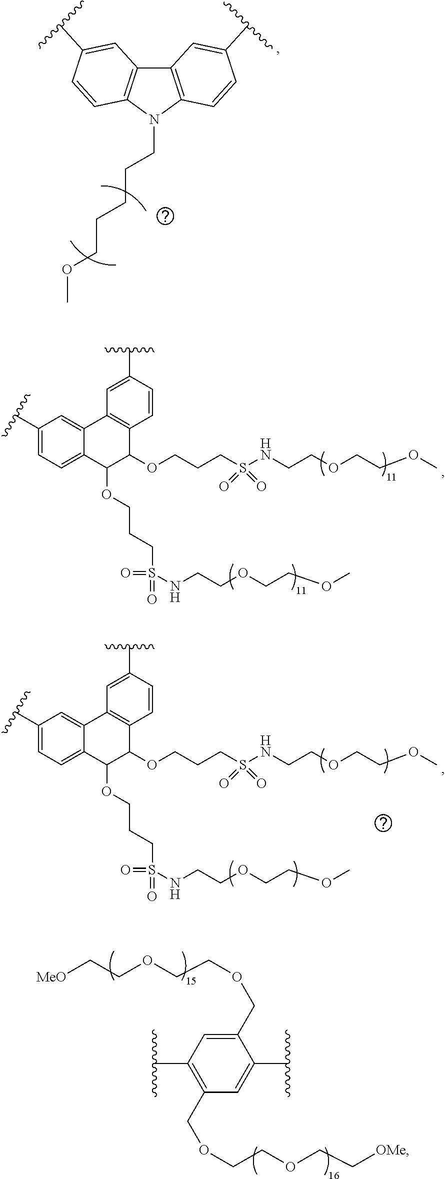

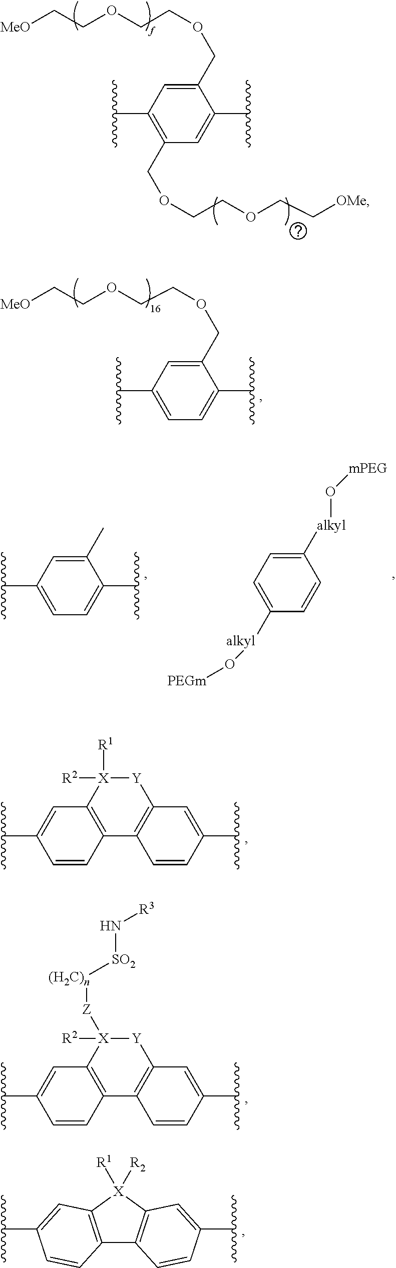

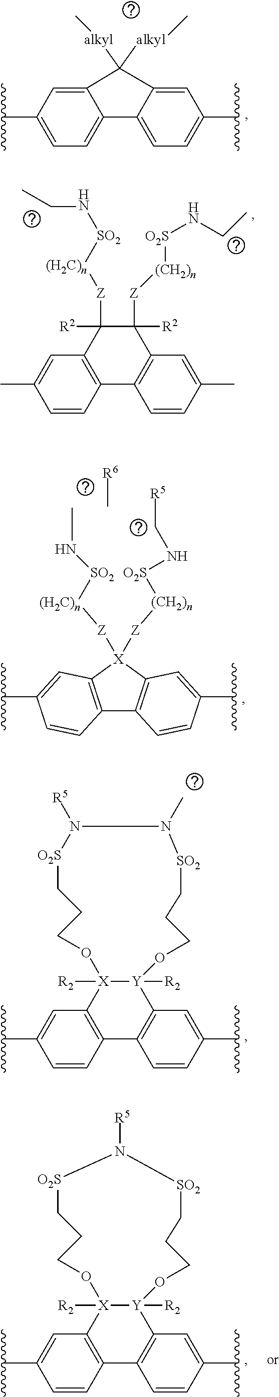

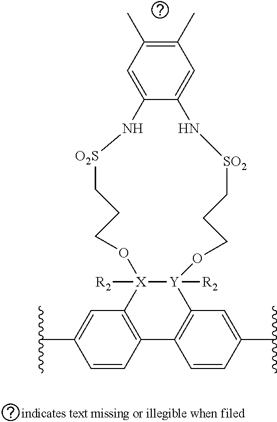

wherein, each M can be substituted, and terminated with a functional group selected from amine, carbamate, carboxylic acid, carboxylate, maleimide, activated ester, N-hydroxysuccinimidyl, hydrazine, hydrazide, hydrazone, azide, alkyne, aldehyde, thiol, amide, sulfonamide, ether, thioether, thiocarbamate, hydroxyl, iodoacetyl, hydrazido, hydrazino, ketone, phosphine, epoxide, urea, thiourea, thioester, imine, disulfides, and protected groups thereof for conjugation to another substrate, acceptor dye, molecule or binding partner.

As used herein, each “R⁵” is independently H, C₁-C₂₀ alkyl, C₂-C₂₀alkenyl, C₂-C₂₀ alkynyl, C₃-C₂₀ cycloalkyl, C₁-C₂₀ haloalkyl, C₁-C₂₀ alkoxy, C₂-C₂₆ aryloxy, C₂-C₂₆ heteroaryloxy, C₂-C₂₆ arylamino, or C₂-C₂₆heteroarylamino.

Each optional L in the polymer having the structure of Formula I is a linker. Each L may be an aryl or heteroaryl group evenly or randomly distributed along the polymer main chain. L may be an aryl or heteroaryl group evenly or randomly distributed along the polymer main and may optionally be substituted with one or more pendant chains terminated with a functional group selected from the group consisting of amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof for conjugation to a substrate or binding partner. Each optional L may independently be

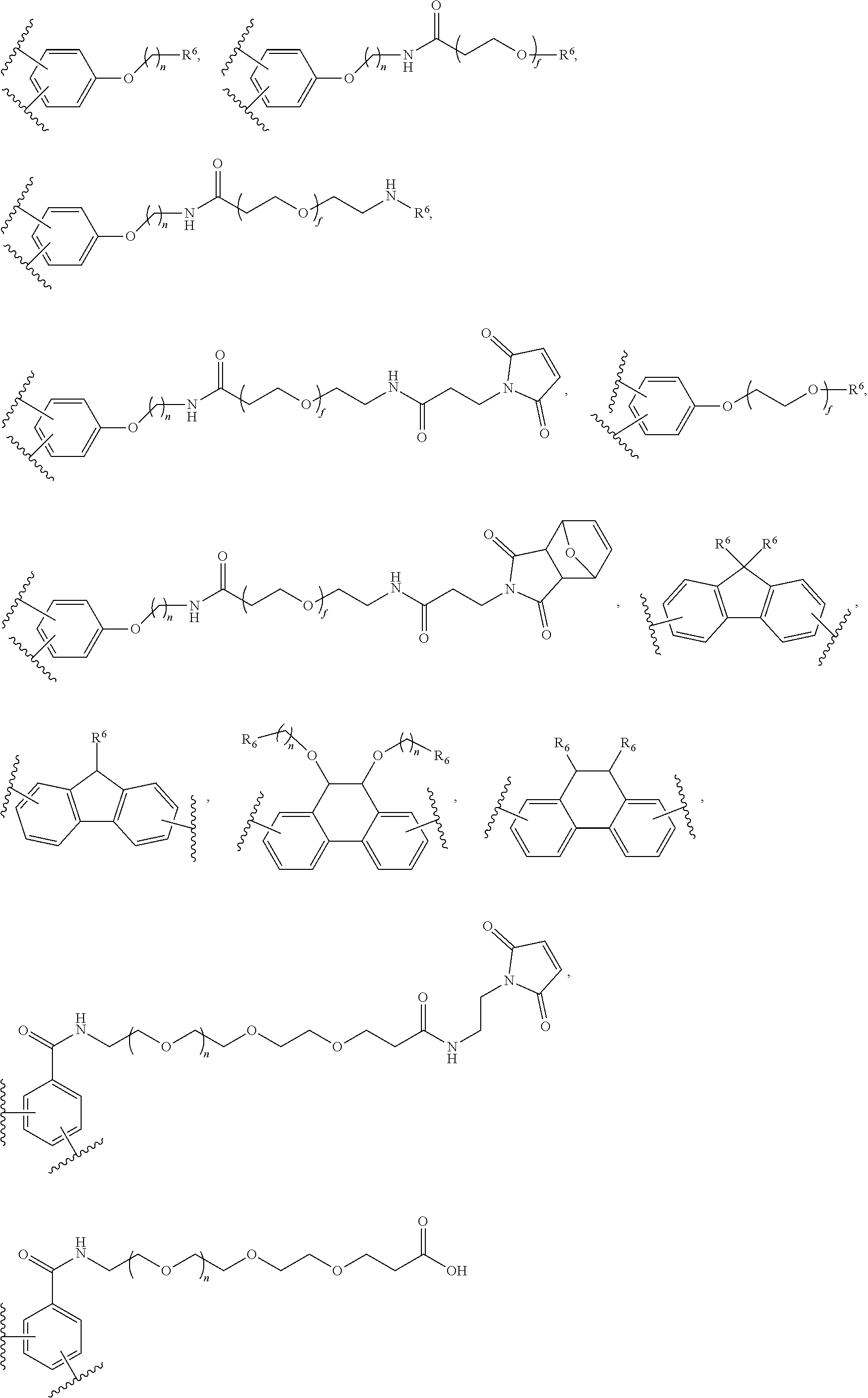

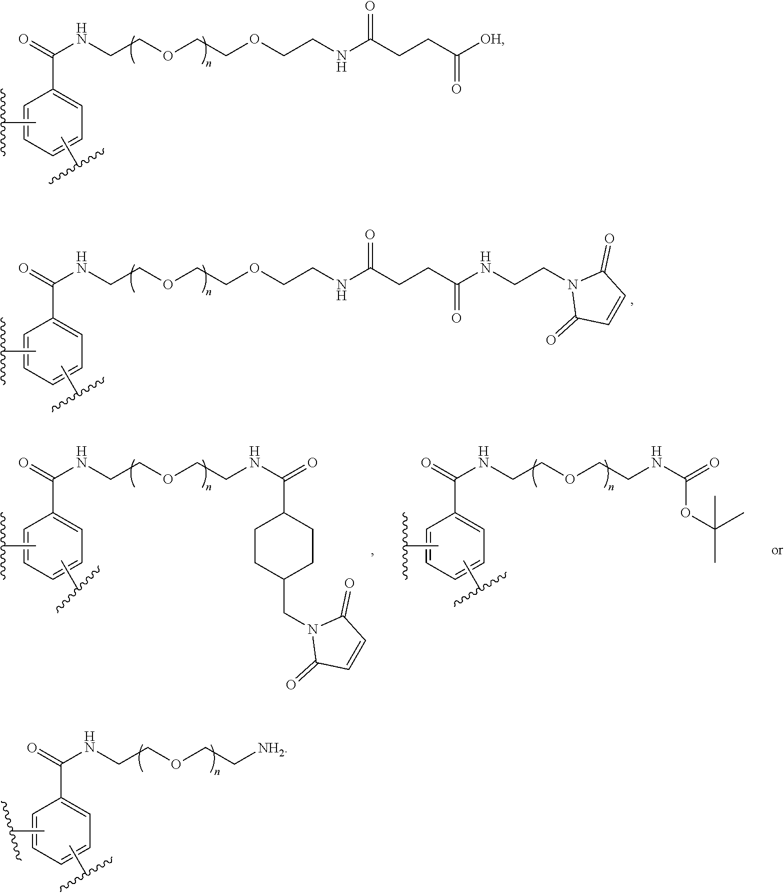

As used herein, each R⁶ is independently H, OH, SH, NHCOO-t-butyl, (CH₂)ₙCOOH, (CH₂)ₙCOOCH₃, (CH₂)ₙNH₂, (CH₂)ₙNH—(CH₂)ₙ—CH₃, (CH₂)ₙNHCOOH, (CH₂)ₙNHCO—(CH₂)ₙ—CO—(CH₂)ₙ—CH₃, (CH₂)ₙNHCOO—(CH₂)ₙ—CH₃, (CH₂)ₙNHCOOC(CH₃)₃, (CH₂)ₙNHCO(C₃-C₁₂)cycloalkyl, (CH₂)ₙNHCO(CH₂CH₂O)_{f}, (CH₂)ₙNHCO(CH₂)ₙCOOH, (CH₂)ₙNHCO(CH₂)ₙCOO(CH₂)ₙCH₃, (CH₂)ₙ(OCH₂CH₂)_{f}OCH₃, N-maleimide, halogen, C₂-C₁₂ alkene, C₂-C₁₂ alkyne, C₃-C₁₂ cycloalkyl, C₁-C₁₂ halo alkyl, C₁-C₁₂ (hetero)aryl, C₁-C₁₂ (hetero)arylamino, benzyl optionally substituted with one or more halogen, hydroxyl, C₁-C₁₂ alkoxy, or (OCH₂CH₂)_{f}OCH₃,

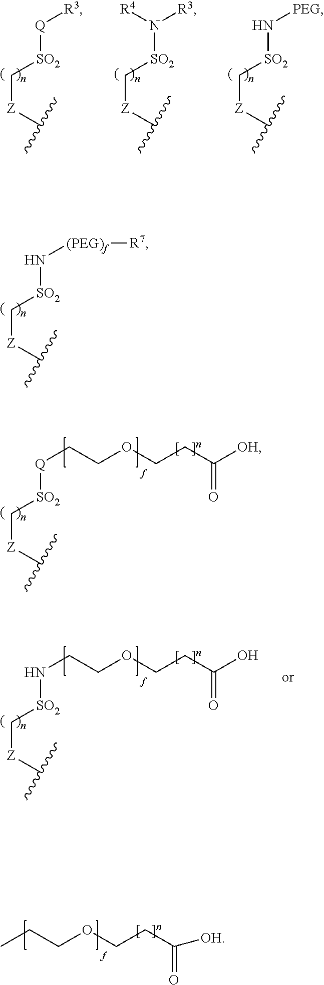

Each G¹ and G² may independently be hydrogen, halogen, alkyne, optionally substituted aryl, optionally substituted heteroaryl, halogen substituted aryl, silyl, diazonium salt, triflate, acetyloxy, azide, sultanate, phosphate, boronic acid substituted aryl, boronic ester substituted aryl, boronic ester, boronic acid, optionally substituted dihydrophenanthrene (DHP), optionally substituted tetrahydropyrene (THP), optionally substituted fluorene, or aryl or heteroaryl substituted with one or more pendant chains terminated with a functional group selected from amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof that may be conjugated to a substrate or binding partner. Each G¹ and G² may independently be

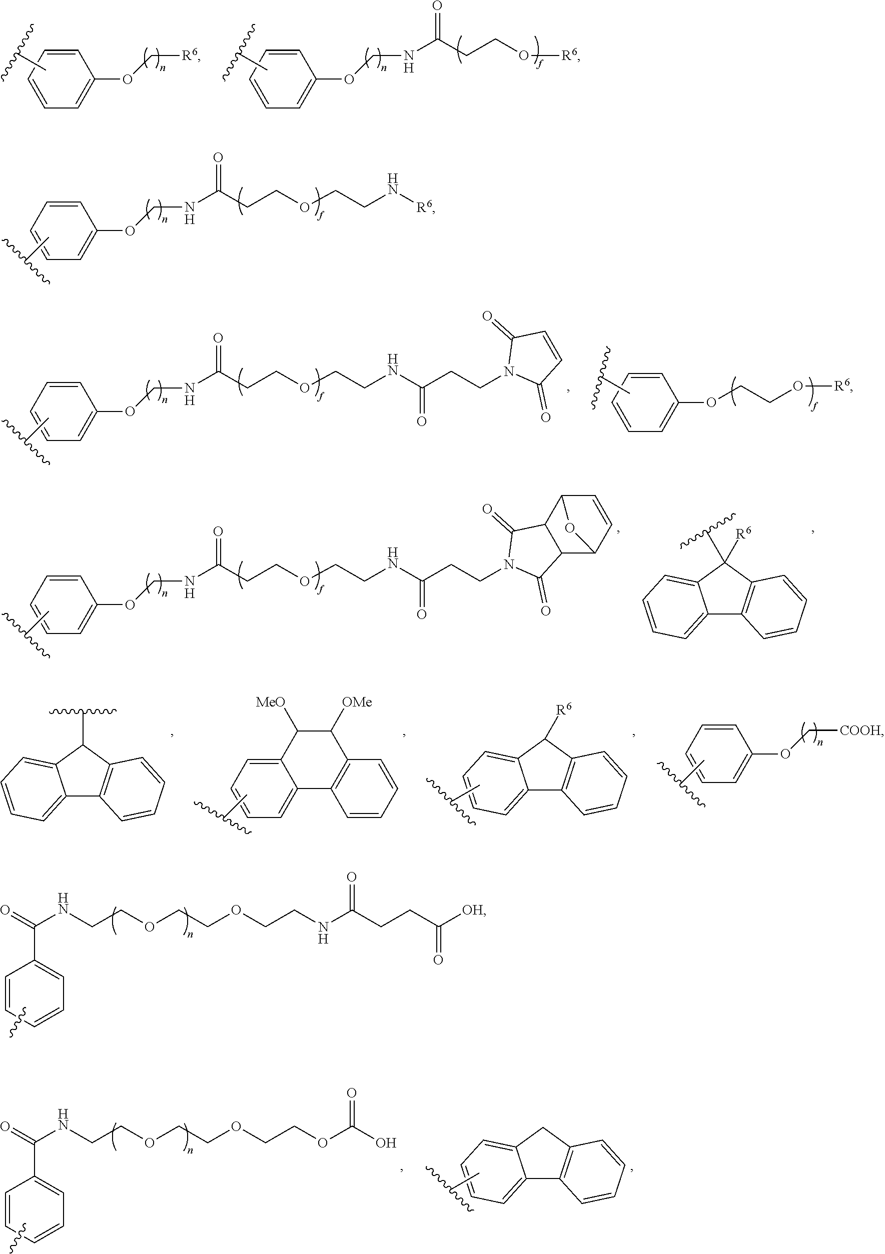

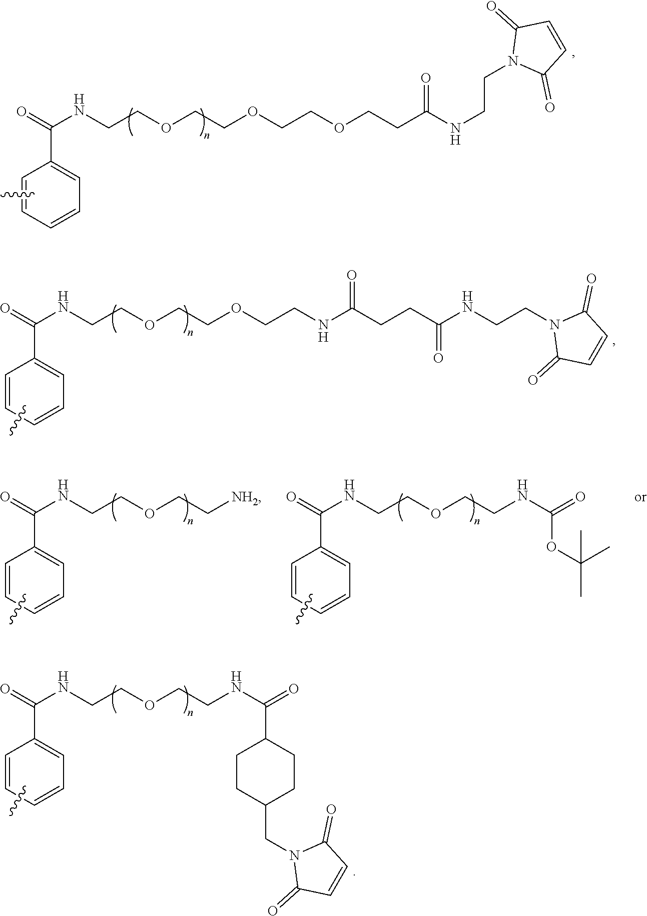

Capping units can be conjugated to a polymer backbone of this invention via mechanisms known in the art and as taught in taught in US Published Application No. 2019/0144601 and U.S. Published Application No. 2020/0190253, both of which are incorporated herein in their entireties.

Polymer dyes having a structure of Formula (I) may utilize dihydrophenanthrene (DHP), fluorene, carbazole and/or binaphthyl monomers, and combinations of DHP, fluorene, carbazole and/or binaphthyl monomers. The polymer dyes having a structure of Formula (I) may utilize a monomer A having a structure according to Formula (II), wherein each “X” may independently be a C, N or Si, and each “Y” may independently be CH₂, CR¹R², SiR¹R², or a bond. When Y is a bond, X is directly bonded to both rings.

For example, the polymer dyes may have the structure of Formula (VIII):

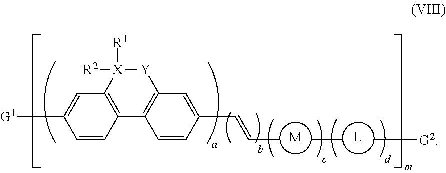

The polymer dyes may have the structure of Formula (IX):

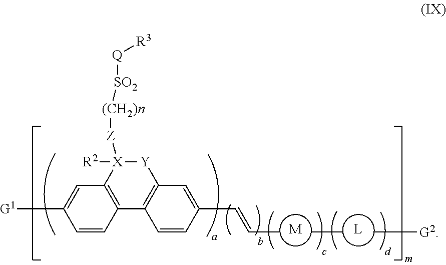

In some cases, the polymer may have the structure of Formula (X):

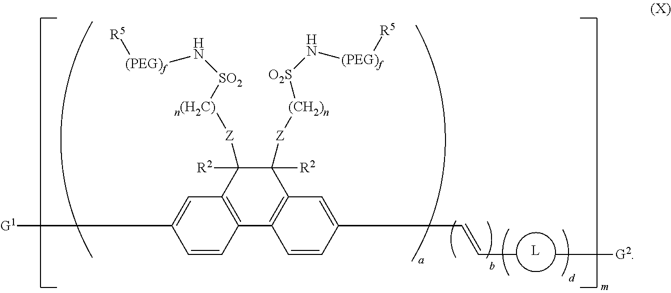

In some cases, the polymer may have the structure of Formula (X):

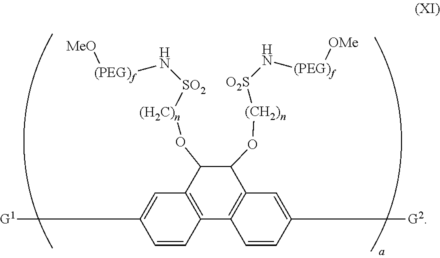

In some cases, the polymer is a copolymer and has the structure of Formula (XII):

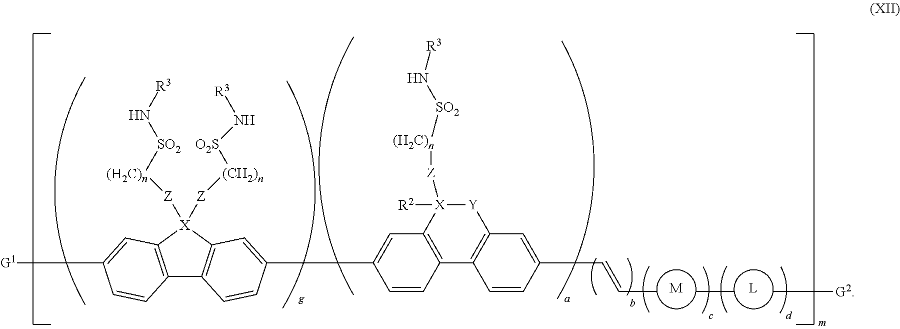

In some cases, the polymer is a copolymer and has the structure of Formula (XIII):

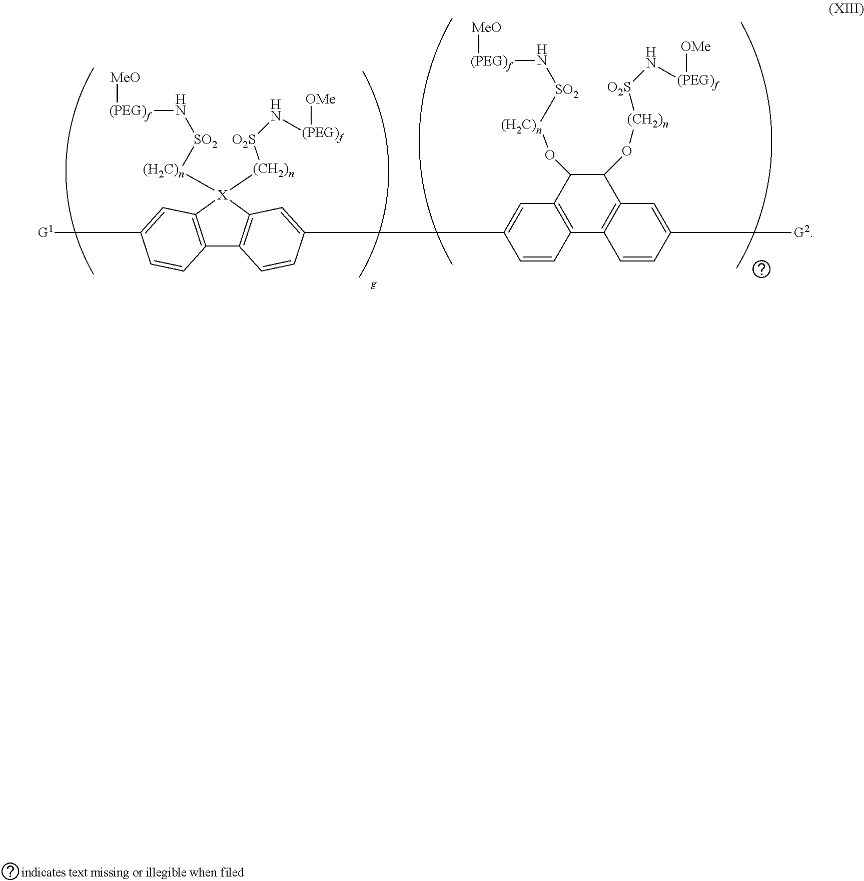

In some cases, the polymer is a copolymer and has the structure of Formula (XIV):

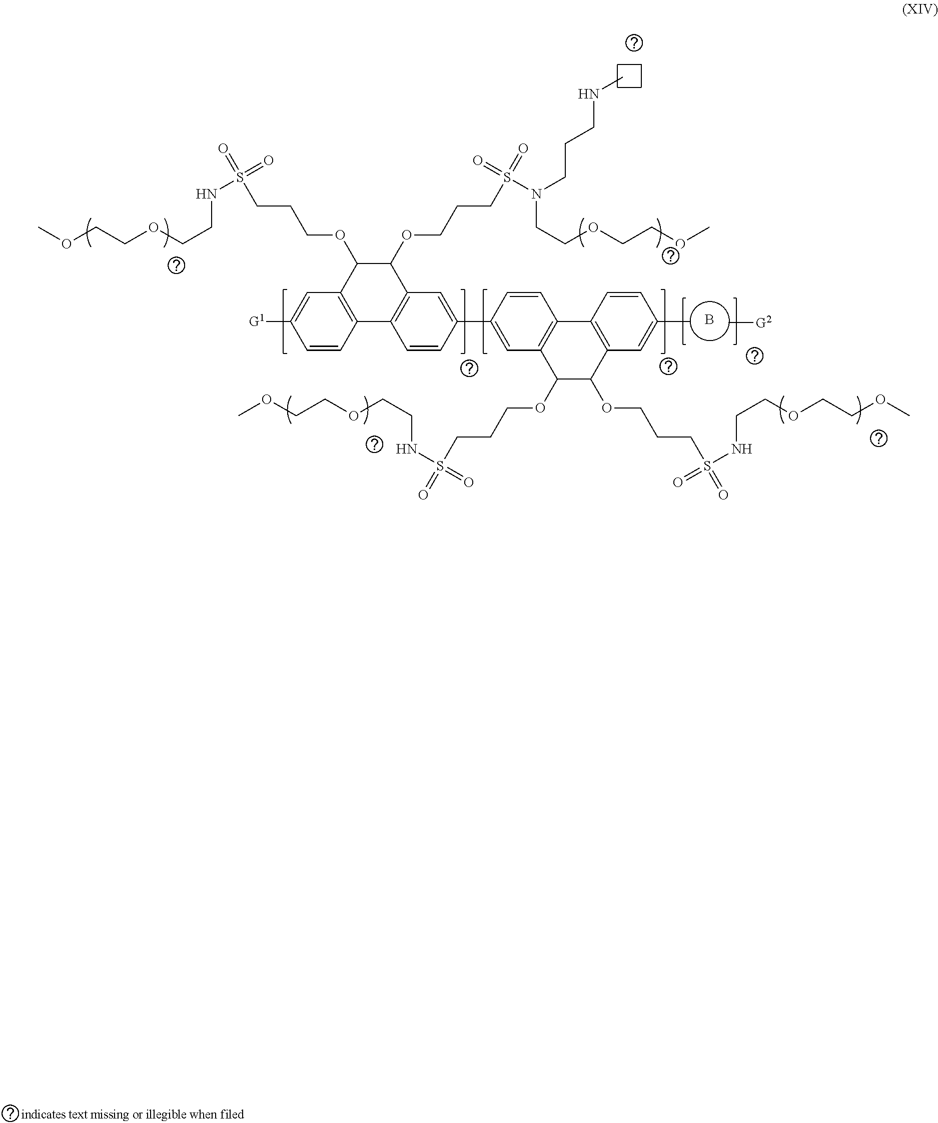

wherein:
each B is independently selected from the group consisting of an aromatic co-monomer, a heteroaromatic co-monomer, a bandgap-modifying monomer, optionally substituted ethylene, and ethynylene;
each E is an independently selected chromophore, functional moiety, or binding partner;
subscripts n and m are independently integers ranging from 1 to 10,000;
subscript p is an integer ranging from 0 to 10,000; and
the sum of subscripts n, m, and p ranges from 2 to 10,000; and
each t is an integer ranging from 1 to 20.

Polymers as described herein can be characterized by a minimum number average molecular weight (Mn) of greater than 5,000 g/mol, greater than 10,000 g/mol, greater than 15,000 g/mol, greater than 20,000 g/mol, greater than 25,000 g/mol, greater than 30,000 g/mol, greater than 40,000 g/mol, greater than 50,000 g/mol, greater than 60,000 g/mol, greater than 70,000 g/mol, greater than 80,000 g/mol, greater than 90,000 g/mol, or greater than 100,000 g/mol.

Polymers as described herein can be characterized by a minimum weight average molecular weight (Mw) of greater than 5,000 g/mol, greater than 10,000 g/mol, greater than 15,000 g/mol, greater than 20,000 g/mol, greater than 25,000 g/mol, greater than 30,000 g/mol, greater than 40,000 g/mol, greater than 50000 g/mol, greater than 60,000 g/mol, greater than 70,000 g/mol, greater than 80,000 g/mol, greater than 90,000 g/mol, or greater than 100,000 g/mol.

The term “Mw” refers to weight average molecular weight, and “Mn” refers to number average molecular weight. Number average and weight average molecular weight values can be determined by gel permeation chromatography (GPC) using polymeric standards (e.g., polystyrene or like material).

### Conjugated Polymers

The polymers having a structure of Formula (I) can be conjugated with an acceptor dye, functional groups, including quenching moieties, and/or binding partners. The conjugation may occur at various locations on the polymer, such as monomer A, L, G1 or G2 in the polymer structure of Formula (I).

For example, as described in US Published Application No. 2019/0144601, which is incorporated herein by reference in its entirety, acceptor dyes may be attached to the polymer through a linker L:

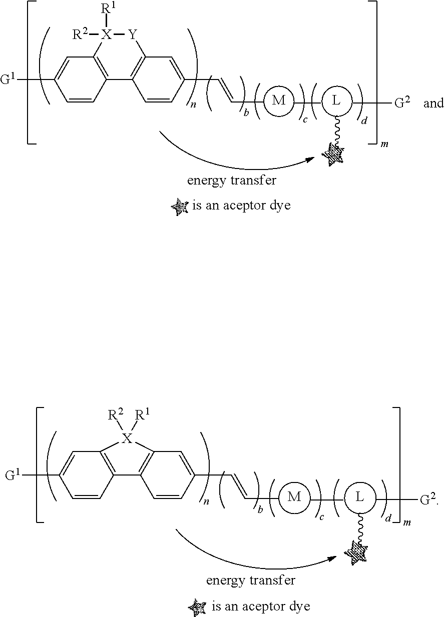

Tandem polymer dyes and other functionalized polymers can be prepared by modification of polymer intermediates after polymerization, as described in US Published Application No. 2020/0190253, which is incorporated herein by reference in its entirety. For example, pendant solubilizing groups having the Formula (XVI) may be attached to Monomer A:

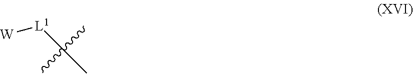

and converted to functionalized solubilizing groups according to Formula (XVII):

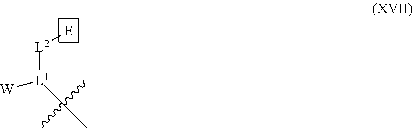

wherein W is a water solubilizing moiety and L¹ and L² are linking moieties. In some instances, the L1-W group having a structure of Formula (XVI) may be R¹ or R². In some embodiments, each E is an independently selected chromophore, functional moiety, such as a quenching moiety, or binding partner. In some embodiments, each E is an independently selected chromophore (e.g., and independently selected fluorophore). In some embodiments, all of the E moieties in the polymer have the same structure. In some embodiments, E moieties in the polymer having different structures. Water solubilizing moieties W in groups according to Formula (XVI) and Formula (XVII) may be, for example, an ammonium alkyl salt, an ammonium alkyloxy salt, an ammonium oligoether salt, a sultanate alkyl salt, a sulfonate alkoxy salt, a sulfonate oligoether salt, a suifonamido oligoether, an oligo(ethylene glycol), or a poly(ethylene glycol).

Linking moieties L¹ and L² may independently be, but are not limited to, a covalent bond, C₁₋₆ alkylene, 2- to 8-membered heteroalkylene. In some embodiments, the linker is a single atom, a linear chain, a branched chain, a cyclic moiety. In some embodiments, the linker is chain of between 2 and 100 backbone atoms (e.g., carbon atoms) in length, such as between 2 and 50 backbone atoms in length or between 2 and 20 atoms backbone atoms in length. In certain cases, one, two, three, four or five or more carbon atoms of a linker backbone can be optionally replaced with sulfur, nitrogen, or oxygen. The bonds between backbone atoms can be saturated or unsaturated; typically, not more than one, two, or three unsaturated bonds will be present in a linker backbone. The linker can include one or more substituent groups (e.g., an alkyl group or an aryl group). A linker can include, without limitation, oligo(ethylene glycol); ethers; thioethers; tertiary amines; and alkylene groups (i.e., divalent alkyl radicals), which can be straight or branched. The linker backbone can include a cyclic group, for example, a divalent aryl radical, a divalent heterocyclic radical, or a divalent cycloalkyl radical, where 2 or more atoms, e.g., 2, 3, or 4 atoms, of the cyclic group are included in the backbone.

L¹ can comprise a sulfonamide, a sulfinamide, a disulfonamide, a disulfinamide, a sultam, an amide, a secondary amine, a phosphonamide, a phosphinamide, a phosphonamidate, a selenonamide, or a seleninamide. In some embodiments. L¹ comprises a sulfonamide, an amide, secondary amine, or a phosphonamide. In some such embodiments, L² comprises a linear or branched, saturated or unsaturated C₁₋₃₀ alkylene group; wherein one or more carbon atoms in the C₁₋₃₀ alkylene group is optionally and independently replaced by O, S, NR^{a}; wherein two or more groupings of adjacent carbon atoms in the C₁₋₃₀ alkylene are optionally and independently replaced by —NR^{a}(CO)— or —(CO)NR^{a}—; and wherein each Ra is independently selected from H and C₁₋₆ alkyl.

Polymers can be functionalized by covalently bonding an internal position of L¹ in a pendant solubilizing group according to Formula (XVI) to a first end of linker moiety L² in a first step, and then covalently bonding a dye, or other functional group or binding partner E, to a second end of linker moiety L² in a second step. In some embodiments, a nitrogen atom in L¹ (e.g., an amide nitrogen, a sulfonamide nitrogen, or a phosphonamide nitrogen) is alkylated using a linker moiety L² having a suitable leaving group at the first end of the linker moiety. In some embodiments, for example, the leaving group is a halogen (e.g., chloro, bromo, or iodo). In some embodiments the leaving group is a sulfonate (i.e., —OS(O)₂R, wherein R is alkyl, haloalkyl, aryl, or substituted aryl). Suitable sulfonates include, but are not limited to, mesylate (methanesulfonate), triflate (trifluoromethanesulfonate), besylate (benzene-sulfonate), tosylate (p-toluenesulfonate), and brosylate (4-bromobenzenesulfonate).

Any suitable solvent may be used for alkylation steps during polymer functionalization with group E. Suitable solvents include, but are not limited to, toluene, methylene chloride, ethyl acetate, acetonitrile, tetrahydrofuran, benzene, chloroform, diethyl ether, dimethyl formamide, dimethyl sulfoxide, petroleum ether, and mixtures thereof. Alkylation reactions are typically conducted at temperatures ranging from around 25° C., to about 100° C. for a period of time sufficient install a linking moiety L², or a linked functional group -L²-E, at one or more pendant groups in the polymer. The reaction can be conducted for a period of time ranging from a few minutes to several hours or longer, depending on the polymer and reagents used in the reaction. For example, the reaction can be conducted for around 10 minutes, or around 30 minutes, or around 1 hour, or around 2 hours, or around 4 hours, or around 8 hours, or around 12 hours at around 40° C., or around 50° C., or around 60° C., or around 70° C., or around 80° C.

The second end of the linking moiety L² may comprise a functional group (e.g., an amine or a carboxylic acid) which is used in protected form during the first step (e.g., an alkylation step) and which is then deprotected prior to covalently bonding the dye, or other functional group or binding partner E, to the second end of linking moiety. Examples of amine protecting groups include, but are not limited to, benzyloxycarbonyl; 9-fluorenylmethyloxycarbonyl (Fmoc); tert-butyloxycarbonyl (Sac); allyloxycarbonyl (Alloc); p-toluene sulfonyl (Tos); 2,2,5,7,8-pentamethylchroman-6-sulfonyl (Pmc); 2,2,4,6,7-pentamethyl-2,3-dihydrobenzofuran-5-sulfonyl (Pbf); mesityl-2-sulfonyl (Mts); 4-methoxy-2,3,6-trimethylphenylsulfonyl (Mt); acetamido; phthalimido; and the like. These and other protecting groups for amines, carboxylic acids, alcohols, and further functional groups can be added to and removed from polymers of the present disclosure using known techniques as described, for example, by Green and Wuts (Protective Groups in Organic Synthesis, 4^{th} Ed 2007, Wiley-Interscience, New York).

Addition of dyes, binding partners, and functional groups, such as, for example, quenching moieties, can be conducted using any suitable method. For example, an amide linkage is formed between a deprotected primary amine group of L² and carboxylate-functionalized dye. The dye may be used in activated form, e.g., as a reagent E-C(O)X′ can be used, wherein X′ is a leaving group. Activated carboxylate-functionalized reagents include, but are not limited to, anhydrides (including symmetric, mixed, or cyclic anhydrides), activated esters (e.g., p-nitrophenyl esters, pentafluorophenyl esters, N-succinimidyl esters, and the like), acylazoles (e.g., acylimidazoles, prepared using carbonyl diimidazole, and the like), acyl azides, and acid halides (e.g., acid chlorides). Alternatively, a coupling agent may be used to form a bond the amide linkage between a deprotected primary amine group of L² and carboxylate-functionalized chromophore E-C(O)OH. The coupling agent may be used to form activated dye reagents prior to reaction with polymer amine groups. Any suitable coupling agent may be used. In some embodiments, the coupling agent is a carbodiimide, a guanidinium salt, a phosphonium salt, or a uronium salt. Examples of carbodiimides include, but are not limited to, N,N′-dicyclohexylcarbodiimide (DCC), ethyl-3-(3-dimethylaminopropyl)-carbodiimide (EDC), and the like. Examples of phosphonium salts include, but are not limited to, such as (benzotriazol-1-yloxy)tripyrrolidinophosphonium hexafiuorophosphate (PyBOP); bromotris(dimethylamino)phosphonium hexafluorophosphate (BroP); and the like. Examples of guanidinium/uronium salts include, but are not limited to, N,N,N′-tetramethyl-O—(N-succinimidypuronium tetrafluoroborate (TSTU); O-(benzotriazol-1-yl)-N,N,N′,N′-tetramethyl-uronium hexafluorophosphate (HBTU); 2-(7-aza-1H-benzotriazole-1-yl)-1,1,3,3-tetramethyl-uronium hexafluorophosphate (HATU); 1-[(1-(cyano-2-ethoxy-2-oxoethylidene-aminooxy) dimethylaminomorpholino)]uronium hexafluorophosphate, (COMU); and the like. Solvents, reaction times, and other reaction conditions can be varied as described above depending on factors such as the nature of the particular polymer and dye/functional group.

For example, addition of dyes, binding partners, functional groups, and quenching moieties, may include converting a polymer according to Formula (XVIIIa):

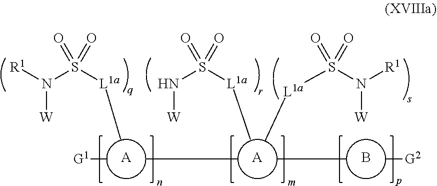

into a polymer according to Formula (XVIII):

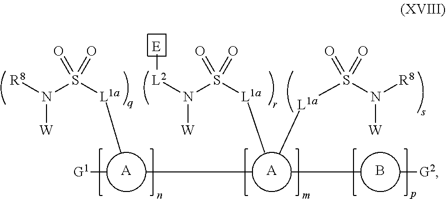

wherein:
each A is independently an aromatic co-monomer or a heteroaromatic co-monomer;
each L1^{a} is independently selected from the group consisting of a covalent bond, Ci₁₋₈ alkylene, C₁₋₈ alkoxy, 2- to 8-membered heteroalkylene, —NHC(O)L^{a}-, —C(O)NHL^{a}-, and —C(O)L^{a}-;
L² is selected from the group consisting of a covalent bond, C₁₋₈ alkylene, 2- to 8-membered heteroalkylene, -L^{b}NHC(O)—, -L^{b}C(O)NH—, -L^{b}C(O)—, —C(O)NHL^{b}-, and C(O)L^{b}-;
L^{a} and L^{b} are independently selected from the group consisting of C₁₋₈ alkylene and 2- to 8-membered heteroalkylene;
W is a water-solubilizing moiety;
each E is an independently selected chromophore, functional moiety, quenching moiety, or binding partner;
each B is independently selected from the group consisting of an aromatic co-monomer, a heteroaromatic co-monomer, a bandgap-modifying monomer, optionally substituted ethylene, and ethynylene;
G¹ and G² are independently selected from an unmodified polymer terminus and a modified polymer terminus;
R⁸ is selected from the group consisting of R¹ as defined herein above, H, or an amine protecting group;
subscripts n and m are independently integers ranging from 1 to 10,000,
subscript p is an integer ranging from 0 to 10,000, and
the sum of subscripts n, m, and p ranges from 2 to 10,000;
subscript q is 1, 2, 3, or 4;
subscript r is 1, 2, 3, or 4;
subscript s is 0, 1, 2, or 3;
subscript t is 1 or 2; and
A and B are distributed randomly or non-randomly in the fluorescent polymer.

Converting the polymer of Formula (XVIIIa) to the polymer according to Formula (XVIII) may include one or more alkylation steps, or one or more amide formation steps, as described above.

In some embodiments, the polymer dye conjugate is a tandem dye conjugate according to Formula (XIX):

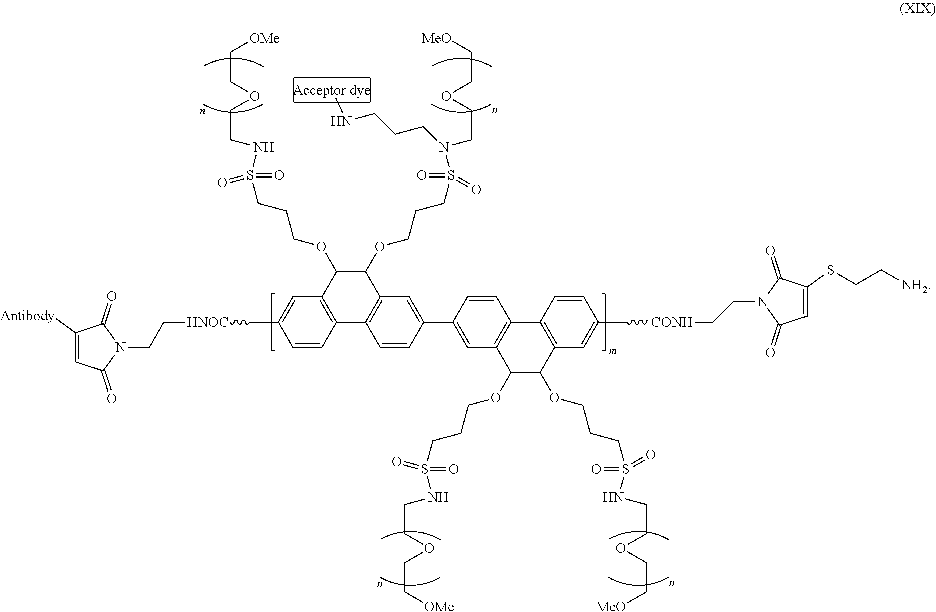

Any suitable chromophore or fluorophore can be used for polymer functionalization. In general, suitable chromophores and fluorophores have a reactive group (e.g., a carboxylate moiety, an amino moiety, a haloalkyl moiety, or the like) that can be covalently bonded to the pendant solubilizing groups (e.g., via linking moieties L² as described above). Examples of suitable chromophores and fluorophores include, but are not limited to, those described in U.S. Pat. Nos. 7,687,282; 7,671,214; 7,446,202; 6,972,326; 6,716,979; 6,579,718; 6,562,632; 6,399,392; 6,316,267; 6,162,931; 6,130,101; 6,005,113; 6,004,536; 5,863,753; 5,846,737; 5,798,276; 5,723,218; 5,696,157; 5,658,751; 5,656,449; 5,582,977; 5,576,424; 5,573,909; and 5,187,288, which patents are incorporated herein by reference in their entirety. Polymer dyes and monomers are also described in US2020/0190253, which is incorporated herein by reference in its entirety.

For example, E can be FITC, CY3B, Cy55, Alexa 488, Texas red, Cy5, Cy7, Alexa 750, or 800CW.

For example, the chromophore E can be a boron-dipyrromethene moiety having the structure:

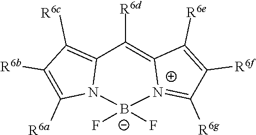

wherein
six of R^{6a}, R^{6b}, R^{6c}, R^{6d}, R^{6e}, R^{6f}, and R^{6g} are independently selected from H, halogen, C₁₋₆ alkyl, C₆₋₈ cycloalkyl, C₆₋₁₀ aryl, C₇₋₁₆ arylalkyl, C₁₋₆ acyl, and —SO₃H; and wherein one of R^{6a}, R^{6b}, R^{6c}, R^{6d}, R^{6e}, R^{6f}, and R^{6g} is the linking moiety -L²-. R^{6a} and R^{6c} can independently be selected C₁₋₆ alkyl (e.g., methyl or ethyl), and one of R^{6e}, R^{6f}, and R^{6g} is the linking moiety -L²-. In some embodiments, R^{6a} and R^{6c} are methyl and R^{6g} is the linking moiety -L².

The chromophore E can be a cyanine moiety haying the structure:

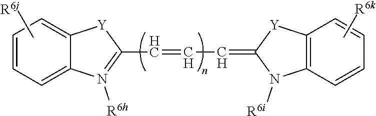

wherein:
R^{6h} and R⁶ⁱ are independently selected from H, C₁₋₆ alkyl, (CH₂)ₜCOOH, (CH₂)ₜSO₃H, and linking moiety L²;
each subscript tis independently an integer from 1 to 10;
R⁶ⁱ and R^{6k} are independently selected from H, halogen, C₁₋₆ alkyl, optionally substituted fused C₆₋₁₀ aryl (e.g., optionally substituted benzo), —SO₃H, —PO₃H₂, —OPO₃H₂, —COOH, and linking moiety L²;
each Y is independently selected from O, S, C(R^{6l})₂, —CH═CH—, and NR^{6l}, where each R^{6l} is independently H or C₁₋₆ alkyl; and
subscript n is an integer from 1 to 6, provided that one and only one of R^{6h}, R⁶ⁱ, R^{6j}, and R^{6k} is the linking moiety -L²-.

The chromophore E can be a coumarin moiety having the structure:

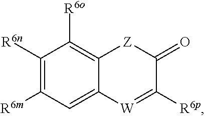

wherein:
W is N or CR^{6p};
Z is O, S, or NR^{6q}; and
each of R^{6m}, R⁶ⁿ, R^{6o}, R^{6p} is independently selected from H, halogen, C₁₋₆ alkyl, —CN, —CF₃, —COOR^{3v}, —CON(R^{3v})₂, —OR^{3v}, and linking moiety -L²-;
R⁶ⁿ is selected from —OR^{3v} and —N(R^{3v})₂
each R⁶ⁿ is independently selected from H, C₁₋₆ alkyl, and linking moiety -L²-; provided that one and only one of R^{6m}, R⁶ⁿ, R^{6o}, R^{6p} and R^{6q} is the linking moiety -L²-.

The chromophore E can be a xanthene moiety having the structure:

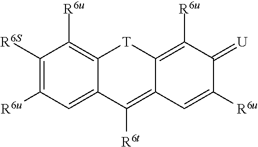

wherein:
T is selected from O, S, C(R^{6u})₂, and NR^{6u}:U is O or N(R^{6u})₂;
each R^{6f} independently selected from H, halogen, C₁₋₆ alkyl, —SO₃H, and linking moiety -L²-
R^{6s} is selected from H, —N(R^{6u})₂, and linking moiety -L²-;
R^{6t} is selected from H, C₁₋₆ alkyl, R⁶ v, and linking moiety -L²-;
each R^{6u} is independently H or C₁₋₆ alkyl; and
R^{6v} is selected from:

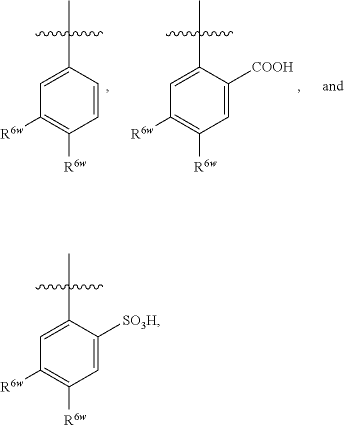

wherein:
each R^{6w} is independently selected from H and linking moiety -L²-; provided that one and only one of R^{6r}, R^{6s}, R^{6t}, and Re R^{6v} is linking moiety -L²-.

The xanthene moiety can be a fluorescein, wherein T and U are O; R^{6s} is OH, and R^{6t} is:

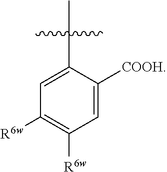

The xanthene moiety can be an eosin, wherein T and U are O; R^{6s} is OH, each R^{6r} is halogen (e.g., bromo), and R^{6t} is:

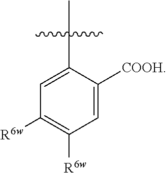

The xanthene moiety can be a rhodamine, wherein T is O; U is N(R^{6u})₂ (e.g., ═NH₂⁺); R is —N(R^{6u})₂ (e.g., —NH₂), and R^{6t} is:

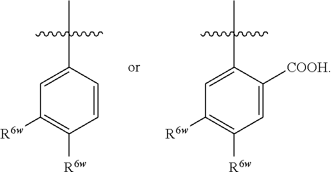

The xanthene moiety can be a shod pine having the structure:

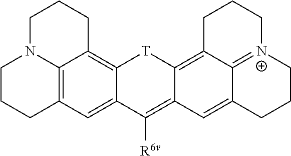

wherein R^{6v} is selected from:

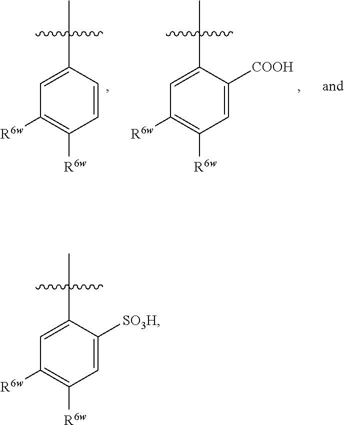

one R^{6w} is H, and the other R^{6w} is linking moiety -L²-.

Other functional moieties, in addition to chromophores, can be appended to functionalized polymers using the methods provided herein. For example, a functional moiety “E” can be a biotin, a digoxigenin, a peptide tag such as a FLAG peptide, an oligonucleotide, or a polynucleotide. As used herein, the term “FLAG peptide” refers to an oligopeptide or a polypeptide containing the amino acid sequence Asp-Tyr-Lys-Asp-Asp-Asp-Asp-Asp-Lys (i. e., DYKDDDDK). FLAG peptides and variants thereof are described for example, in U.S. Pat. No. 4,703,004 to Hopp, et al., which patent is incorporated herein by reference. Other peptides that can be used in place of a FLAG peptide include, but are not limited to, HA peptide tags containing the sequence Tyr-Pro-Tyr-Asp-Val-Pro-Asp-Tyr-Ala (i.e., YPYDVPDYA), His₆ peptide tags containing the sequence His-His-His-His-His-His (i.e., HHHHHH), and Myc peptide tags containing the sequence Giu-Gin-Lys-Leu-lie-Ser-Glu-Glu-Asp-Leu (i.e., EQKLISEEDL). The peptide tags can be recognized by antibodies or other binding moieties for use with colorimetric reagents, chemiluminescent reagents, or the like for convenient identification and/or quantification. Nucleotides (e.g., RNA, single-stranded DNA, or double-stranded DNA) can be recognized by a complementary primer or other complementary nucleotide as described, for example, in WO 2016/019929 (Navratil, et al.), which publication is incorporated herein by reference. As used herein, the term “digoxigenin” refers to 3-[(3S,5R,8R,9S,10S,12R,13S,14S,17R)-3,12,14-trihydroxy-10,13-dimethyl-1,2,3,4,5,6,7,8,9,11,12,15,16,17-tetradecahydrocyclopenta[a]-phenanthren-17-yl]-2H-furan-5-one (CAS Registry No. 1672-46-4) and substituted analogs thereof. As used herein, the term “biotin” refers to 5-[(3aS,4S,6aR)-2-oxohexahydro-1H-thieno[3,4-d]imidazol-4-yl]pentanoic acid (CAS Registry No. 58-85-5) and substituted analogs thereof.

The polymer dyes may be conjugated to different specificities of binding partners, e.g., target-analyte specific antibodies, in order to synthesize a binding partner-dye conjugate such as CD19-SN v428, CD20-SN v605, etc.

As used herein, “binding partner” refers to any molecule or complex of molecules capable of specifically binding to a target analyte. The binding partner may be, for example, a protein (e.g., an antibody or an antigen-binding antibody fragment), a small organic molecule, a carbohydrate (e.g., a polysaccharide), an oligonucleotide, a polynucleotide, a lipid, an affinity ligand, an aptamer, or the like. In some embodiments, the binding partner is an antibody or fragment thereof. In some embodiments, the binding partner is an antibody or antigen-binding fragment thereof that specifically binds a target analyte. Specific binding in the context of the present invention refers to a binding reaction which is determinative of the presence of a target analyte in the presence of a heterogeneous population. Thus, under certain assay conditions, the specified binding partners bind preferentially to a particular protein or isoform of the particular protein and do not bind in a significant amount to other proteins or other isoforms present in the sample.

In some cases, the antibody includes intravenous immunoglobulin (IVIG) and/or antibodies from (e.g., enriched from, purified from, e.g., affinity purified from) IVIG. IVIG is a blood product that contains IgG (immunoglobulin G) pooled from the plasma (e.g., in some cases without any other proteins) from many (e.g., sometimes over 1,000 to 60,000) normal and healthy blood donors. IVIG is commercially available. Aspects of IVIG are described, for example, in US. Pat. Appl. Pub. Nos. 2010/0150942; 2004/0101909; 2013/0177574; 2013/0108619; and 2013/0011388, which are incorporated herein by reference.

When the binding partners are antibodies, they may be monoclonal or polyclonal antibodies. The term “antibody” as used herein refers to immunoglobulin molecules and immunologically active portions of immunogiobulin (Ig) molecules, for example, which specifically bind to an antigen in a target analyte. Such antibodies include, but are not limited to, polyclonal, monoclonal, mono-specific polyclonal antibodies, antibody mimics, chimeric, single chain, Fab, Fab′ and F(ab′)₂, fragments, Fv, and an Fab expression library. In some cases, the antibody is a monoclonal antibody of a defined sub-class (e.g., IgG1, IgG2, IgG3, or IgG4, IgA, IgD, IgE, IgG2a, IgG2b, IgG3, and IgM). If combinations of antibodies are used, the antibodies can be from the same subclass or from different subclasses. For example, the antibodies can be IgG1 antibodies. In some embodiments, the monoclonal antibody is humanized. Antibody fragments may include molecules such as Fab, scFv, F(ab′)2, and Fab′ molecules. Antibody derivatives include antibodies or fragments thereof having additions or substitutions, such as chimeric antibodies. Antibodies can be derived from human or animal sources, from hybridomas, through recombinant methods, or in any other way known to the art.

Binding partners other than antibodies or target analyte specific antibody fragments or derivatives can also be used in the present system and methods. For example, binding partners may be nucleic acids or nucleic-acid analogs, such as oligonucleotides or PNA probes. In one embodiment, aptamers can be used as specific binding partners. Aptamers are single-stranded DNA or RNA (ssDNA or ssRNA) molecules that can bind to pre-selected targets including proteins and peptides with high affinity and specificity. Other binding partners that can bind to target analyte to form pairs of receptor-ligand, enzyme-substrate, enzyme-inhibitor, and enzyme-cofactor pairs can also be used. Specific examples of such binding partner pairs include carbohydrate and lectin, biotin and avidin or streptavidin, folic acid and folate binding protein, vitamin B12 and intrinsic factor, Protein A and immunoglobulin, and Protein G and immunoglobulin. Also included are binding partners that form a covalent bond with the target analytes.

A polymer dye conjugate can comprise any known polymer dye conjugated to a binding partner using techniques known to those of skill in the art. In some embodiments, a polymer dye can be conjugated to a binding partner to form a polymer dye conjugate using the method of direct modification of core polymers described in US Published Application No. 2020/0190253, which is incorporated herein by reference in its entirety.

In some instances, a polymer dye can be conjugated to a binding partner to form a polymer dye conjugate using the method described in US Published Application No. 2019/0144601, which is incorporated herein by reference in its entirety. The method can be depicted as follows:

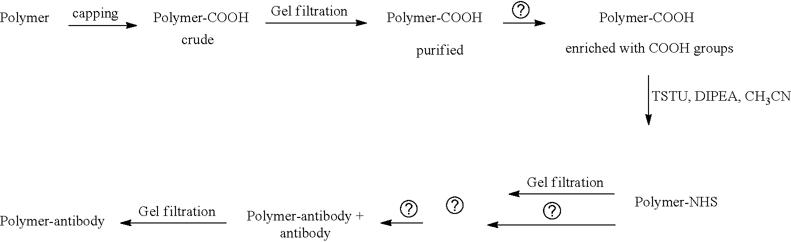

### Target Analyte

The disclosure also relates to a method for detecting a target analyte in a sample, wherein the target analyte comprises a target antigen and can be a substance, e.g., molecule, whose abundance/concentration is determined by some analytical procedure. The present invention is designed to detect the presence, and in some cases the quantity of specific target analytes. The term “target analyte” refers to a target molecule containing a target antigen to be detected in a biological sample, for example, peptides, proteins, polynucleotides, organic molecules, sugars and other carbohydrates, lipids, and small molecules. It is an important aspect of the disclosure that the target analytes are comprised in a liquid sample and are accessible, or made accessible at some point, to bind target analyte-specific binding partners of the instant invention. Target analytes may be found in a biological sample, such as a blood sample, a cell line development sample, a tissue culture sample, and the like.

The target analyte may be, for example, nucleic acids (DNA, RNA, mRNA, tRNA, or rRNA), peptides, polypeptides, proteins, lipids, ions, monosaccharides, oligosaccharides, polysaccharides, lipoproteins, glycoproteins, glycolipids, or fragments thereof. The target analyte can be a protein and can be, for example, a structural microfilament, microtubule, and intermediate filament proteins, organelle-specific markers, proteasomes, transmembrane proteins, surface receptors, nuclear pore proteins, protein/peptide translocases, protein folding chaperones, signaling scaffolds, ion channels and the like. The protein can be an activatable protein or a protein differentially expressed or activated in diseased or aberrant cells, including but not limited to transcription factors, DNA and/or RNA-binding and modifying proteins, nuclear import and export receptors, regulators of apoptosis or survival and the like.

Target analytes can be present and accessible on the surface of cells. Illustrative examples of useful analytes include, but are not limited to, the following: 1) specific cell surface macromolecules and antigens (including hormones, protein complexes, and molecules recognized by cell receptors) and 2) cellular proteins, DNA or RNA in permeabilized cells including abnormal DNA or RNA sequences or abnormal amounts of certain messenger RNA. Detection of these analytes may be particularly useful in situations where they are contained in and/or are identifiers of rare cells such as are found in the early stages of a variety of cancers.

In some examples, the target analyte may be a CD2, CD3, CD4, CD8, CD10, CD11c, CD14, CD15, CD16, CD19, CD20, CD22, CD25, CD27, CD38, CD45, CD45RA, CD56, CD62L, CD64, CD95, CD103, HLA-DR, IFN-alpha, IFN-beta, TNF-alpha, or ZAP-70, or other target analyte of interest.

### Non-Fluorescent Component of a Polymer Dye

The compositions according to the disclosure may include at least one, or at least two, non-fluorescent components of a polymer dye. The non-fluorescent component of a polymer dye may be selected from the group consisting of a monomeric component of a polymer dye, a non-fluorescent polymer dye, a photo-bleached polymer dye, and a polymer dye comprising a quenching moiety. In some embodiments, the non-fluorescent component of a polymer dye does not include a binding partner.

### Monomeric Component of a Polymer Dye

The compositions described herein include, among other components, a monomeric component of the polymer dyes (also referred herein as “monomers”, e.g., a monomer A and/or a monomer B) as described herein. The monomeric component of the polymer dye may be a water-soluble monomer.

The water-soluble monomer may be a monomeric unit comprising an aryl moiety or heteroaryl moiety, each optionally having a water-solubilizing moiety attached thereto. The water-solubilizing moiety may be one or more poly(ethylene glycol) moieties.

The monomeric component of the polymer dye may include dihydrophenanthrene (DHP)-based, fluorene-based and/or carbazole-based monomers.

For example, the water-soluble monomer may be a monomeric unit comprising an aryl moiety or heteroaryl moiety, for example, a monomer of the disclosure having a structure according to (II), (III), (IV), (Va), (Vb), (VIa), (VIb), (VIc), (VIIa), (VIIb), (VIIc), (VIId), (XXII), and/or (VVIII), for example, wherein both terminal ends of the monomers, represented by wavy lines, are independently or both a halogen atom, boronic ester or boronic acid, silyl, diazonium salt, triflate, acetyloxy, sulfonate, or phosphate which can undergo Pd or Nickel salt catalyzed polymerization reactions. DHP monomers of the disclosure can be as taught in US Published Application No. 2019/0144601 and U.S. Published Application No. 2020/0190253, both of which are incorporated herein by reference in theft entireties. The monomeric component of the polymer dye may be a dihydrophenanthrene (DHP)-based monomer having a chemical structure according to Formula (XXII):

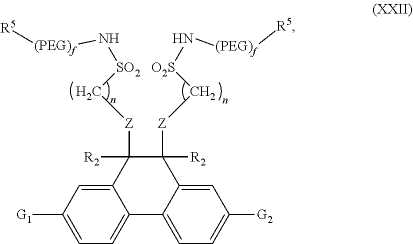

wherein:
each G¹, G² is independently selected from the group consisting of halo (F, Cl, Br, I), C₁-C₆ alkyl, and PEG; and each R², R⁴, R⁵, Z, n, and f are as previously defined herein.

The monomeric component of the polymer dye may be a fluorene-based monomer or carbazole-based monomer having a chemical structure according to Formula (XXIII):

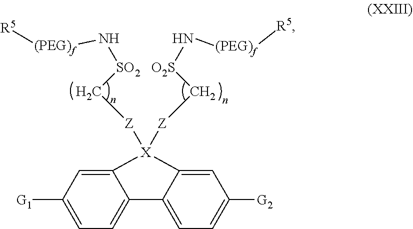

wherein:
each G¹, G² is independently selected from the group consisting of halo (F, Cl, Br, I), C₁-C₆alkyl, and PEG;
each X is C, N, or Si; and
each R², R⁴, R⁵, Z, n, and f are as previously defined herein.
In some embodiments, f is 1-50, 2-40, or 5-20.
In some embodiments, each n is 1-10, 2-5, or 3.
In some embodiments, each m is 11-12.

Examples of a monomeric component of a polymer dye include the following, which are referred to herein as exemplary “Monomer A” and exemplary “Monomer B”:

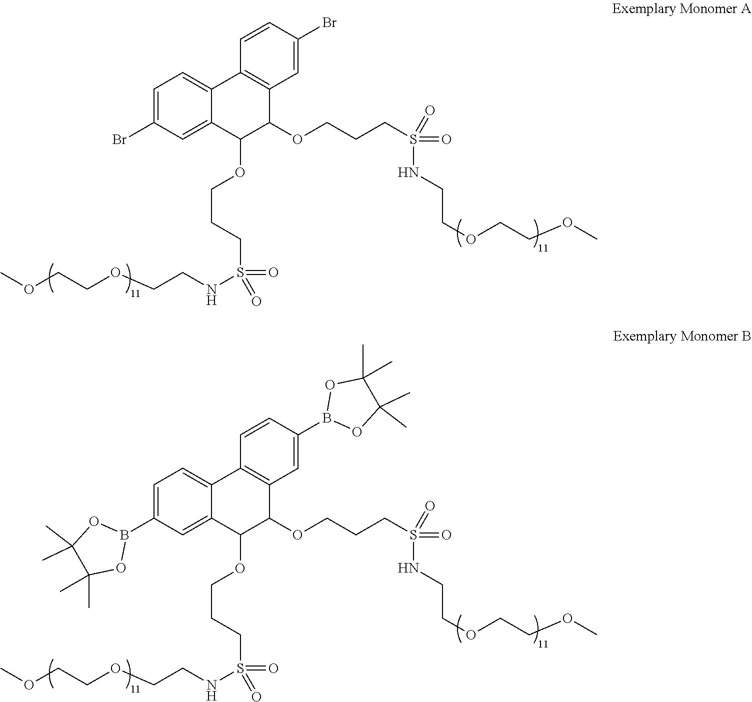

Various monomers A and B and derivatives thereof may be prepared by any appropriate methods as known in the art, for example, as provided in US Published Application No. 2019/0144601 or U.S. Published Application No. 2020/0190253, both of which are incorporated herein by reference in their entireties.

For example, 2,7-dibromo-trans-9,10-dihydrophenanthrene-9,10-diol (DHP-OH) can be prepared as follows. In a conical flask (2000 L), add about 26 g of NaBH₄ into a stirring water-ethanol mixture (120 mL+780 mL). To this solution, add about 24 g of 2,7-dibromophenanthrene,9, 10-dione portion-wise but quickly (in 5 min). The reaction mix allowed stirring for a day. The color of the solution changes from orange red to pale yellow to white by the end of the reaction. Stop the reaction and neutralize the reaction mixture with dil HCl acid. After the neutralization, filter the white precipitate and wash with excess water. Thus obtained white precipitate was washed with very cold (<−15° C,) ethanol (100 mL) and Methanol (100 mL).

DHP-O-alkyl-SO₃H can be prepared as follows. In a 2 neck round bottom flask, DHP-OH (3.6 g) and 18C6 (500 mg) were dissolved in 120 mL of THF. The solution was purged with nitrogen (20 min) and NaH (2 g) was added while nitrogen purging continues. The color of the solution changes from colorless to pale pink, dark pink, brown and dark green in 10-15 min. In another RB, 12 g of 1,3 propane sultone was dissolved in 20 of THF and nitrogen purged. This sultone solution was added to DHP-OH solution by addition funnel over a period of 20-30 minutes. The reaction was stirred at RT for 4-5 hrs. The solvents were evaporated, and dissolved the precipitate in water. Acetone was added to obtain white precipitate of DPS in the form of disodium salt. Filter the precipitate and redissolve in water (minimal amount) neutralize with HCl and precipitate again in acetone. Repeated precipitation (2-3 times) followed by centrifugation gives DPS as white solid.

DHP-O-alkyl-SO₂Cl can be prepared as follows. 5 g of DHP-O-alkyl-SO₃H was taken in a round bottom flask and mixed with 25 mL of DMF. To this about 10 L of SOCl₂ was added dropwise and the mixture allowed to stir for overnight. Next morning, reaction mixture was poured into 200 mL water and precipitate was filtered and dried.

DHP-sulfonamide PEG can be prepared as follows. DHP-O-alkyl-SO₂Cl was mixed with 2.2 equivalent of PEG amine in dichloromethane/TEA mixture. After 3 h sonication reaction the crude product was extracted in dichloromethane followed by column chromatography (silica gel, MeOH—CHCl₃).

Diboronic ester of DHP-sulfonamide PEG can be prepared as follows. The dibromo compound was mixed with DMSO under nitrogen and to this 3 equivalent of bispinacolatodiboron was added. The reagents were reacted with 12 equivalent of potassium acetate and 4 equivalent of Pd(dppf)Cl₂ catalyst for 5 hours at 80 deg. Reaction mixture cooled down and extracted with CHCl₃/water. The organic layer was concentrated and purified by column chromatography (silica gel, MeOH—CHCl₃).

Similarly, fluorene monomers of the disclosure can be made as described below. For example, FL-O-alkyl-SO₃H can be prepared as follows. In a 2 neck round bottom flask. 5 g of Fluorene was mixed with in 70 of DMSO. The solution was purged with nitrogen (20 min) and 50% NaOH (12 eq) was added while nitrogen purging continues. The color of the solution changes from colorless to dark brown. Propane sultone (3 eq) was weighed and dissolved in DMSO. This was added to the fluorene reaction mixture dropwise over a period of 5 minutes. The reaction was stirred at RT for 4-5 hrs. The solvents were evaporated, and dissolved the precipitate in water. Acetone was added to obtain white precipitate of DPS in the form of disodium salt. Filter the precipitate and redissolve in water (minimal amount) neutralize with HCl and precipitate again in acetone. Repeated precipitation (2-3 times) followed by centrifugation gives FL-OSO₃H as white solid.

FL-O-alkyl-SO₂Cl can be prepared as follows, 5 g of FL-O-alkyl-SO₃H was taken in a round bottom flask and mixed with 25 mL of DMF. To this about 10 mL of SOCl₂ was added dropwise and the mixture allowed to stir for overnight. Next morning, reaction mixture was poured into 200 mL water and precipitate was filtered and dried.

FL-sulfonamide PEG can be prepared as follows. FL-O-alkyl-SO₂Cl a was mixed with 2.2 equivalent of PEG amine in dichloromethane/TPA mixture. After 3 h sonication reaction the crude product was extracted in dichloromethane followed by column chromatography (silica gel, MeOH—CHCl₃).

Diboronic ester of FL-sulfonamide PEG can be prepared as follows. The dibromo compound was mixed with DMSO under nitrogen and to this 3 equivalent of bispinacolatodiboron was added. The reagents were reacted with 12 equivalent of potassium acetate and 4 equivalent of Pd(dppf)Cl₂ catalyst for 5 hours at 80 deg. Reaction mixture cooed down and extracted with CHCl₃/water. The organic layer was concentrated and purified by column chromatography (silica gel, MeOH—CHCl₂).

Addition of a monomeric component of a polymer dye such as an exemplary monomer A and/or an exemplary monomer B at 100-400 ug/test to a test staining buffer composition comprising a biological buffer and a nonionic surfactant, was found to be very efficient to reduce the non-specific interactions between polymer dye conjugates. The staining buffer composition of the disclosure may include from 10-500 mg/mL, 20-400 mg/mL, or 30-300 mg/mL monomeric component of a fluorescent dye. The working concentration staining buffer composition (1×) may include from 10-50 mg/mL, 20-40 mg/mL, or about 30 mg/mL of the monomeric component of a fluorescent polymer dye. The concentrated concentration (10×) staining buffer composition may include from 100-500 mg/mL, 200-400 mg/mL, or about 300 mg/mL of the monomeric component of a fluorescent polymer dye. The composition of the disclosure may include the monomeric component of the fluorescent polymer dye in a sufficient amount to supply from 100-400 ug/test, 200-400 ug/test, or about 300 ug/test.

### Quenched Polymer Dyes

The non-fluorescent component of a polymer dye may be a polymer dye comprising a quenching moiety, i.e., a quenched polymer dye (“quenched polymer”). Quenched polymers may comprise a polymer dye according to the disclosure comprising one or more, or a multiplicity of quenching moieties, for example, 1-50, 2-25, or 5-8 quenching moieties. In some embodiments, the quenched polymer exhibits a quantum yield (QY) no more than 0.1, or no more than 0.06, or no more than 0.056, no more than 0.05, no more than 0.02, or no more than 0.015ϕ. The quenched polymer may exhibit a fluorescent profile of less than 50 AFU, less than 40 AFU 30 AFU when excited with a 405 nm laser. In some embodiments, the quenched polymer dyes may exhibit >95% quenching of original maximum emission intensity, >98% quenching of original maximum emission intensity, up to 100% quenching compared to parent fluorescent polymer when excited at 405 nm.

In some embodiments, the quenched polymer dye emits ≤50 AFU at 450 nm (Slits ex/em are 6 nm/4 nm; 1 cm cuvette) for 10 micrograms/mL. In some embodiments, the quenched polymer dye exhibits <47 AFU at 10 ug/mL when excited with 405 nm laser, using AFU slits ex/em 6 nm/4 nm, in a Fluorimeter LS50B Perkin Elmer.

The quenching moiety can be a non-fluorescent quenching moiety. The non-fluorescent quenching moiety may be a dark quencher that is capable of absorbing excitation energy from a fluorophore and dissipating as heat.

The quenching moiety may be selected from, for example, DABCYL, DABSYL, DYQ425 Black Hole Quencher1 (BHQ1), QSY7, QSY9, QSY35, and TAMRA (carboxytetramethylrhodarmne) moieties. Quenching moieties are commercially available as, for example, N-hydroxysuccininide active esters (NHS esters) for conjugating to polymer dyes, e,g., for example, from Thermo Scientific (e.g., DYQ425; DyLight 4250 NHS ester). Other quenching moieties are available as, e.g., active esters as Dabcyl Q, Dabcyl plus, Anaspec 490Q, Dyomics 425Q, Dynomics 505Q, and so forth. Preferably, the quenching moiety is capable of quenching fluorescence emission within a range of from about 400 to 550 nm, about 480 to about 580 nm, or from about 500 to about 600 nm. Examples of quenching moieties may include, for example:

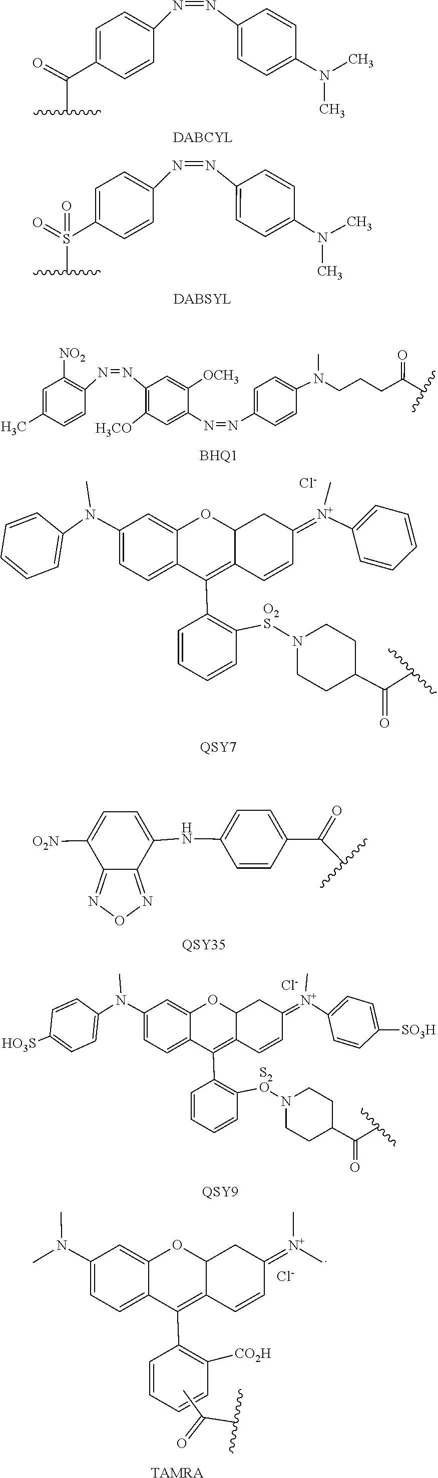

For example, the quenched polymer of the disclosure may utilize a polymer dye having a structure according to Formula (I):

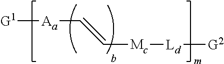

wherein each A is independently selected from the group consisting of an aromatic co-monomer and a heteroaromatic co-monomer; each L is a linker moiety; each M is independently selected from the group consisting of an aromatic co-monomer, a heteroaromatic co-monomer, a bandgap-modifying monomer, optionally substituted ethylene, and ethynylene; G¹ and G² are independently selected from an unmodified polymer terminus and a modified polymer terminus; a, c, and d independently define the mol % of each unit within the structure which each can be evenly or randomly repeated and where each a is a mol % from 10 to 100%, each c is a mol % from 0 to 90%, and each d is a mol % from 0 to 25%; each b is independently 0 or 1; and m is an integer from 1 to about 10,000, wherein the quenched polymer dye exhibits a quantum yield (QY) no more than 0.1, or no more than 0.06, or no more than 0.056.

In some embodiments, quenched polymer dye (I) exhibits a quantum yield (QY) no more than 0.1, or no more than 0.06, or no more than 0.056; optionally a fluorescent profile of less than 50 AFU, less than 40 AFU, or less than 30 AFU when excited with a 405 nm laser. In some embodiments, the quenched polymer dye I) may exhibit >95% quenching of original maximum emission intensity, >98% quenching of original maximum emission intensity, up to 100% quenching compared to parent fluorescent polymer when excited at 405 nm. The term “parent fluorescent polymer” refers to the polymer dye absent the quenching moieties.

For example, the quenched polymer of the disclosure may utilize dihydrophenanthrene (DHP), fluorene, carbazole and combinations of DHP, carbazole and fluorene monomers, for example, as shown in Formula (VIII):

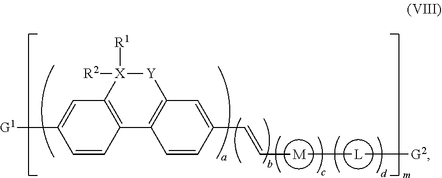

wherein G1, G2, R¹, R², X, Y, M, L, a, b, c, d, and m are as previously defined herein, wherein the polymer exhibits a quantum yield (QY) no more than 0.1, or no more than 0.06, or no more than 0.056. In some embodiments, quenched polymer dye (VIII) exhibits a fluorescent profile of less than 90 AFU, less than 50 AFU, less than 40 AFU, or less than 30 AFU when excited with a 405 nm laser. In some embodiments, the quenched polymer dye (VIII) may exhibit >95% quenching of original maximum emission intensity, >98% quenching of original maximum emission intensity, up to 100% quenching compared to parent fluorescent polymer when excited at 405 nm.

The quenched polymer according to Formula (VIII) may comprise 1-50, 2-25, or 5-8 quenching moieties at R¹, R², L, G¹ or G².

The quenched polymer may comprise a structure according to Formula (XX), wherein the quenching moiety is attached at L:

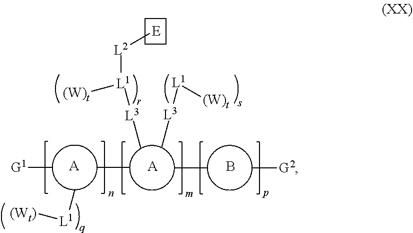

wherein each A, B, E, G¹, G², L¹, L², L³, W, n, m, p, q, r, s, t is as previously defined herein; and

A and B are distributed randomly or non-randomly in the conjugated polymer, wherein the polymer exhibits a quantum yield (QY) no more than 0.1, or no more than 0.06, or no more than 0.056. In some embodiments, quenched polymer dye (XX) exhibits a fluorescent profile of less than 90 AFU, less than 50 AFU, less than 40 AFU, or less than 30 AFU when excited with a 405 nm laser. In some embodiments, the quenched polymer dye (XX) may exhibit >95% quenching of original maximum emission intensity, >98% quenching of original maximum emission intensity, up to 100% quenching compared to parent fluorescent polymer when excited at 405 nm.

In some embodiments, each E may be an independently selected chromophore, functional moiety, or quenching moiety, wherein at least one or more E, or at least two or more E is a quenching moiety.

In some examples, the quenched polymer dyes of the disclosure may utilize a polymer dye having a structure according to Formula (I), (XVIII)-(XIV), (XVIII), (XIX), (XX), and/or (XXIV).

In some embodiments, the quenched polymer may comprise 2-20, 3-15, or 5-8 quenching moieties. In some embodiments, m=2-20, 3-15, or 5-8.

In some embodiments, the quenching moiety may be selected from a Dabcyl, Dabsyl, BHQ1, DYQ425, DYQ505, QSY7, QSY9, QSY35, or TAMRA quenching moieties.

In some embodiments, the quenched polymer does not include a binding partner.

The quenched polymers may be prepared according to any appropriate method, for example, wherein the quenching moiety is obtained commercially in the form of an active ester, such as an NHS-ester, and exposed to the polymer dye according to methods of the disclosure.

The staining buffer of the disclosure may include 0.2-15 mg/mL, 0.3-12 mg/mL, or 0.5-10 mg/mL of the quenched polymer. In some embodiments, a working concentration (1×) staining buffer composition of the disclosure may include from 0.2-2.0 mg/mL, 0.3-1.5 mg/mL, 0.5-1.2 mg/mL, or about 1 mg/mL of the quenched polymer dye. In some embodiments, a concentrated staining buffer composition (10×) may include 3-15 mg/mL, 5-12 mg/mL, or about 10 mg/mL of the quenched polymer. The staining buffer composition of the disclosure may include the quenched polymer dye in a sufficient amount to supply from 2-20 ug/test, 3-15 ug/test, or about 10 ug/test.

### Photo-Bleached Polymer Dyes

The compositions described herein may comprise at least one photo-bleached polymer dye having a structure according to the disclosure. For example, the photo-bleached polymer dye may comprise a structure according to any of Formulas I, VIII-XIV, XVIII, XIX and/or XX according to the disclosure. Such compounds have been described previously in Published PCT Appl. No. WO2017/180998 and Published US Appl. No. 2020/0190253 A1, which are incorporated by reference as if fully set forth herein in its entirety.

The term “photo-bleached dye” refers to a dye originally comprising a fluorophore that has undergone high-intensity illumination such that it can no longer fluoresce, or exhibits quantum yield (QY) of no more than 0.1. In some embodiments, the photo-bleached polymer exhibits a quantum yield (QY) no more than 0.1, no more than 0.06, no more than 0.056, no more than 0.02, or no more than 0.015 ϕ. In some embodiments, the photo-bleached polymer dye exhibits less than about 50 arbitrary units of fluorescence (AFU) when excited by 405 nm laser. In some embodiments, the photo-bleached polymer dye may exhibit >95% quenching of original maximum emission intensity, >98% quenching of original maximum emission intensity, up to 100% quenching compared to parent fluorescent polymer when excited at 405 nm. The term “parent fluorescent polymer” refers to the polymer dye prior to photo-bleaching.

A fluorophore can undergo the fluorescence process repeatedly. This means that the fluorophore molecule can theoretically generate a signal multiple times. In reality, the fluorophore's structural instability during its excited lifetime may make it susceptible to degradation. High-intensity illumination can cause the fluorophore to change its structure so that it can no longer fluoresce and this is called photo-bleaching.

As used herein, the term “photo-bleached polymer dye” generally refers to polymer dyes, for example, violet-excitable polymer dyes, that, after photo-bleaching, exhibit a quantum yield (QY) no more than 0.1, or no more than 0.06, or no more than 0.056. in some embodiments, photo-bleached dye exhibits less than about 50 arbitrary units of fluorescence (AFU), less than about 45 AFU, less than about 40 AFU, less than about 35 AFU, less than about 30 AFU, less than about 25 AFU, less than about 20 AFU, less than about 15 AFU, less than about 10 AFU, less than about 5 AFU, less than about 1 AFU, about 1 AFU to about 50 AFU, about 5 AFU to about 25 AFU, about 20 AFU to about 40 AFU, about 15 AFU to about 30 AFU, about 15 AFU to about 40 AFU or about 20 AFU to about 36 AFU.

In some embodiments, the photo-bleached dye emits ≤50 AFU at 450 nm (Slits ex/em are 6 nm/4 nm; 1 cm cuvette) for 10 micrograms/mL. In some embodiments, the photo-bleached polymer dye exhibits <47 AFU at 10 ug/mL when excited with 405 nm laser, using AFU slits ex/em 6 nm/4 nm, in a Fluorimeter LS50B Perkin Elmer. For example, the photo-bleached polymer dye may be a violet dye originally comprising a fluorophore that has undergone high-intensity UV illumination such that it can no longer fluoresce, or exhibits a quantum yield (QY) no more than 0.1, or no more than 0.06, or no more than 0.056; optionally less than about 50 arbitrary fluorescence units (AFU) when excited by a 405 nm laser. In some embodiments, the photo-bleached polymer dye may exhibit >95% quenching of original maximum emission intensity, >98% quenching of original maximum emission intensity, up to 100% quenching compared to parent fluorescent polymer when excited at 405 nm.

For example, the photo-bleached polymer dyes of the disclosure may utilize a polymer dye having a structure according to Formula (I):

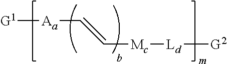

wherein each A is independently selected from the group consisting of an aromatic co-monomer and a heteroaromatic co-monomer; each L is a linker moiety; each M is independently selected from the group consisting of an aromatic co-monomer, a heteroaromatic co-monomer, a bandgap-modifying monomer, optionally substituted ethylene, and ethynylene; G¹ and G² are independently selected from an unmodified polymer terminus and a modified polymer terminus; a, c, and d independently define the mol % of each unit within the structure which each can be evenly or randomly repeated and where each a is a mol % from 10 to 100%, each c is a mol % from 0 to 90%, and each d is a mol % from 0 to 25%; each b is independently 0 or 1; and m is an integer from 1 to about 10,000, wherein the photo-bleached polymer dye is prepared by exposing a fluorescent polymer dye according to formula (I) to UV light illumination such that it exhibits a quantum yield (QY) no more than 0.1, or no more than 0.06, or no more than 0.056; optionally wherein it exhibits less than about 50 arbitrary units of fluorescence (AFU) when excited by a 405 nm laser; and or optionally exhibits >95% quenching of original maximum emission intensity, >98% quenching of original maximum emission intensity, up to 100% quenching compared to parent fluorescent polymer when excited at 405 nm.

For example, the photo-bleached polymer dyes of the disclosure may utilize dihydrophenanthrene (DIP), fluorene, and combinations of DHP and fluorene monomers, for example, as shown in Formula (VIII):

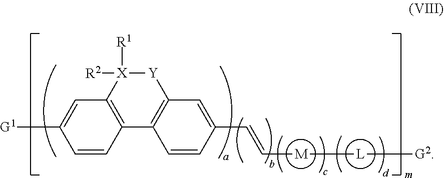

wherein G1, G2, R1, R2, X, Y, M, L, a, b, c, d, and m are described herein above, wherein the photo-bleached polymer dye is prepared by exposing a fluorescent polymer dye according to formula (VIII) to UV light illumination such that it exhibits a quantum yield (QY) no more than 0.1, or no more than 0.06, or no more than 0.056; optionally less than about 50 arbitrary units of fluorescence (AFU), when it is excited by the 405 nm laser; and or optionally exhibits >95% quenching of original maximum emission intensity, >98% quenching of original maximum emission intensity, up to 100% quenching compared to parent fluorescent polymer when excited at 405 nm.

For example, the photo-bleached polymer dyes of the disclosure may utilize a polymer dye having a structure according to Formula (XX):

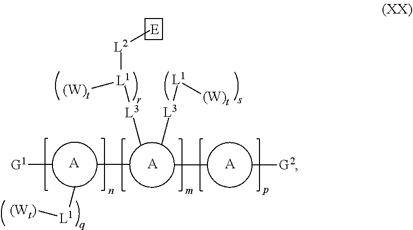

wherein each A, B, E, G1, G2, L¹, L2, L³, W, n, m, p, q, r, s, t is as previously defined herein; and
A and B are distributed randomly or non-randomly in the conjugated polymer, wherein the photo-bleached polymer dye is prepared by exposing a fluorescent polymer dye according to formula (XX) to UV light illumination such that it exhibits quantum yield (QY) of no more than 0.1, or no more than 0.06, or no more than 0.056; optionally less than about 50 arbitrary units of fluorescence (AFU), when it is excited by the 405 nm laser. In some embodiments, the photo-bleached polymer dye may exhibit >95% quenching of original maximum emission intensity, >98% quenching of original maximum emission intensity, up to 100% quenching compared to parent fluorescent polymer when excited at 405 nm.

For example, the photo-bleached polymer dyes of the disclosure may utilize a polymer dye having a structure according to Formula (XVIII), wherein the photo-bleached polymer dye is prepared by exposing a fluorescent polymer dye according to formula (XVIII) to UV light illumination such that it exhibits a quantum yield (QY) no more than 0.1, or no more than 0.06, or no more than 0.056; optionally exhibits less than about 50 arbitrary units of fluorescence (AFU), when it is excited by the 405 nm laser; optionally exhibits >95% quenching of original maximum emission intensity, >98% quenching of original maximum emission intensity, up to 100% quenching compared to parent fluorescent polymer when excited at 405 nm.

For example, the photo-bleached polymer dyes of the disclosure may be prepared from a fluorescent polymer dye such as a violet-excitable fluorescent polymer dye, for example, SuperNova™ (“SN”) v428 (Beckman Coulter, Inc.) is a fluorescent polymer dye optimally excited by the violet laser (405 nm), wherein the photo-bleached polymer dye is prepared by exposing a fluorescent polymer dye according to formula (I) to UV light illumination such that it exhibits less than about 50 arbitrary units of fluorescence (AFU). In some embodiments, the photo-bleached dye does not include a binding partner.

The composition of the disclosure may include a photo-bleached dye in a working concentration (1×) of from about 0.2-0.8 mg/mL, 0.3-0.7 mg/mL, or about 0.5 mg/mL. The composition of the disclosure may include a photo-bleached dye in a concentrated composition (10×) of about 2-8 mg/mL, 3-7 mg/mL, or about 5 mg/mL. The composition of the disclosure may include a photo-bleached dye in a range of from about 0.1 to about 10 mg/mL, 0.2-8 mg/mL, 0.3-7 mg/mL, or 0.5-5 mg/mL. The composition of the disclosure may include a photo-bleached dye in a sufficient amount to supply 2-8 ug/test, 3-7 ug/test, or about 5 ug/test.

### Nonionic Surfactants

The composition may comprise one or more nonionic surfactants. A sufficient amount of the nonionic surfactant can be included to prevent aggregation of polymer dye conjugates. Non-limiting examples of nonionic surfactants includes poloxamer surfactants, such as PLURONIC™F-68 (PF-68), polysorbates, including TWEEN® 20 and TWEEN® 80, and ether-linked nonionic surfactants such as, for example, polyoxyethylene glycol alkyl ether (BRIJ), a polyoxyethylene glycol octylphenol ether (TRITON), or a polyoxyethylene nonylphenyl ether (IGEPAL) surfactant. In some embodiments, the surfactant is a poloxamer nonionic surfactant.

The term poloxamer nonionic surfactant refers to a polyethylene oxide-polypropylene oxide-polyethylene oxide (PEG-PPG-PEG) nonionic triblock copolymer. The term poloxamer nonionic surfactant encompasses PLURONIC® nonionic surfactants. Examples of PLURONIC® surfactants include, for example, PLURONIC® F68, F77, F87, F98, F108, F127, P103, P104, P105, and P123.

In some embodiments, the surfactant is a polyoxypropylene-containing surfactant such as a poloxamer surfactant. Poloxamer surfactants include nonionic triblock copolymers such as polyoxyethylene oxide-polyoxypropylene oxide-polyoxyethylene oxide (PEO-PPO-PEO) characterized by a central hydrophobic chain of polyoxypropylene (poly(propylene oxide)) flanked by two hydrophilic chains of polyoxyethylene (poly(ethylene oxide)). Exemplary nonionic triblock copolymers may comprise a structure according to Formula (XXI),

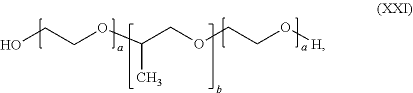

wherein each a is independently an integer independently in the range of 2-130, and b is an integer in the range of 15-67. In some embodiments, a is in the range of 50-100 and b is in the range of 20-40. In some embodiments, a is in the range of 70-90 and b is in the range of 25-30. The nonionic surfactant may be poloxamer 188. Non-limiting examples of poloxamers may include poloxamer 188, also known as Pluronic F-68, or KOLLIPHOR® P188, e.g., having a=80 and b=27. Other poloxamers include poloxamer 338, also known as Synperonic™ PE/F108, poloxamer 407, also known as Synperonic™ PE/F127, poloxamer 331, also known as Synperonic™ PE/L101.

The term “PLURONIC® F68,” “Pluronic F-68”, or “PF-68”, also known as poloxamer 188, refers to poly(ethylene glycol)-block-poly(propylene glycol)-block poly(ethylene glycol) copolymer with an average molecular weight, avg. Mn, of 8350-8400.

The term “PLURONIC® F127” also known as poloxamer 407 refers to a triblock copolymer consisting of a central hydrophobic block of polypropylene glycol flanked by two hydrophilic blocks of polyethylene glycol (PEG). The approximate lengths of the two PEG blocks is 101 repeat units, while the approximate length of the propylene glycol block is 56 repeat units. This is also known by the Croda trade name Synperonic PE/F 127, of avg. 12,600 g/mol.

The term “PLURONIC® F108” refers to poly(ethylene glycol)-block-poly(propylene glycol)-block poly(ethylene glycol), avg. Mn ˜14,600.

The term “PLURONIC® P103” refers to poly(ethylene glycol)-block-poly(propylene glycol)-block poly(ethylene glycol), of avg. Mw ˜4,950.

The term “PLURONIC® P104” refers to poly(ethylene glycol)-block-poly(propylene glycol)-block poly(ethylene glycol), of avg. Mw ˜5,900.

The term “PLURONIC® P123” refers to poly(ethylene glycol)-block-poly(propylene glycol)-block poly(ethylene glycol), of avg. Mn about ˜5,800.

Because the lengths of the polymer blocks can be customized, many different poloxamers exist that have slightly different properties. Poloxamer copolymers are commonly named with the letter “P” (for poloxamer) followed by three digits, the first two digits×100 give the approximate molecular mass of the polyoxypropylene core, and the last digit×10 gives the percentage polyoxyethylene content (e.g., P407=Poloxamer with a polyoxypropylene molecular mass of 4,000 g/mol and a 70% polyoxyethylene content). For the Pluronic and Synperonic poloxamer tradenames, coding of these copolymers starts with a letter to define its physical form at room temperature (L=liquid, P=paste, F=flake (solid)) followed by two or three digits. The first digit (two digits in a three-digit number) in the numerical designation, multiplied by 300, indicates the approximate molecular weight of the hydrophobic chain; and the last digit×10 gives the percentage polyoxyethylene content (e.g., F-68 indicates a polyoxypropylene molecular mass of 1,800 g/mol and a 80% polyoxyethylene content). An exemplary poloxamer surfactant includes, but is not limited to, Pluronic F-68. PF-68 is a nonionic triblock copolymer polyoxyethylene oxide-polyoxypropylene oxide-polyoxyethylene oxide (PEO-PPO-PEO). The concentration of the surfactant used can be determined empirically (i.e., titrated such that precipitation of the conjugates does not occur). In some embodiments, the staining buffer composition may include a nonionic surfactant such as a poloxamer surfactant. The nonionic surfactant may be Pluronic F-68 (poloxamer 188). The nonionic surfactant may be present in the staining buffer composition at a working concentration (1×) of 0.01-10%, 0.02-8%, 0.05-7%, 0.1-5%, 0.2-2%, 0.1-0.4% or about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.5%, 2%, 3%, 4%, 5%, 6%, 7%, 8%, 9% or 10% (wt/vol), or any value in between. The nonionic surfactant may be present in a concentrated staining buffer composition (10×) at 0.1-40%, 0.2-30%, 0.5-25%, or 10-20% (wt/vol). In some embodiments, a staining buffer composition of the disclosure may comprise 0.01-40%, 0.01-20%, 0.02-10% (wt/vol) nonionic surfactant.

### Biological Buffers

The term “biological buffer” refers to a physiologically compatible aqueous solution comprising one or more biological buffering agents which in a cell-free system maintains pH in the biological range of pH 6-8, 6.5-8, or 7-8. in certain embodiments, the biological buffering agents may include one or more of N-(2-acetamido)-aminoethanesulfonic acid (ACES), acetate, N-(2-acetamido)-iminodiacetic acid (ADA), 2-aminoethanesulfonic acid (AES), ammonia, 2-amino-2-methyl-1-propanol (AMP), 2-amino-2-methyl-1,3-propanediol (AMPD), N-(1,1-dimethyl-2-hydroxyethyl)-3-amino-2-hydroxypropanesulfonic acid (AMPSO), N,N-bis-(2-hydroxyethyl)-2-aminoethanesulfonic acid (BES), bicarbonate, N,N′-bis-(2-hydroxyethyl)-glycine, [Bis-(2-hydroxyethyl)-imino]-tris-(hydroxymethylmethane) (BIS-Tris), 1,3-Bis[tris(hydroxymethyl)-methylamino]propane (BIS-Tris-propane), boric acid, dimethylarsinic acid, 3-(Cyclohexylamino)-propanesulfonic acid (CAPS), 3-(cyclohexylamino)-2-hydroxy-1-propanesulfonic acid (CAPSO), carbonate, cyclohexylaminoethanesulfonic acid (CHES), citrate, 3-[N-Bis(hydroxyethyl)amino]-2-hydroxypropanesulfonic acid (DIPSO), formate, glycine, glycylglycine, N-(2-Hydroxyethyl)-piperazine-N′-ethanesulfonic acid (HEPES), lactate, N-(2-Hydroxyethyl)-piperazine-N′-3-propanesulfonic acid (HEPPS, EPPS), N-(2-Hydroxyethyl)-piperazine-N′-2-hydroxypropanesulfonic acid (HEPPSO), imidazole, malate, maleate, 2-(N-Morpholino)-ethanesulfonic acid (MES), 3-(N-Morpholino)-propanesulfonic acid (MOPS), 3-(N-Morpholino)-2-hydroxypropanesulfonic acid (MOPSO), phosphate, Piperazine-N,N′-bis(2-ethanesulfonic acid) (PIPES), Piperazine-N,N′-bis(2-hydroxypropanesulfonic acid) (POPSO), pyridine, polyvinylpyrrolidone (PVP), succinate, 3-{[Tris(hydroxymethyl)-methyl]-amino}-propanesulfonic acid (TAPS), 3-[N-Tris(hydroxymethyl)-methylamino]-2-hydroxypropanesulfonic acid (TAPSO), 2-Aminoethanesulfonic acid, AES (Taurine), trehalose, triethanolamine (TEA), 2-[Tris(hydroxymethyl)-methylamino]-ethanesulfonic acid (TES), N-[Tris(hydroxymethyl)-methyl]-glycine (tricine), Tris(hydroxymethyl)-aminomethane (Tris), glyceraldehydes, mannose, glucosamine, mannoheptulose, sorbose-6-phosphate, trehalose-6-phosphate, iodoacetates, sodium citrate, sodium acetate, sodium phosphate, sodium tartrate, sodium succinate, sodium maleate, magnesium acetate, magnesium citrate, potassium phosphate, magnesium phosphate, ammonium acetate, ammonium citrate, ammonium phosphate, among other buffers. Representative buffering agents may include salts of organic acid salts, such as citric acid, ascorbic acid, gluconic acid, carbonic acid, tartaric acid, succinic acid, acetic acid, or phthalic acid; Tris tromethamine hydrochloride, or phosphate. Specific examples of conventional biological buffers may include phosphate-buffered saline (PBS), N-2-Hydroxyethylpiperazine-N′-2-hydroxypropanesulfonic acid (HEPES), 2-(N-morpholino)ethanesulfonic acid (MES), 3-(N-Morpholino) propanesulfonic acid (MOPS), 2-([2-Hydroxy-1,1-bis(hydroxymethyl)ethyl]amino)ethanesulfonic acid (TES), 3-[N-tris(Hydroxy-methyl) ethylamino]-2-hydroxyethyl]-1-piperazinepropanesulfonic acid (EPPS), Tris[hydroxymethyl]-aminomethane (THAM), 1,4-piperazinediethanesulfonic acid (PIPES), and Tris[hydroxymethyl]methyl aminomethane (TRIS) buffers. Conventional biological buffers may have a pK in the physiological range and function most efficiently in this range. The biological buffer may be in aqueous solution at a concentration of, for example, 10-100 mM, or 5-25 mM.

The term “PBS” refers to phosphate buffered saline which is an aqueous buffer which may contain sodium chloride, disodium hydrogen phosphate, potassium chloride, and/or potassium dihydrogen phosphate. For example, PBS may contain milliQ water or deionized water and 137 mM NaCl, 2.7 mM KCl, 10 mM Na₂HPO₄, 1.8 mM KH₂PO₄. The pH may be about pH 7.0-7.4. The PBS may or may not be preserved with an azide such as sodium azide. The PBS may be an isotonic solution. The buffer may be a PBA buffer. The PBA buffer may comprise PBS, BSA, and sodium azide. The PBA buffer may comprise 1×PBS, about 2 mg/mL BSA, and about 0.1% sodium azide.

### Additional Components

The compositions of the disclosure can be used as staining buffer compositions, for example, in flow cytometry sample analysis and, as such, can comprise additional components, including, but not limited to, one or more of any suitable carriers, stabilizers, salts, chelating agents (e.g., EDTA), colorants, or preservatives. The compositions can also comprise an additional one or more surfactants (e.g., ionic surfactants, and zwitterionic surfactants).

The term “protein stabilizer” refers to a protein that serves to reduce non-specific binding, for example, to reduce cell-cell interactions, or to help prevent non-specific binding between an antibody and a non-target molecule. The compositions according to the disclosure may include a protein stabilizer. The protein stabilizer may be selected from one or more of the group consisting of a serum albumin, for example, a bovine serum albumin (BSA), a casein, or a gelatin. The protein stabilizer may be a BSA. In some embodiments, the protein stabilizer is present at 0.1-100 mg/mL, 0.2-50 mg/mL, 1-20 mg/mL, or 1-10 mg/mL. The protein stabilizer may be present in the working composition (1×) of the disclosure at a concentration of from 0.1-10 mg/mL, 0.5-5 mg/mL, 1-3 mg/mL or about 2 mg/mL. The protein stabilizer may be present in the concentrated composition (10×) of the disclosure at a concentration of from 1-100 mg/mL, 5-50 mg/mL, 10-30 mg/mL or about 20 mg/mL.

The carrier can be an aqueous solution, such as water, saline, alcohol, or biological buffer, such as PBS, Hank's solution, Ringer's solution, or physiological saline buffer.

The carrier can include formulation agents, such as suspending agents, stabilizing agents and/or dispersing agents.

For example, the staining buffer composition may contain a carrier such as water, or a solvent such as, e.g., DMSO as a solubilizing agent.

The compositions can also include an appropriate biological buffer and/or pH adjusting agent, and typically the buffer is a salt prepared from an organic add or base.

The compositions of the disclosure may include any appropriate preservative. The preservative may be an antioxidant, biocide, or antimicrobial agent. The preservative may be an inorganic salt. For example, the preservative may be sodium azide, 2-chloroacetamide, 2-methylisothiazolinone, salicylic acid, ProClin™, Kathon™ CG, 5-chloro-2-methyl-4-isothiazolin-3-one, or 2-methyl-4-isothiazolin-3-one. The preservative may be present in the composition of the disclosure at 0.01-0.5%, 0.05-0.3%, or about 0.1%.

The compositions of the disclosure may include additional surfactants. Suitable additional surfactants that can optionally be used according to the methods described herein may include zwitterionic surfactants, such as betaines such alkyl betaines, alkylamidobetaines, amidazoliniumbetaines, sulfabetaines (INCI Sultaines), as well as a phosphobetaines. Examples of suitable zwitterionic surfactants include surfactants of the general formula R^{1′}[CO—X(CH₂)ⱼ]_{g}—N⁺(R^{2′})(R³)—(CH₂)_{f}—[CH(OH)CH₂]ₕ—Y⁻, wherein R^{1′} is a saturated or unsaturated C₆₋₂₂ alkyl, such as a C₈₋₁₈ alkyl, a saturated C₁₀₋₁₆ alkyl or a saturated C₁₂₋₁₄ alkyl; X is NH, NR^{4′}, wherein R^{4′} is C₁₋₄ alkyl, O or S; j is an integer from 1 to 10, such as from 2 to 5 and 3; g is 0 or 1, R^{2 ′} and R^{3′} are each, independently, a C₁₋₄ alkyl, optionally hydroxy substituted by a hydroxyethyl group or a methyl; f is an integer from 1 to 4, such as 1, 2 or 3; h is 0 or 1; and Y is COO, SO₃, OPO(OR^{5′})O or P(O)(OR^{5′})O, wherein R^{5′} is H or C₁₋₄ alkyl.

Examples of suitable zwitterionic surfactants include alkyl betaines, such as those of the formula:

R^{1′}—N⁺(CH₃)₂—CH₂COO⁻;

R^{1′}—CO—NH(CH₂)₃-N⁺(CH₃)₂—CH₂COO⁻;

R^{1′}—N⁺(CH₃)₂-CH₂CH(OH)CH₂SO₃⁻; and

R^{1′}—CO—NH—(CH₂)₃—⁺(CH₃)—CH₂CH(OH)CH₂SO₃⁻.

Examples of suitable betaines and sulfobetaines are the following (designated in accordance with INCI): almondamidopropyl betaine, apricotamidopropyl betaine, avocadamidopropyl betaine, babassuamidopropyl betaine, behenamidopropyl betaine, behenyl betaine, canolamidopropyl betaine, caprylicapramidopropyl betaine, carnitine, cetyl betaine, cocamidoethyl betaine, cocamidopropyl betaine, cocamidopropyl hydroxysultaine, coco betaine, coco hydroxysultaine, coco/oleamidopropyl betaine, coco decyl betaine, dihydroxyethyl oleyl glycinate, dihydroxyethyl soy glycinate, dihydroxyethyl stearyl glycinate, dihydroxyethyl tallow glycinate, dimethicone propyl of PG-betaine, drucamidopropyl hydroxysultaine, hydrogenated tallow betaine, isostearamidopropyl betaine, lauramidopropyl betaine, lauryl betaine, lauryl hydroxysultaine, lauryl sultaine, milk amidopropyl betaine, milkamidopropyl betaine, myristamidopropyl betaine, myristyl betaine, oleamidopropyl betaine, oleamidopropyl hydroxysultaine, oleyl betaine, olivamidopropyl betaine, palmamidopropyl betaine, palmitamidopropyl betaine, palmitoyl carnitine, palm kernel amidopropyl betaine, polytetrafluoroethylene acetoxypropyl betaine, ricinoleamidopropyl betaine, sesamidopropyl betaine, soyamidopropyl betaine, stearamidopropyl betaine, stearyl betaine, tallowamidopropyl betaine, tallowamidopropyl hydroxysultaine, tallow betaine, tallow dihydroxyethyl betaine, undecylenamidopropyl betaine and wheat germ amidopropylbetaine.

For example, coconut dimethyl betaine is commercially available from Seppic under the trade name of AMONYL 265®; and lauryl betaine is commercially available from Sigma-Aldrich under the trade name EMPIGEN BB®. A further example betaine is lauryl-imino-dipropionate commercially available from Rhodia under the trade name MIRATAINE H2C-HA®. Presence of optional zwitterionic surfactant in staining buffer composition may decrease non-specific binding in a biological sample, for example, may decrease non-specific binding to monocytes. The optional zwitterionic surfactant may be present in the composition at 0-0.5%, 0.05-0.3%.

### Compositions

Staining buffer compositions are provided for decreasing polymer-polymer interactions between polymer dye conjugates and decreasing dye conjugate precipitation in a biological sample. Staining buffer compositions are provided for decreasing polymer-polymer interactions between polymer dye conjugates in a multi-color panel comprising two or more polymer dye conjugates.

The compositions according to the disclosure may be added to a mixture of dye conjugates comprising one or more, two or more, or three or more polymer dye conjugates prior to, concurrently with, or after adding to a biological sample for decreasing, substantially decreasing and/or preventing non-specific binding between dye conjugates such as polymer-polymer interactions. The mixture of dye conjugates may include one or more, two or more, or three or more polymer dye conjugates and optionally one or more, two or more, three or more conventional fluorescent dye conjugates such as, for example, fluorescein, coumarin, cyanine, rhodamine dye conjugates, for example, FITC (fluorescein isothiocyanate), PE (phycoerythrin), ECD (phycoerythrin-Texas Rede-X), PC5 (phycoerythrin-cyanine 5.5), PC5.5 (phycoerythrin-cyanine 5.5), PC7 (phycoerythrin-cyanine 7), APC (allophycocyanine), AA700, AA750, PBE, Alexa Fluor® 488 (AF488), AF532, AF647, AF700, AF750, Atlantis Bioscience CF®350 dye, CF®405S, CF®405, CF®405L, CF®430, CF®440, CF®450, CF®488A, CF®514, AAT Bioquest iFluor™ 488, iFluor™ 350, iFluor™ 405, mFluor™ Blue 570, mFluor™ Blue 580, mFluor™ Blue 590, mFluor™ Blue 620, mFluor™ Blue 630, mFluor™ Blue 660, ThermoFisher Scientific NovaFluor Blue 510, NovaFluor Blue 530, NovaFluor Blue 555, NovaFluor Blue 585, NovaFluor Blue 610/30S, NovaFluor Blue 660/40S, NovaFluor Blue 660/120S, BioLegend® Kiravia Blue 520™, KrO dye conjugates, and the like. Other dyes or dye conjugates may include a Super Bright polymer dye (Invitrogen, ThermoFisher Scientific). Super Bright dyes may be excited by the violet laser (405 nm). The Super Bright dye may be Super Bright 436 (excitation max 414 nm, emission max 436 nm, 450/50 bandpass filter), Super Bright 600 (emission max 600 nm, 610/20 bandpass filter), Super Bright 645 (emission max 645 nm, 660/20 bandpass filter), or Super Bright 702 (emission max 702 nm, 710/50 bandpass filter).

The disclosure provides compositions comprising at least one non-fluorescent component of a first polymer dye; and a biological buffer. The composition may comprise at least one non-fluorescent component of a first polymer dye; a biological buffer; and a nonionic surfactant. The nonionic surfactant may be a poloxamer nonionic surfactant. For example, the composition may include a non-fluorescent component of a first polymer dye selected from one or more of a monomeric component of a polymer dye, a photo-bleached polymer dye, and a polymer dye comprising a quenching moiety.

Staining buffer compositions are provided including a monomeric component a polymer dye, a photo-bleached polymer dye, and a biological buffer. Staining buffer compositions are provided including a monomeric component a polymer dye, a photo-bleached polymer dye, a nonionic surfactant, and a biological buffer. In some embodiments, a staining buffer composition is provided comprising a working concentration (1×) of 10-40 mg/mL monomer A, 0.2-1.5 mg/mL photo-bleached dye, and a biological buffer, optionally with a protein stabilizer and a preservative. In some embodiments, a staining buffer composition is provided comprising a working concentration (1×) of 10-40 mg/mL monomer A, 0.2-1.5 mg/mL photo-bleached dye, 0.01-10% (wt/vol) nonionic surfactant and a biological buffer, optionally with a protein stabilizer and a preservative. In some embodiments, staining buffer composition is provided comprising a working concentration (1×) of 20-40 mg/mL monomer A, 0.2-0.8 mg/mL photo-bleached dye, 0.01-4% (wt/vol) nonionic surfactant and a biological buffer, optionally with a protein stabilizer and a preservative.

Staining buffer compositions are provided including a monomeric component of a polymer dye, a nonionic surfactant, and a biological buffer. In some embodiments, a staining buffer composition is provided comprising a working concentration (1×) of 10-40 mg/mL monomeric component of a polymer dye, 0.01-4% (wt/vol), nonionic surfactant and a biological buffer, optionally with a protein stabilizer and a preservative.

Staining buffer compositions are provided including a polymer dye comprising a quenching moiety, a biological buffer, and a nonionic surfactant. Staining buffer compositions are provided including a monomeric component of a polymer dye, a polymer dye comprising a quenching moiety, a biological buffer, and a nonionic surfactant. Optionally, the composition may include a protein stabilizer. Optionally, the composition may include a preservative.

In some embodiments, a staining buffer composition is provided comprising 10-40 mg/mL monomer A, 0.2-1.5 mg/mL quenched polymer, 0.01-4% (wt/vol) nonionic surfactant and a biological buffer, optionally with a protein stabilizer and a preservative. In some embodiments, staining buffer composition is provided comprising 20-40 mg/mL monomer A, 0.5-1.5 mg/mL quenched polymer, 0.01-1% (wt/vol), nonionic surfactant and a biological buffer, optionally with a protein stabilizer and a preservative.

The staining buffer compositions according to the disclosure reduce, substantially reduce, or eliminate non-specific polymer-polymer interactions between fluorescent polymer dye conjugates, when compared to the fluorescent polymer dye conjugates in the absence of the composition. The composition according to the disclosure reduces, substantially reduces, or eliminates non-specific polymer-polymer interactions of the at least one fluorescent polymer dye conjugate, when compared to the at least one fluorescent polymer dye conjugate in the absence of the composition. Methods

The disclosure also relates to a method for detecting an analyte in a sample comprising: contacting a sample that is suspected of containing an analyte with a composition described herein. A binding partner present, e.g., in the polymer dye conjugates described herein, is capable of interacting with the analyte to form a polymer dye conjugate complex with the analyte. A light source is applied to the sample that can excite the polymer dye conjugate complex with the analyte and light emitted from the conjugated polymer complex is detected. In some embodiments, polymer dye conjugates described herein are excitable with a light having wavelength within a range of between about 340 nm and about 800 nm, about 340 nm and about 450 nm (e.g., between about 395 nm and about 415 nm). In some embodiments, emitted light is typically between about 400 nm and about 800 nm (e.g., about 400 nm and about 500 nm or about 415 nm and about 475 nm). Alternatively, excitation light can have a wavelength between about 340 nm and about 370 nm and the emitted light may be between about 390 nm and about 420 nm.

The sample in the methods of the disclosure can be, for example, blood, bone marrow, spleen cells, lymph cells, bone marrow aspirates (or any cells obtained from bone marrow), urine (lavage), serum, saliva, cerebral spinal fluid, urine, amniotic fluid, interstitial fluid, feces, mucus, or tissue (e.g., tumor samples, disaggregated tissue, disaggregated solid tumor). The sample can be a blood sample. The blood sample can be whole blood. The whole blood can be obtained from the subject using standard clinical procedures. The sample can be a subset of one or more cells of whole blood (e.g., erythrocyte, leukocyte, lymphocyte (e.g., T cells, B cells or NK cells), phagocyte, monocyte, macrophage, granulocyte, basophil, neutrophil, eosinophil, platelet, or any cell with one or more detectable markers). The whole blood sample may be a processed whole blood sample. The sample can be from a cell culture.

The subject can be a human (e.g., a patient suffering from a disease), a commercially significant mammal, including, for example, a monkey, cow, or horse. Samples can also be obtained from household pets, including, for example, a dog or cat. The subject can be a laboratory animal used as an animal model of disease or for drug screening, for example, a mouse, a rat, a rabbit, or guinea pig.

An “analyte” as used herein, refers to a substance, e.g., molecule, whose abundance/concentration is determined by some analytical procedure. For example, in the present invention, an analyte can be a protein, peptide, nucleic acid, lipid, carbohydrate or small molecule.

Assay systems utilizing a binding partner and a fluorescent label to quantify bound molecules are well known. Examples of such systems include flow cytometers, scanning cytometers, imaging cytometers, fluorescence microscopes, and confocal fluorescent microscopes.

Flow cytometry is used to detect fluorescence. A number of devices suitable for this use are available and known to those skilled in the art. Examples include BCI Navios, Gallios, Aouicts, and CytoFLEX flow cytometers.

The assay can be an immunoassay. Examples of immunoassays useful in the invention include, but are not limited to, fluoroluminescence assay (FLA), and the like. The assays can also be carried out on protein arrays.

When the binding partners are antibodies, antibody or multiple antibody sandwich assays can also be used. A sandwich assay refers to the use of successive recognition events to build up layers of various binding partners and reporting elements to signal the presence of a particular analyte. Examples of sandwich assays are disclosed in U.S. Pat. No. 4,486,530 and in the references noted therein.

### Kits

The disclosure provides kits comprising the staining buffer compositions according to the disclosure. The kit may comprise one or more containers comprising the staining buffer composition, comprising one or more non-fluorescent components of a polymer dye, and a biological buffer; and optionally one or more separate containers comprising one or more fluorescent polymer dye conjugates. The kit may include one or more of the non-fluorescent components of a first polymer dye and a nonionic surfactant in one container; and at least one fluorescent polymer dye conjugate in a separate container. The kit may comprise two or more of the non-fluorescent components of a first polymer dye in one container; and the at least one fluorescent polymer dye conjugate in a separate container. The kit may include one or more containers comprising a staining buffer according to the disclosure and a multiplicity of separate containers each comprising a different polymer dye conjugate.

The kit may include one or more components suitable for lysing cells. The one or more additional components of the kit may be provided in separate containers (e.g., separate tubes, bottles, or wells in a multi-well strip or plate).

The kit may also include one or more cell fixing reagents such as paraformaldehyde, glutaraldehyde, methanol, acetone, formalin, or any combinations or buffers thereof. Further, the kit may include a cell permeabilizing reagent, such as methanol, acetone or a detergent such as triton, NP-40, saponin, tween 20, digitonin, leucoperm, or any combinations or buffers thereof.

The kits may include instructions for using the staining buffer compositions of the disclosure, for example, in conjunction with a multi-color panel of polymer dye conjugates. The instructions may be in printed form, kit packaging, in a package insert, or a website address.

### Definitions

The abbreviations used herein have their conventional meaning within the chemical and biological arts.

As used herein, the term “ammonium” refers to a cation having the formula NHR3+ where each R group, independently, is hydrogen or a substituted or unsubstituted alkyl, aryl, aralkyl, or alkoxy group. Preferably, each of the R groups is hydrogen.

As used herein, “oligoether” is understood to mean an oligomer containing structural repeat units having an ether functionality. As used herein, an “oligomer” is understood to mean a molecule that contains one or more identifiable structural repeat units of the same or different formula.

The term “sulfonate functional group” or “sulfonate,” as used herein, refers to both the free sultanate anion (—S(═O)₂O—) and salts thereof. Therefore, the term sulfonate encompasses sulfonate salts such as sodium, lithium, potassium and ammonium sulfonate.

The term “sulfonamide” as used herein refers to a group of formula —SO₂NR— where R is hydrogen, alkyl or aryl.

The term “alkyl” as used herein refers to a straight or branched, saturated, aliphatic radical having the number of carbon atoms indicated. For example, C₁-C₆ alkyl includes, but is not limited to, methyl, ethyl, propyl, isopropyl, butyl, isobutyl, sec-butyl, tert-butyl, pentyl, isopentyl, hexyl, etc. Other alkyl groups include, but are not limited to heptyl, octyl, nonyl, decyl, etc. Alkyl can include any number of carbons, such as 1-2, 1-3, 1-4, 1-5, 1-6, 1-7, 1-8, 1-9, 1-10, 2-3, 2-4, 2-5, 2-6, 3-4, 3-5, 3-6, 4-5, 4-6 and 5-6. The alkyl group is typically monovalent, but can be divalent, such as when the alkyl group links two moieties together.

The term “co-monomer” or “co-monomer group” refers to a structural unit of a polymer that may itself be part of a repeating unit of the polymer.

The term “cycloalkyl” as used herein refers to a saturated or partially unsaturated, monocyclic, fused bicyclic or bridged polycyclic ring assembly containing from 3 to 12 ring atoms, or the number of atoms indicated monocyclic rings include, for example, cyclopropyl, cyclobutyl, cyclopentyl, cyclohexyl, and cyclooctyl. Bicyclic and polycyclic rings include, for example, norbornane, decahydronaphthalene and adamantane. For example, C₃₋₈cycloalkyl includes cyclopropyl, cyclobutyl, cyclopentyl, cyclohexyl, cyclooctyl, and norbornane.

The term “haloalkyl” as used herein refers to alkyl as defined above where some or all of the hydrogen atoms are substituted with halogen atoms. Halogen (halo) preferably represents chloro or fluoro, but may also be bromo or iodo. For example, haloalkyl includes trifluoromethyl, flouromethyl, 1,2,3,4,5-pentafluoro-phenyl, etc. The term “perfluoro” defines a compound or radical which has at least two available hydrogens substituted with fluorine. For example, perfluorophenyl refers to 1,2,3,4,5-pentafluorophenyl, perfluoromethane refers to 1,1,1-trifluoromethyl, and perfluoromethoxy refers to 1,1,1-trifluoromethoxy.

As used herein, the term “halogen” refers to fluorine, chlorine, bromine and iodine.

The term “alkoxy” as used herein refers to an alkyl group, as defined above, having an oxygen atom that connects the alkyl group to the point of attachment. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. The alkoxy groups can be further substituted with a variety of substituents described within. For example, the alkoxy groups can be substituted with halogens to form a “halo-alkoxy” group.

The term “alkene” as used herein refers to either a straight chain or branched hydrocarbon, having at least one double bond. Examples of alkene groups include, but are not limited to, vinyl, propenyl, isopropenyl, 1-butenyl, 2-butenyl, isobutenyl, butadienyl, 1-pentenyl, 2-pentenyl, isopentenyl, 1 ,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. The alkene group is typically monovalent, but can be divalent, such as when the alkenyl group links two moieties together.

The term “alkyne” as used herein refers to either a straight chain or branched hydrocarbon, having at least one triple bond. Examples of alkynyl groups include, but are not limited to, acetylenyl, propynyl, 1-butynyl, 2-butyryl, isobutynyl, sec-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl. The alkynyl group is typically monovalent, but can be divalent, such as when the alkynyl group links two moieties together.

The term “aryl” or “aromatic” as used herein refers to a monocyclic or fused bicyclic, tricyclic or greater, aromatic ring assembly containing 6 to 16 ring carbon atoms. For example, aryl may be phenyl, benzyl, naphthyl, dihydrophenanthrenyl (DHP), 9,10-dihydrophenanthrenyl, or fluorenyl. “Arylene” means a divalent radical derived from an aryl group. Aryl groups can be mono-, di- or tri-substituted by one, two or three radicals selected from alkyl, alkoxy, aryl, hydroxy, halogen, cyano, amino, amino-alkyl, trifluoromethyl, alkylenedioxy and oxy-C₂-C₃-alkylene; all of which are optionally further substituted, for instance as hereinbefore defined; or 1- or 2-naphthyl; or 1- or 2-phenanthrenyl. Alkylenedioxy is a divalent substitute attached to two adjacent carbon atoms of phenyl, e.g., methylenedioxy or ethylenedioxy. Oxy-C₂-C₃-alkylene is also a divalent substituent attached to two adjacent carbon atoms of phenyl, e.g., oxyethylene or oxypropylene. An example for oxy-C₂-C₃-alkylene-phenyl is 2,3-dihydrobenzofuran-5-yl.

Preferred as aryl is dihydrophenanthrenyl (DHP), 9,10-dihydrophenanthrenyl, naphthyl, phenyl or phenyl mono- or disubstituted by alkoxy, phenyl, halogen, alkyl or trifluoromethyl, especially phenyl or phenyl-mono- or disubstituted by alkoxy, halogen or trifluoromethyl, and in particular phenyl.

The term “aryloxy” as used herein refers to a O-aryl group, wherein aryl is as defined above. An aryloxy group can be unsubstituted or substituted with one or two suitable substituents. The term “phenoxy” refers to an aryloxy group wherein the aryl moiety is a phenyl ring. The term “heteroaryloxy” as used herein means an —O-heteroaryl group, wherein heteroaryl is as defined below. The term “(hetero)aryloxy” is use to indicate the moiety is either an aryloxy or heteroaryloxy group.

The term “AFU” refers to arbitrary fluorescence unit.

The term “AUF” refers to arbitrary units of fluorescence.

The terms “Polyethylene glycol” or “PEG” as used herein refer to the family of blocompatible water-solubilizing linear polymers based on the ethylene glycol monomer unit.

The term “heteroaryl” or “heteroaromatic” as used herein refers to a monocyclic or fused bicyclic or tricyclic heteroaromatic ring assembly containing 5 to 16 ring atoms, where from 1 to 4 of the ring atoms are a heteroatom each N, O or S. For example, heteroaryl includes pyridyl, indolyl, indazolyl, quinoxalinyl, quinolinyl, isoquinolinyl, benzothienyl, benzofuranyl, carbazolyl, furanyl, pyrrolyl, thiazolyl, benzothiazolyl, oxazolyl, isoxazolyl, triazolyl, tetrazolyl, pyrazolyl, imidazolyl, thienyl, or any other radicals substituted, especially mono- or di-substituted, by, e.g., alkyl, nitro or halogen. Pyridyl represents 2-, 3- or 4-pyridyl, advantageously 2- or 3-pyridyl. Thienyl represents 2- or 3-thienyl. Quinolinyl represents preferably 2-, 3- or 4-quinolinyl. Isoquinolinyl represents preferably 1-, 3- or 4-isoquinolinyl. Benzopyranyl, benzothiopyranyl represents preferably 3-benzopyranyl or 3-benzothlopyranyl, respectively. Thiazolyl represents preferably 2- or 4-thiazolyl, and most preferred, 4-thiazolyi. Triazolyl is preferably 1-, 2- or 5-(1,2,4-triazolyl). Tetrazolyl is preferably 5-tetrazolyl.

Preferably, heteroaryl is pyridyl, indolyl, quinolinyl, pyrrolyl, thiazolyl, isoxazolyl, triazolyl, tetrazolyl, pyrazolyl, imidazolyl, thienyl, furanyl, benzothiazolyl, benzofuranyl, isoquinolinyl, benzothienyl, oxazolyl, indazolyl, or any of the radicals substituted, especially mono- or di-substituted.

Similarly, substituents for the aryl and heteroaryl groups are varied and are selected from: -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—C(O)NR″R″′, —NH—C(NH₂)═NH, —NR′C(NH₂)═NH, —NH—C(NH₂)═NR′, —S(O)P′, —S(O)₂R′, —S(O)₂NR′R″; —N₃, —CH(Ph)₂, perfluoro(C₁-C₄)alkoxy, and perfluoro(C₁-C₄)alkyl, in a number ranging from zero to the total number of open valences on the aromatic ring system; and where R′, R″ and R′″ are independently selected from hydrogen, (C₁-C₅)alkyl and heteroalkyl, unsubstituted aryl and heteroaryl, (unsubstituted aryl)-(C₁-C₄)alkyl, and (unsubstituted aryl)oxy-(C₁-C₄)alkyl.

Two of the substituents on adjacent atoms of the aryl or heteroaryl ring may optionally be replaced with a substituent of the formula -T-C(O)—(CH₂)_{q}—U—, wherein T and U are independently —NH—, —O—, —CH₂— or a single bond, and q is an integer of from 0 to 2. Alternatively, two of the substituents on adjacent atoms of the aryl or heteroaryl ring may optionally be replaced with a substituent of the formula -A-(OH₂)ᵣ—B—, wherein A and B are independently —CH₂—, —O—, —NH—, —S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X—(CH₂)ₜ—, where s and t are independently integers of from 0 to 3, and X is —O—, —NR′—, —S—, —S(O)—, —S(O)₂—, or —S(O)₂NR′—. The substituent R′ in —NR′— and —S(O)₂NR′— is selected from hydrogen or unsubstituted (C₁-C₆)alkyl.

The term “(hetero)arylamino” as used herein refers an amine radical substituted with an aryl group (e.g., —NH-aryl). An acylamino may also be an aryl radical substituted with an amine group (e.g., -aryl-NH₂). Arylaminos may be substituted or unsubstituted.

The term “amine” as used herein refers to an alkyl groups as defined within, having one or more amino groups. The amino groups can be primary, secondary or tertiary. The alkyl amine can be further substituted with a hydroxy group. Amines useful in the present invention include, but are not limited to, ethyl amine, propyl amine, isopropyl amine, ethylene diamine and ethanolamine. The amino group can link the alkyl amine to the point of attachment with the rest of the compound, be at the omega position of the alkyl group, or link together at least two carbon atoms of the alkyl group. One of skill in the art will appreciate that other alkyl amines are useful in the present invention.

The term “carbamate” as used herein refers to the functional group having the structure —NR″CO₂R′, where R′ and R″ are independently selected from hydrogen, (C₁-C₈)alkyl and heteroalkyl, unsubstituted aryl and heteroaryl, (unsubstituted aryl)-(C₁-C₄alkyl, and (unsubstituted aryl)oxy-(C₁-C₄)alkyl. Examples of carbamates include t-Boc, Fmoc, benzyloxy-carbonyl, alloc, methyl carbamate, ethyl carbamate, 9-(2-sulfo)fluorenylmethyl carbamate, 9-(2,7-dibromo)fluorenylmethyl carbamate, Tbfmoc, Climoc, Bimoc, DBD-Tmoc, Bsmoc, Trac, Teoc, 2-phenylethyl carbamate, Adpoc, 2-chloroethyl carbamate, 1,1-dimethyl-2-haloethyl carbamate, DB-t-BOC, TCBOC, Bpoc, t-Bumeoc, Pyoc, Bnpeoc, V-(2-pivaloylamino)-1,1-dimethylethyl carbamate, NpSSPeoc.

The term “carboxylate” as used herein refers to the conjugate base of a carboxylic acid, which generally can be represented by the formula RCOO. For example, the term “magnesium carboxylate” refers to the magnesium salt of the carboxylic acid.

The term “activated ester” as used herein refers to carboxyl-activating groups employed in peptide chemistry to promote facile condensation of a carboxyl group with a free amino group of an amino acid derivative. Descriptions of these carboxyl-activating groups are found in general textbooks of peptide chemistry; for example K. D. Kopple, “Peptides and Amino Acids”, W. A. Benjamin, Inc., New York, 1966, pp. 50-51 and E. Schroder and K. Lubke, “The Peptides”; Vol. 1, Academic Press, New York, 1965, pp. 77-128.

The terms “hydrazine” and “hydrazide” refer to compounds that contain singly bonded nitrogens, one of which is a primary amine functional group.

The term “aldehyde” as used herein refers to a chemical compound that has an —CHO group.

The term “thiol” as used herein refers to a compound that contains the functional group composed of a sulfur-hydrogen bond. The general chemical structure of the thiol functional group is R—SH, where R represents an alkyl, alkene, aryl, or other carbon-containing group of atoms.

The term “silyl” as used herein refers to Si(R^{z})₃ wherein each R^{z} independently is alkyl aryl or other carbon-containing group of atoms.

The term “diazonium salt” as used herein refers to a group of organic compounds with a structure of R—N₂⁺X⁻, wherein R can be any organic residue (e.g., alkyl or aryl) and X is an inorganic or organic anion (e.g., halogen).

The term “triflate” also referred to as trifluoromethanesulfonate, is a group with the formula CF₃SO₃.

The term “boronic acid” as used herein refers to a structure —B(OH)₂. It is recognized by those skilled in the art that a boronic acid may be present as a boronate ester at various stages in the synthesis of the quenchers. Boronic acid is meant to include such esters. The term “boronic ester” or “boronate ester” as used herein refers to a chemical compound containing a —B(Z¹)(Z²) moiety, wherein Z¹ and Z² together form a moiety where the atom attached to boron in each case is an oxygen atom. The boronic ester moiety can be a 5-membered ring. The boronic ester moiety can be a 6-membered ring. The boronic ester moiety can be a mixture of a 5-membered ring and a 6-membered ring.

The term “DABCYL” is an acronym for a 4-(dimethylaminoazo)benzene-4-carboxylic acid. DABCYL may be employed as a quenching moiety. DABCYL has an absorption maximum about 474 nm.

The term “DABSYL” refers to 4-(dimethylaminoazo)benzene-4″-sulfonyl chloride. DABSYL may be employed as a quenching moiety.

The term “Black Hole Quencher 1” (BHQ-1) refers to a quenching moiety having an absorption max about 534 nm.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

The term “CD” refers to Cluster of differentiation.

The term “Compensation” in flow cytometry is a mathematical process of correcting for fluorescence spillover (spectral overlap of multiparameter flow cytometric data). For example, compensation may be performed by removing the signal of any given fluorochrome from all detectors except the one devoted to measuring that dye. Since fluorochromes may have wide-ranging spectrum, they can overlap, causing the undesirable confusion during data analysis.

The term “MdFI” or “MDFI” refers to Median fluorescent intensity.

The term MFI=Mean Fluorescence Intensity.

The term “% recruitment” refers to number of gated cells of relevant population.

The term “Multi-Color dye conjugate panel” or “Multi-Color antibody panel” refers to a cocktail comprising a plurality of different fluorescent dye conjugates (e.g., CD4-FITC, CD8-PE, CD20-APC, CD3-PC5.5, CD16-FITC, CD25-PE, CD3-ECD, CD38-PC5.5, CD27-PC7, CD10-APC, CD14-APCA700, CD45-AA750, CD8-KRO, CD56-SNv428, CD20-SNv605, CD4-SNv786, etc.) that may be used directly to stain blood and analyze it in a flow cytometer.

The term “Multiplexing” herein refers to an assay or other analytical method in which multiple analytes can be assayed simultaneously.

Water solubilizing moieties may be included in the polymer dye to provide for increased water-solubility. While the increase in solubility may vary, in some instances the increase compared to the polymer dye without water-solubilizing moieties may be at least 2 fold or more, e.g., 5 fold, 10 fold, 25 fold, 50 fold, 100 fold or more.

The term “water solubilizing moiety” refers to a group that is well solvated in aqueous environments e.g., under physiological conditions, and that imparts improved water solubility upon the molecules to which it is attached. The water solubilizing moiety may be any appropriate hydrophilic group that is well solvated in aqueous environments. In some cases, the hydrophilic water solubilizing group is charged, e.g., positively or negatively charged. In certain cases, the hydrophilic water solubilizing group is a neutral hydrophilic group. In some embodiments, the water solubilizing moiety is a hydrophilic polymer, e.g., a polyethylene glycol, a cellulose, a chitosan, or a derivative thereof. Water solubilizing moieties may include, but are not limited to, carboxylate, phosphonate, phosphate, sulfonate, sulfate, sulfinate, sulfonium, ester, polyethylene glycols (PEG) and modified PEGs, hydroxyl, amine, ammonium, guanidinium, pyridinium, polyamine and sulfonium, polyalcohols, straight chain or cyclic saccharides, primary, secondary, tertiary, or quaternary amines and polyamines, phosphonate groups, phosphinate groups, ascorbate groups, glycols. In some embodiments, the water solubilizing moiety is a PEG.

The term “PEG” refers to polyethylene glycol, or poly(ethylene glycol), the family of biocompatible water-solubilizing linear polymers based on the ethylene glycol monomer unit described by the formula —(CH₂—CH₂—O—)ₙ— or a derivative thereof. The water-solubilizing moiety may be capable of imparting solubility in water of at least 10 mg/mL. A PEG moiety may be employed as a water-solubilizing moiety. In some embodiments, “n” is 1000 or less, 500 or less, 200 or less, 100 or less, 50 or less, 40 or less, 30 or less, 20 or less, 15 or less, such as 3 to 15, or 10 to 15. It is understood that the PEG polymeric group may be of any convenient length and may include a variety of terminal groups and/or further substituent groups, including but not limited to, alkyl, aryl, hydroxyl, amino, acyl, carboxylic acid, carboxylate ester, acyloxy, and amido terminal and/or substituent groups. The number after “PEG” refers to the average molecular weight, where Mw refers to weight average molecular weight, and Mn refers to number average molecular weight.

The term “non-fluorescent component of a polymer dye” refers to a monomeric unit of a polymer dye, a photo-bleached polymer dye, a polymer dye comprising a quenching moiety, or a non-fluorescent polymer dye, wherein the non-fluorescent component of a polymer dye exhibits little to no ability to re-emit light upon light excitation. The non-fluorescent component of a polymer dye may exhibit a quantum yield of no more than about 0.1, 0.06, or 0.056. The non-fluorescent component of a polymer dye may emit ≤50 AFU at 450 nm (Slits ex/em are 6 nm/4 nm; 1 cm cuvette) for 10 micrograms/mL. The non-fluorescent component of a polymer dye may have less than about 50 AFU when excited by a 405 nm laser. The non-fluorescent component of a polymer dye may exhibit >95% quenching of original maximum emission intensity, >98% quenching of original maximum emission intensity, up to 100% quenching compared to parent fluorescent polymer when excited at 405 nm.

The term “non-fluorescent polymer dye” refers to a polymer dye according to formula (I) that exhibits a quantum yield of no more than about 0.1, 0.06, or 0.056 without photo-bleaching and that does not comprise a quenching moiety. The non-fluorescent polymer dye may have less than about 50 AFU when excited by a 405 rim laser. The non-fluorescent polymer dye may exhibit >95% quenching of original maximum emission intensity, >98% quenching of original maximum emission intensity, up to 100% quenching compared to parent fluorescent polymer when excited at 405 nm.

The term “non-specific binding” as used herein refers generally to any binding which is not caused by specific binding, and more specifically to the binding of polymer dye conjugates by means other than specific binding of the binding partner to the target analyte. Non-specific binding may result from several factors, including hydrophobicity of the polymers, immune complexing agents, charged proteins, and antibody-interfering proteins which may be present in the staining buffer or biological sample. One type of non-specific binding is the polymer-polymer interactions that may occur between one or more, or two or more fluorescent polymer dye conjugates. Non-specific binding in a test staining buffer composition may be assessed by, for example, comparing FCA dot plots of a mixture of multi-color fluorescent polymer dye conjugates in a biological sample to FCA dot plots of the individual single color fluorescent polymer dye conjugates of the mixture in the same sample, for example, according to the methods provided herein. For example, If the solution is efficient in preventing non-specific binding polymer-polymer interactions, the respective cell populations will appear well compensated similarly to the staining obtained with the single color conjugates used individually. On the contrary, if the solution is poorly efficient, the populations won't be aligned and will look tilted.

An alternative method for measuring the efficiency of the staining buffer compositions for reducing non-specific binding such as polymer-polymer interactions according to the disclosure uses the MFI of the negative and positive populations of the conjugates when they are used individually versus mixed.

The term “photo-bleached dye” refers to a dye originally comprising a fluorophore that has undergone high-intensity illumination such that it can no longer fluoresce. In some embodiments, the photo-bleached polymer exhibits a quantum yield (QY) no more than 0.1, or no more than 0.06, or no more than 0.056, no more than 0.05, no more than 0.02, or no more than 0.015ϕ. In some embodiments, the photo-bleached polymer exhibits less than about 50 arbitrary units of fluorescence (AFU) when excited with a 405 nm laser. The photo-bleached dye may exhibit >95% quenching of original maximum emission intensity, >98% quenching of original maximum emission intensity, up to 100% quenching compared to parent fluorescent polymer when excited at 405 nm.

A fluorophore can undergo the fluorescence process repeatedly. This means that the fluorophore molecule can theoretically generate a signal multiple times. In reality, the fluorophore's structural instability during its excited lifetime may make it susceptible to degradation. High-intensity illumination can cause the fluorophore to change its structure so that it can no longer fluoresce and this is called photo-bleaching.

The term “quantum yield” (QY) (ϕ) or “fluorescence quantum yield” refers to the ratio of the number of photons emitted to the number of photons absorbed. The quantum yield is independent of instrument settings and describes how efficiently a fluorophore converts the excitation energy into fluorescence. Experimentally the relative fluorescence quantum yields can be determined by measuring fluorescence of a fluorophore of known quantum yield with same experimental parameters (excitation wavelength, slit widths, photomultiplier voltage, etc.) as the test dye. The quantum yield may be determined by any method known in the art. For example, the QY may be determined per manufacturer's instructions in a fluorescence spectrofluorometer or fluorescence spectrometer at a selected excitation wavelength. For example, Quantum yield (QY) may determined on a Shimadzu Rf-6000 Fluorescence Spectrofluorometer by measuring emission intensity at 428 nm from a diluted PBS solution of staining buffer with absorbance at 405 nm=0.05 (excitation at 405 nm, ex slit 1.5, em slit 3.0, 1 cm quartz cuvette). The quantum yield may be calculated, for example, by comparing the intensity measured from the sample and the intensity measured from a NHS Pacific Blue (QY=0.78 in PBS 1×x) solution under the same experimental conditions. In some embodiments, the QY may be determined, for example, Lawson-Wood et al., Application Note-Fluorescence Spectroscopy, Determination of relative fluorescence quantum yield using the FL5600 fluorescence spectrometer, 2018, PerkinElmer, Inc. The selected excitation wavelength may be, for example 405 nm. In some embodiments, the QY of a quenched polymer or photo-bleached polymer may be compared to parent fluorescent polymer absent photo-bleaching and without comprising a quenching moiety.

The term “fluorescent dye” refers to a dye comprising a light excitable fluorophore that can re-emit light upon light excitation. The term “fluorescent dye” encompasses both fluorescent polymeric dyes and fluorescent non-polymeric dyes, including fluorescent monomeric and other traditional fluorescent dyes. The fluorescent polymer dye may be any appropriate fluorescent polymer dye, for example, comprising a structure according to the disclosure. Fluorescent polymer dyes are also commercially available. For example, SuperNova™ (“SN”) v428 (Beckman Coulter, Inc.) is a fluorescent polymer dye optimally excited by the violet laser (405 nm) with an excitation maximum of 414 nm, an emission peak of 428 nm, and can be detected using a 450/50 bandpass filter or equivalent. SN v605 and SN v786 are tandem polymer dyes, derived from the core SN v428 polymer dye. Both share the same absorbance characteristics, with maximum excitation at 414 nm. With emission peaks for SN v605 and SN v786 at 605 nm and 786 nm respectively, they are optimally detected using the 610/2 and 780/60 nm bandpass filters of the flow cytometer.

The term “fluorophore” refers to a fluorescent chemical compound that can re-emit light upon light excitation. Fluorophores may typically contain several combined aromatic groups, or planar and cyclic molecules with several p pi bonds.

The terms, “patient”, “subject” or “subjects” include but are not limited to humans, the term may also encompass other mammals, or domestic or exotic animals, for example, dogs, cats, ferrets, rabbits, pigs, horses, cattle, birds, or reptiles.

Unless otherwise specified, the term phrase “room temperature” refers to 18 to 27° C.

Unless otherwise specified, the term “percent”, or “%” refers to weight percent.

The phrases “ready to use reagent”, “ready to use reagent composition”, “working concentration reagent”, and “working concentration reagent composition” refer to a staining buffer composition produced at about 1X working concentration appropriate for use, for example, in a mixture of polymer dye conjugates for staining a biological sample for flow cytometry analysis (FCA).

The phrase “concentrated staining buffer” or “concentrated staining buffer composition”, refers to staining buffer composition produced at, for example, about a 10-fold concentration factor (10×) for dilution, for example, with a diluent such as a biological buffer or water, to provide a working concentration staining buffer composition useful for decreasing non-specific polymer interactions in a multi-color panel when staining a biological sample for flow cytometry analysis. The concentrated staining buffer composition may be manufactured and remain stable in a concentration from 1-fold (1×) to at least 10-fold (10×), or at least 1-fold, 2-fold, 3-fold, 4-fold, 5-fold, 6-fold, 7-fold, 8-fold, 9-fold, or 10-fold more concentrated than the working concentration staining buffer composition.

In some embodiments, the working concentration staining buffer composition is stable for at least 3 months, at least 6 months, at least 9 months, at least 12 months, at least 18 months, or at least 24 months or more from the date of manufacture when stored in unopened original container at a temperature within a range of from 2 to 8° C., with excursions to 15 to 37° C., or ambient temperature 19 to 27° C. In some embodiments, the concentrated staining buffer composition is stable for at least 3 months, 6 months, 9 months, 12 months, 18 months, 24 months, 30 months, or 36 months or more from the date of manufacture when stored in unopened original container at a temperature within a range of from 2 to 8° C., with excursions to 15 to 37° C., or at ambient temperature 19 to 27° C.

The acronym “SN” refers to SuperNova™.

The acronym “SSC” refers to side scatter.

The term “WBC” refers to white blood cells.

The term “about,” when referring to a measurable value such as an amount of a compound, dose, time, temperature, and the like, is meant to encompass variations of 10%, 5%, 1%, 0.5%, or even 0.1% of the specified amount, and at least industry-standard variation in the test method for measuring the value.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading can occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

As used herein, “reducing” or “eliminating” of non-specific binding of the polymer dye conjugate can refer to when the “negatives” (e.g., negative granulocyte, monocyte, and lymphocyte populations) mean fluorescence intensity (MFI), in % relative to when no non-fluorescent component of a first polymer dye is used, is decreased by at least about 50% (e.g., by at least about 60%, at least about 70%, at least about 80%, at least about 90%, at least about 95%, at least 99% or more; from about 50% to about 95%, about 50% to about 75%, about 60% to about 80% or about 65% to about 90%). In other words, the % reduction of at least one of monocyte, granulocyte, and lymphocyte background staining, in % relative to when no surfactant is used, is decreased by at least about 50% (e.g., by at least about 60%, at least about 70%, at least about 80%, at least about 90%, at least about 95%, at least 99% or more; from about 50% to about 95%, about 50% to about 75%, about 60% to about 80% or about 65% to about 90%).

In the methods described herein, the steps can be carried out in any order without departing from the principles of the invention, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

Each embodiment described above is envisaged to be applicable in each combination with other embodiments described herein. For example, embodiments corresponding to Formula (I) are equally envisaged as being applicable to Formulae (VIII)-(XIV), (XVIII), (XIX), (XX). As another example, embodiments corresponding to any of Formulae (VIII)-(XIV), (XVIII), (XIX), (XX) are equally envisaged as being applicable to Formula (I).

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The term “substantially no” or “substantially free of” as used herein refers to less than about 1%, 0.5%, 0.1%, 0.05%, 0.001%, or at less than about 0.0005% or less, about 0%, below quantitation limits, below detectable limits, or 0%.

Those skilled in the art will appreciate that many modifications to the embodiments described herein are possible without departing from the spirit and scope of the present disclosure. Thus, the description is not intended and should not be construed to be limited to the examples given but should be granted the full breadth of protection afforded by the appended claims and equivalents thereto. In addition, it is possible to use some of the features of the present disclosure without the corresponding use of other features. Accordingly, the foregoing description of or illustrative embodiments is provided for the purpose of illustrating the principles of the present disclosure and not in limitation thereof and can include modification thereto and permutations thereof.

### EXAMPLES

The present invention can be better understood by reference to the following examples which are offered by way of illustration. The present invention is not limited to the examples given herein.

### Example 1: Preparation of DHP Polymer Dyes

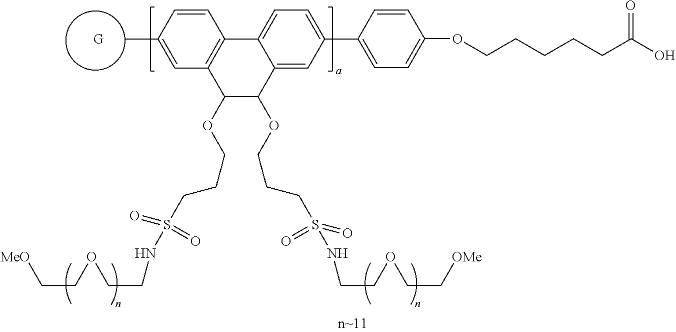

Method 1: In a round bottom flask both dibromo DHP and diboronic DHP monomers (1:1) were taken in (DMF-water) mixture and purged with nitrogen for 10 minutes. Under nitrogen about 20 equivalent of CsF and 10% of Pd(OAc)₂ were mixed and heated at 80 deg Celsius. Polymerization was monitored using UV-Vis spectroscopy and SEC chromatography. Later to the reaction mixture, a capping agent (selected from G1) containing appropriate functional group was added and 3 hours later the second capping agent (selected from G2) added. After the reaction the crude reaction mixture was evaporated off and passed through a gel filtration column to remove small organic molecules and low MW oligomers. Later the crude polymer passed through a Tangential flow filtration system equipped with a 100K MWCO membrane. It is washed using 20% ethanol until the absorption of the filtrate diminishes.

Method 2: Alternatively, the polymerization can be done by self-polymerizing a bromo-boronic ester of DHP molecule. In a round bottom flask DHP bromoboronic ester was taken in (DMF-water) mixture and purged with nitrogen for 10 minutes. Under nitrogen about 10 equivalent of CsF and 5% of Pd(OAc)₂ were mixed and heated at 80 deg Celsius. Polymerization was monitored using UV-Vis spectroscopy and SEC chromatography. Later to the reaction mixture, a capping agent (selected from G1) containing appropriate functional group was added and 3 hours later the second capping agent (selected from G2) added. After the reaction the crude reaction mixture was evaporated off and passed through a gel filtration column to remove small organic molecules and low MW oligomers. Later the crude polymer passed through a Tangential flow filtration system equipped with a 100K MWCO membrane. It is washed using 20% ethanol until the absorption of the filtrate diminishes.

Method 3: In a round bottom flask both the dibromo dihydrophenanthrene and diboronic dihydrophanenthrene monomers (1:1) were taken and dissolved in THF-water (4:1) mixture containing 10 equivalent of K₂CO₃ and 3% Pd(PPh₃)₄. The reaction mixture was put on a Schlenk line and was degassed with three freeze-pump-thaw cycles and then heated to 80 deg C. under nitrogen with vigorous stirring for 18 hours. Later to the reaction mixture, a capping agent (selected from G1) containing appropriate functional group was added via a cannula under excess nitrogen pressure and 3 hours later the second capping agent (selected from G2) added. After the reaction the crude reaction mixture was evaporated off and passed through a gel filtration column to remove small organic molecules and low MW oligomers. Later the crude polymer passed through a Tangential flow filtration system equipped with a 100K MWCO membrane. It is washed using 20% ethanol until the absorption of the filtrate diminishes.

Method 4: Alternatively the polymerization can be done by self-polymerizing a bromo-boronic ester of dihydrophenanthrene molecule. In a round bottom flask dihydrophenanthrene bromoboronic ester was taken and dissolved in THF-water (4:1) mixture containing 10 equivalent of K₂CO₃ and 3% Pd(PPh₃)₄. The reaction mixture was put on a Schlenk line and was degassed with three freeze-pump-thaw cycles and then heated to 80 deg C. under nitrogen with vigorous stirring for 18 hours. Later to the reaction mixture, a capping agent (selected from G1) containing appropriate functional group was added via a cannula under excess nitrogen pressure and 3 hours later the second capping agent (selected from G2) added. After the reaction the crude reaction mixture was evaporated off and passed through a gel filtration column to remove small organic molecules and low MW oligomers. Later the crude polymer passed through a Tangential flow filtration system equipped with a 100K MWCO membrane. It is washed using 20% ethanol until the absorption of the filtrate diminishes.

### Example 2: Preparation of Fluorene-DHP Copolymer Dyes

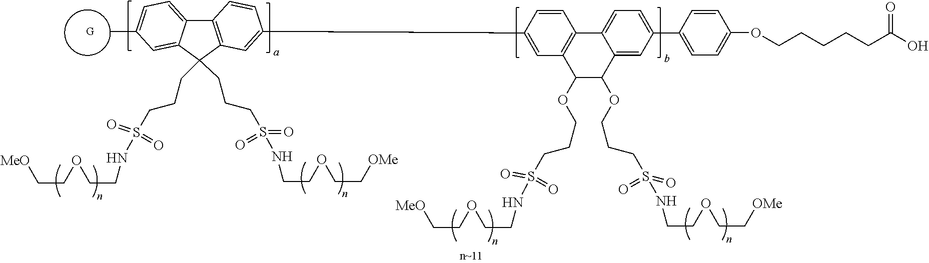

Method 1: In a round bottom flask both dibromo DHP and diboronic fluorene monomers (1:1) were taken in (DMF-water) mixture and purged with nitrogen for 10 minutes. Under nitrogen about 20 equivalent of CsF and 10% of Pd(OAc)2 were mixed and heated at 80 deg Celsius. Polymerization was monitored using UV-Vis spectroscopy and SEC chromatography. Later to the reaction mixture, a capping agent (selected from G1) containing appropriate functional group was added and 3 hours later the second capping agent (selected from G2) added. After the reaction the crude reaction mixture was evaporated off and passed through a gel filtration column to remove small organic molecules and low MW oligomers. Later the crude polymer passed through a Tangential flow filtration system equipped with a 100K MWCO membrane. It is washed using 20% ethanol until the absorption of the filtrate diminishes.

Method 2: In a round bottom flask both the dibromo fluorene and diboronic DHP monomers (1:1) were taken in (DMF-water) mixture and purged with nitrogen for 10 minutes. Under nitrogen about 20 equivalent of CsF and 10% of Pd(OAc)2 were mixed and heated at 80 deg celcius. Polymerization was monitored using UV-Vis spectroscopy and SEC chromatography. Later to the reaction mixture, a capping agent (selected from G1) containing appropriate functional group was added and 3 hours later the second capping agent (selected from G2) added. After the reaction the crude reaction mixture was evaporated off and passed through a gel filtration column to remove small organic molecules and low MW oligomers. Later the crude polymer passed through a Tangential flow filtration system equipped with a 100K MWCO membrane. It is washed using 20% ethanol until the absorption of the filtrate diminishes.

Method 3: In a round bottom flask both the dibromo dihydrophenanthrene and diboronic fluorene monomers (1:1) were taken and dissolved in THF-water (4:1) mixture containing 10 equivalent of K₂CO₃ and 3% Pd(PPh₃)₄. The reaction mixture was put on a Schlenk line and was degassed with three freeze-pump-thaw cycles and then heated to 80 deg C. under nitrogen with vigorous stirring for 18 hours. Later to the reaction mixture, a capping agent (selected from G1) containing appropriate functional group was added via a cannula under excess nitrogen pressure and 3 hours later the second capping agent (selected from G2) added. After the reaction the crude reaction mixture was evaporated off and passed through a gel filtration column to remove small organic molecules and low MW oligomers. Later the crude polymer passed through a Tangential flow filtration system equipped with a 100K MWCO membrane. It is washed using 20% ethanol until the absorption of the filtrate diminishes.

Method 4: In a round bottom flask dibromo fluorene and diboronic dihydrophenanthrene monomers (1:1) were taken and dissolved in THF-water (4:1) mixture containing 10 equivalent of K₂CO₃ and 3% Pd(PPh₃)₄. The reaction mixture was put on a Schlenk line and was degassed with three freeze-pump-thaw cycles and then heated to 80 deg C. under nitrogen with vigorous stirring for 18 hours. Later to the reaction mixture, a capping agent (selected from G1) containing appropriate functional group was added via a cannula under excess nitrogen pressure and 3 hours later the second capping agent (selected from G2) added. After the reaction the crude reaction mixture was evaporated off and passed through a gel filtration column to remove small organic molecules and low MW oligomers. Later the crude polymer passed through a Tangential flow filtration system equipped with a 100K MWCO membrane. It is washed using 20% ethanol until the absorption of the filtrate diminishes.

### Example 3: Comparison of Fluorescence Emission Spectra

Comparison of fluorescence emission spectra of fluorene (Fl—Fl), dihydrophenanthrene (DHP-DHP) and fluorene-DHP (DHP-Fl) polymers were undertaken. DHP containing polymers show a marked difference in their fluorescence maxima which is at 426-428 nm, whereas the fluorene based polymers show a maxima of 421 nm.

### Example 4: Comparison of Absorption Spectra

The absorption spectra of both fluorene (Fl—Fl) polymer and dihydrophenanthrene (DHP-DHP) polymer were measured. The graph shows absorption of the DHP-DHP polymer (black curve) at 390 and 410 nm, whereas the Fl—Fl (grey curve) polymer shows the maxima around 400 nm. Samples were measured under different concentration.

### Example 5: CD4 Signal to Noise Ratio

The flow cytometric analysis of lysed whole blood stained with the new polymers-labeled anti-human CD4 and Pacific Blue-labeled CD4 was undertaken. The positive signal intensity of polymer dyes were nearly 5 times higher than Pacific Blue.

### Example 6: Procedure for Surface Staining With Concomitant Fixation Buffer in Sample Preparation for Flow Cytometry

In this procedure, a staining buffer according to the disclosure is added into the test tube before addition of dye conjugates in order to avoid any possible non-specific interactions that may occur between the dye conjugates over time. Fixation is a stage which enables leucocytic preparations to be stored for several hours without deterioration, after staining with a fluorescent antibody. Lysing solution may be used for lysis of red blood cells in the preparation of biological samples for flow cytometry.
1. Extemporaneously prepare the “Fix-and-Lyse” mixture by adding 25 μL of undiluted IOTest 3 10× Fixative Solution (A07800, Beckman Coulter, Inc.) to 1 mL of VersaLyse™ lysing solution (AO9777, Beckman Coulter, Inc.). Prepare a sufficient volume of the “Fix-and Lyse” mixture depending on the number of biological test samples to be lysed (1 mL of mixture per tube).
2. In each test tube, add 10 μL of the staining buffer according to the disclosure. The staining buffer is not required for test tube not containing a mix of polymer dye conjugates.
3. Add the appropriate volumes of dye conjugates. Vortex the tubes gently.
4. Add 100 μL of the test sample to each tube. Vortex the tubes gently.
5. Incubate for 15 to 20 minutes at room temperature (18-25° C.), protected from light. Then perform lysis of the red cells:
6. Add 1 ml of the “Fix-and-Lyse” mixture prepared extemporaneously and vortex immediately for one second.
7. Incubate for 10 minutes at room temperature, protected from light.
8. Centrifuge for 5 minutes at 150×g at room temperature.
9. Remove the supernatant by aspiration.
10. Resuspend the cell pellet using 3 mL of PBS.
11. Centrifuge for 5 minutes at 150×g at room temperature.
12. Remove the supernatant by aspiration.
13. Resuspend the cell pellet using 0.5 mL of PBS plus 0.1% Formaldehyde (A 0.1% formaldehyde PBS can be obtained by diluting 12.5 μL of the IOTest 3 Fixative Solution (see Catalog for PN) at its 10× concentration in 1 mL of PBS).

These preparations may be kept 24 hours between 2 and 8° C. and protected from light before analysis by flow cytometry.

### Example 7: Procedure for Photo-Bleaching Polymer Dyes

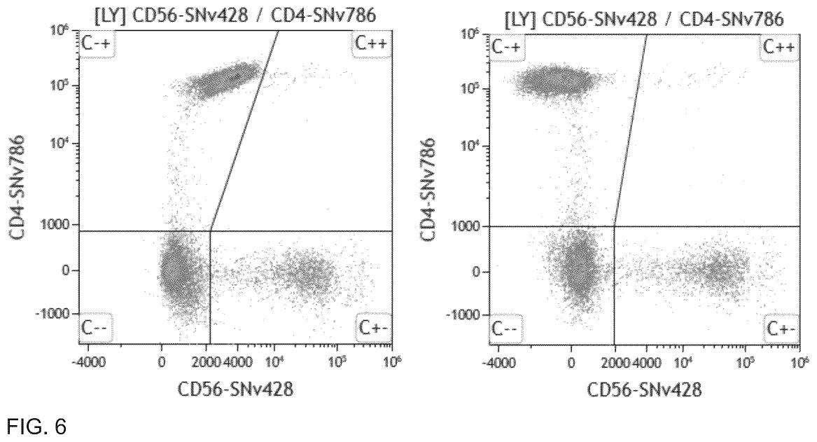

A photo-bleached polymer dye was prepared as follows. Briefly, the process includes thawing violet polymer dye 428, diluting the polymer dye at 1 mg/mL in PBA/PF-68 0.02%, placing the diluted dye into a Roux glass flask, and placing the flask into a UV Chamber (Bio-Link-BLX).—The diluted polymer dye is exposed to UV light until reaching a fluorescence value inferior or qual to 50 AFU. The residual fluorescence of the photo-bleached dye is measured by Fluorimetry (Fluorimeter LS50B, Perkin Elmer). The photo-bleached dye emits ≤50 AFU at 450 nm (Slits ex/em are 6 nm/4 nm; 1 cm cuvette) for 10 micrograms/mL to pass fluorimetry criteria. When used in a composition according to the disclosure in FCA of a blood sample, it was found that the polymer dye 428 needed to be effectively photo-bleached to avoid the appearance of non-specific staining. The remaining fluorescence of the photo-bleached dye was found to have a direct impact on the spillover of the conjugates hence on the flow cytometric results. FIG. 6 shows two FCA dot plots of a stained and lysed sample treated with polymer dye conjugates CD56-SNv428/CD4-SN786. The left panel shows the effect of inefficiently photo-bleached polymer dye, exhibiting undesirable spillover of the conjugates (arrow). The right panel shows a flow cytometry two-dimensional dot plot of two polymer dye conjugates CD56-SNv428/CD4-SN786 in which the sample is prepared with a composition according to the disclosure comprising effectively photo-bleached polymer dye 428 exhibiting no more than QY 0.056 and <47 AFU at 10 ug/mL when excited with 405 nm laser, AFU slits ex/em 6 nm/4 nm, Fluorimeter LS50B Perkin Elmer). Spillover between conjugates was substantially reduced.

### Example 8: Staining Buffer Composition Comprising Monomeric Component of a Polymer Dye and a Photo-Bleached Polymer Dye

Initial efforts to design a staining buffer composition involved testing of individual candidate components in PBS biological buffer with PF-68 using a mix of two violet polymer dye conjugates and obtaining FCA dot plots to evaluate performance. Exemplary monomer A (100-800 ug/test), maleimide violet polymer dye 428 (m428) (3.1-100 ug/test), carboxylic violet polymer dye 428 (3.1-100 ug/test), amino polymer dye 428 (3.1-100 ug/test), photo-bleached maleimide 428 (m428) dye (with and without cysteamine HCl), photo-bleached carboxylic 428 (c428) dye, photo-bleached amino polymer dye 428 (a428), PEG, or Empigen zwitterionic surfactant were evaluated individually. Exemplary monomer A and photo-bleached dyes were individually found to be efficient to reduce non-specific interactions between dye conjugates.

Based on results with individual components, a test staining buffer composition was developed using an exemplary Monomer A and photo-bleached violet dye 428 according to example 7.

PF-68 detergent was added extemporaneously into PBS/BSA/NaN 3 to reach a final concentration of 0.02%. This mix was stored at room temperature until its use to formulate the buffer. The composition was formulated by mixing the components to obtain the composition shown in Table 1.

TABLE 1
Staining Buffer Composition A

| | Final Working | | Concentrated | | |
|---|---|---|---|---|---|
| | Concentration | | Composition | | Amount |
| Component | (1X) | | (10X) | | per test |
| Exemplary | 20-40 | mg/mL | 200-400 | mg/mL | 200-400 |
| Monomer A | | | | | ug/test |
| photo-bleached | 0.2-0.8 | mg/mL | 2-8 | mg/mL | 2-8 |
| dye 428 | | | | | ug/test |
| PF-68 | 0.01-0.2% | (wt/vol) | 0.1-2% | (wt/vol) | N/A |

| | | | |
|---|---|---|---|
| PBS/BSA/NaN₃ | 1X/2 mg/mL/0.1% | N/A | N/A |

### Example 9: Performance of Test Staining Buffer Composition Comprising a Monomeric Component of a Polymer Dye and a Photo-Bleached Polymer Dye

Polymer dye antibody conjugates are antibodies conjugated to polymeric dyes which may non-specifically interact when they are mixed together. A staining buffer composition was designed in order to reduce, substantially reduce or eliminate non-specific polymer dye conjugate interactions in order allow customers to perform multi-color experiments using more than one polymer dye conjugate in their panels.

In the present example, unlike other staining buffer manufacturers, the quenching of the dye fluorescence is performed by exposing the dye to UV light according to the disclosure. Briefly, the photo-bleaching was performed by exposition of the dye to UV light (365 nm).

A photo-bleached violet dye 428 was prepared by thawing maleimide violet dye 428, diluting it at 1 mg/mL in PBS/PF-68 0.02%, placing diluted dye into a Roux glass flask, placing the flask into the UV chamber (Bio-Link-BLX) and subjecting to 3 cycles of 10 hrs of UV exposure 365 nm. The photo-bleached dye exhibited Quantum Yield (QY) of no more than 0.056. The residual fluorescence of the photo-bleached dye was also measured by Fluorimetry to 47 AFU when excited with a 405 nm laser (at 10 ug/mL, AFU slits ex/em 6 nm/4 nm, Fluorimeter LS50B Perkin Elmer).

Test staining buffer compositions were prepared comprising the photo-bleached violet dye 428, exemplary Monomer A (a subunit of dye 428), PF-68 0.02%, 1×PBS/BSA 2mg/mL/NaN₃ 0.1%.

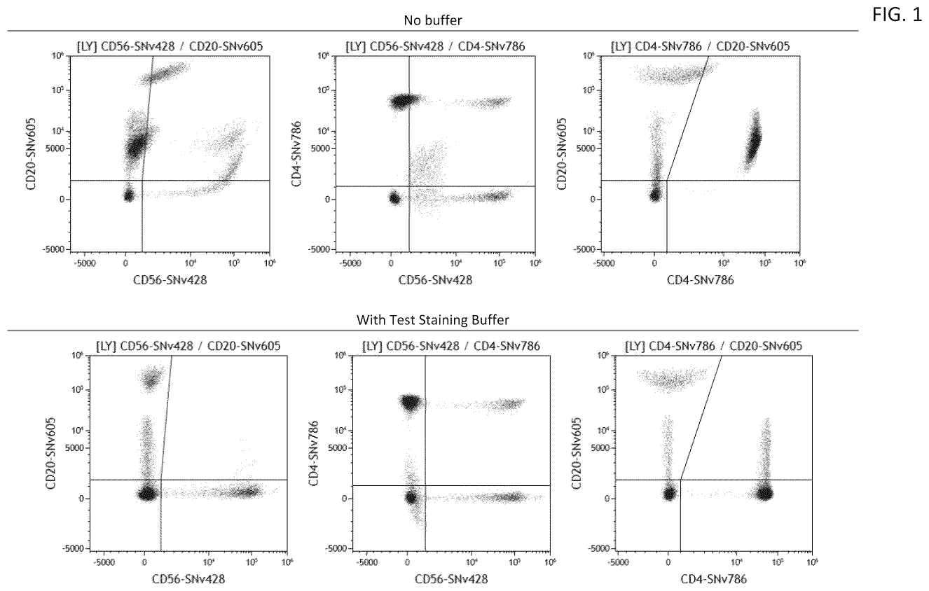

A FCA dot plot comparison of stained and lysed whole blood sample using a mixture of three SuperNova (SNv) dye antibody conjugates CD56-SNv428, CD20-SNv605, and CD4-SNv786 with or without pre-addition of Test Staining Buffer is shown in FIG. 1. Gating is on lymphocytes (LY). Acquisition was with a CytoFLEX LX flow cytometer, using CytExpert acquisition software. Analysis is with Kaluza analysis software. As shown in FIG. 1, upper three panels, abnormal staining can occur in absence of the test staining buffer and data can appear under-compensated. In the presence of test staining buffer, as shown in lower three panels, substantially reduced spillover between conjugates was demonstrated.

The efficiency of the test staining buffer may be evaluated by FCA analysis dividing the median of the spillover for the positive population with the median on the same axis of the negative population. FIG. 1, upper panels, show FCA dot blots of mix without staining buffer; showing spillover indicative of non-specific interactions between polymer dyes conjugates. In contrast, FIG. 1, lower three panels, using test staining buffer shows FCA dot plot exhibiting efficiency of the buffer to reduce non-specific polymer dye conjugate interactions.

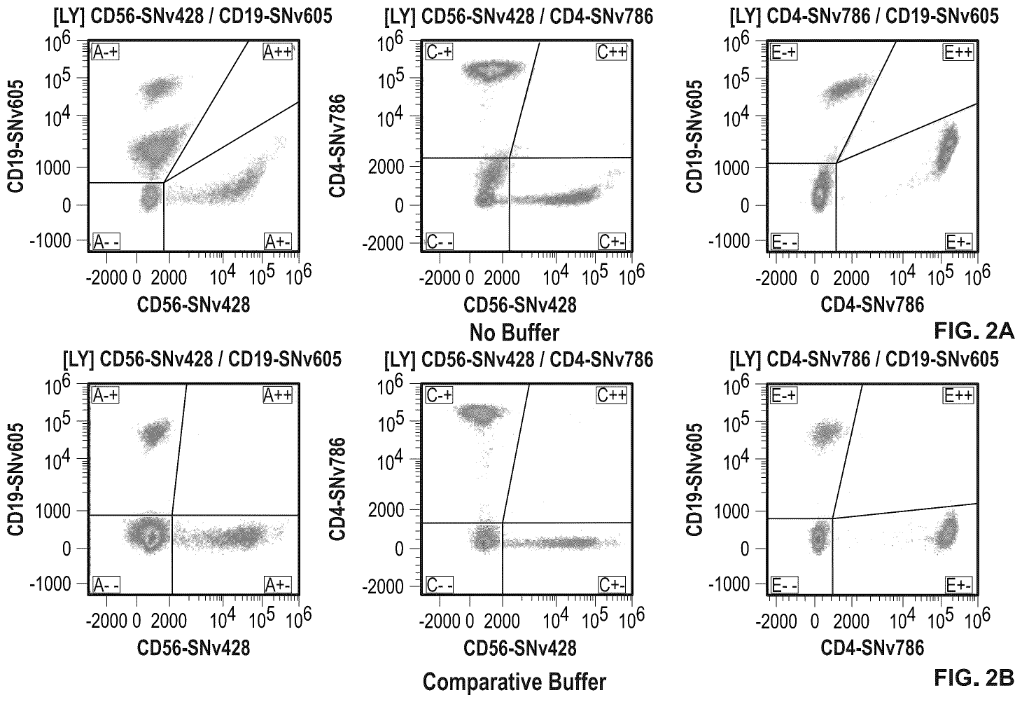

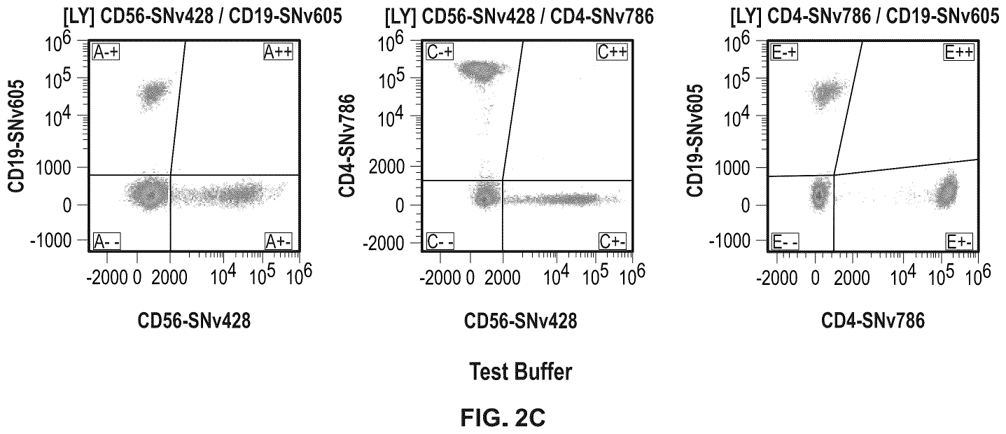

In another experiment, the test staining buffer was prepared by mixing exemplary Monomer A and Photo-Bleached dye 428 in a solution of PBS/BSA/NaN3/PF-68, as described herein (100 tests/vial; 10 μL/test). The polymer dye conjugates were added on top of the test staining buffer before adding the whole blood biological sample. A multi-color panel comprising a mixture of three SuperNova polymer dye conjugates was employed including CD56-SNv428+CD19-SNv605+CD4-SNv786. FIG. 2A shows FCA dot plots of stained and lysed whole blood sample using the multi-color panel without staining buffer. FIG. 2B shows FCA dot plots of stained and lysed whole blood sample and the multi-color panel with comparative BD Horizon™ Brilliant stain buffer (BD Biosciences). FIG. 2C shows FCA dot plots of whole blood sample and multi-color panel with Test staining buffer. The comparative and test staining buffer compositions exhibited reduced spillover compared to the multi-color panel without buffer. The test staining buffer composition exhibited somewhat reduced spillover when compared to prior art comparative buffer, indicative of reduced non-specific polymer-polymer interactions.

### Example 10: Quenched Polymers

Polymer dyes comprising various quenching moieties were prepared and investigated for possible use in staining buffers to prevent non-specific polymer-polymer interactions. A fluorescent polymer dye was conjugated to a quenching moiety in order to form a quenched polymer with reduced or eliminated fluorescence. Adding the quenched polymer to a mixture of two or more polymer dye conjugates was found to reduce or eliminate non-specific polymer-polymer interactions.

Various violet-excitable fluorescent polymer dyes were conjugated to various quencher moieties. About 15 different quenched polymers were prepared from the violet-excitable fluorescent polymer dyes and about a 10 to 15-fold molar excess of various commercially available quencher moieties by methods analogous to those of the disclosure. Three different violet-excitable fluorescent polymer comprising a structure according to the disclosure (Polymer 1, Polymer 2, Polymer 3) were selected from a range of different MW from 80-150 kDa, or from 90-120 kDa. Commercially available quenching moieties included Dabcyl Q, Dabcyl plus, Anaspec 490Q, Dylight 425Q, Dyomics 425Q, and Dyomics 505Q. Exemplary quenched polymers with quantum yield are shown in Table 2.

TABLE 2
Quantum yield (QY) of polymers with
or without quenching moieties

| | Quenching | Initial molar | Final Q/P* | |
|---|---|---|---|---|
| Polymer Dye | moiety | Q/P ratio | ratio | QY |
| Polymer 2 | DY425Q | 15 | 10.3 | 0.01 |
| Polymer 3 | DY425Q | 15 | 11.2 | 0.009 |
| Polymer 1 | Dabcyl | 10 | 5.6 | 0.01 |
| Polymer 2 | Dabcyl | 10 | 6.8 | 0.005 |
| Polymer 3 | Dabcyl | 10 | 7.1 | 0.005 |
| Polymer 3 | Dabcyl plus | 10 | 5.4 | 0.015 |
| Polymer 3 | None | NA | NA | 0.54 |

*Q/P = quenching moiety/polymer

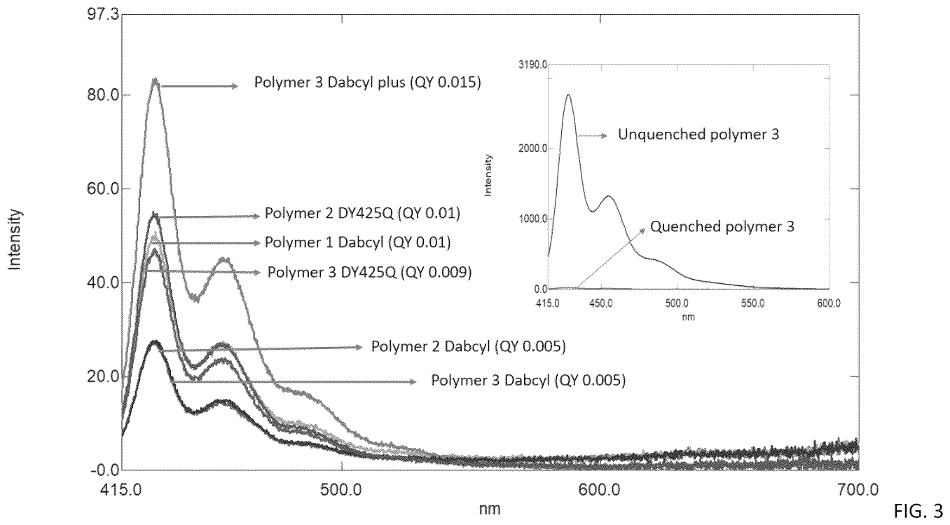

FIG. 3 shows a graph of fluorescent profiles in wavelength (nm) vs. AFU of Quenched polymers: Polymer 1 Dabcyl (QY 0.01), polymer 2 Dabcyl (QY 0.005), polymer 2 DY425Q (QY 0.01), polymer 3 Dabcyl (QY 0.005), polymer 3 Dabcyl plus (QY 0.015), and polymer 3 DY425Q (QY 0.009). FIG. 3 inset shows a graph of representative parent fluorescent polymer (Polymer 3 QY 0.54) before and after conjugation to the quenching moiety to obtain the quenched polymer 3 which exhibits substantially reduced fluorescence QY when excited by a 405 nm laser. QY refers to quantum yield. The quenched polymers in Table 2 each exhibit <0.02, ≤0.015, or ≤0.01 Quantum yield (QY), in contrast to unquenched polymer 3 (QY 0.54) when excited at 405 nm.

### Example 11: Evaluation of Quenched Polymers

The quenched polymers of Table 2 were evaluated using a single dye conjugate or panel of dye conjugates and whole blood samples in FCA assays as follows. The quenched polymers were added at 10 uL of volume (500 ug/mL) for 100 uL of whole blood.

The quenched polymer was used at 5 microgram (5 ug) for 1 conjugate, and 10 micrograms (10 ug) for 2 conjugates in a buffer comprising PBS/BSA/NaN3/PF-68 (if needed). The antibody conjugates (1 ug each) were added on top of the mix of additives before addition of the whole blood. After 20 minutes of incubation, 1 mL of VersaFix was added followed by 15 min of incubation. Finally, a wash with 3 mL of 1×PBS was performed. The pellet was resuspended with 0.5 mL of 1×PBS or 1×PBS/0.1% FA.

The ability of the quenched polymers to decrease non-specific binding and to reduce spread of populations was investigated.

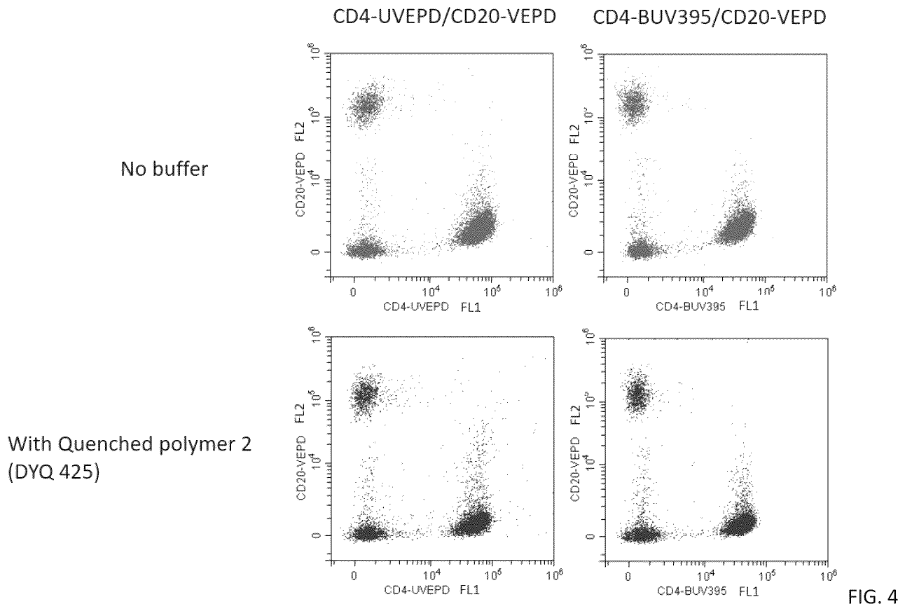

FIG. 4 shows (upper row, left to right) FCA dot plots of stained and lysed whole blood samples after staining with a mixture of the two polymer dye conjugates CD4-UV excitable polymer dye (CD4-UVEPD) and CD20-Violet excitable polymer dye (CD20-VEPD) (both Beckman Coulter Life Sciences, upper left panel), and a mixture of two polymer dye conjugates CD4-BUV395 (BD Biosciences) and CD20-VEPD (Beckman Coulter Life Sciences, upper right panel) when gated on lymphocytes, without staining buffer additive quenched polymer. When two different dye conjugates are mixed without quenched polymer, the dot plots exhibit non-specific interaction and associated spillover. Addition of quenched polymer according to the disclosure (Quenched polymer 2 DYQ 425) during staining with CD4-UVEPD and CD20-VEPD (bottom left panel), or BUV395-CD4 and CD20-VEPD (bottom right panel), reduced non-specific interactions and spillover

Similar improved FCA results were exhibited with test staining buffer comprising quenched polymer 3 DYQ425 (10 uL). (data not shown).

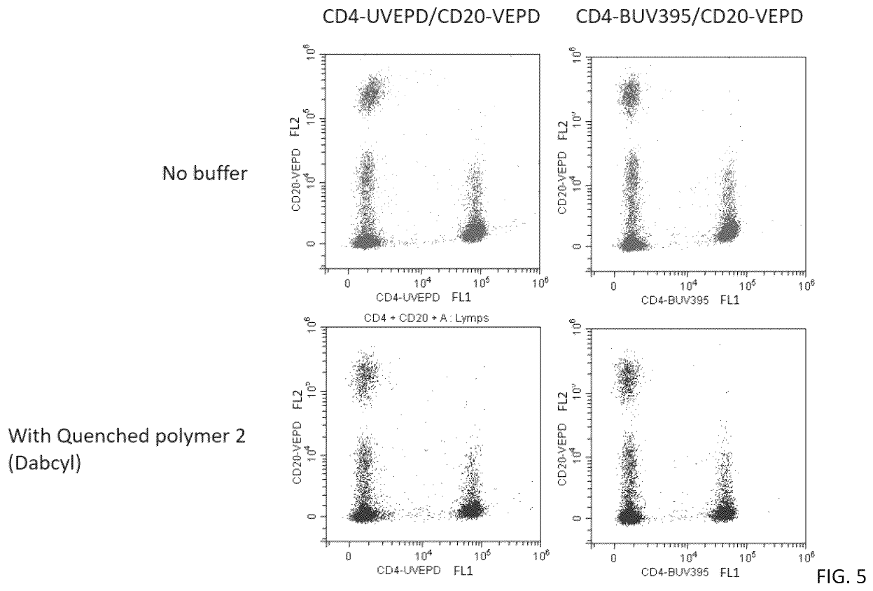

FIG. 5 shows FCA dot plots of stained and lysed whole blood samples after staining without buffer with a mixture of the two polymer dye conjugates CD4-UV excitable polymer dye (CD4-UVEPD) and CD20-Violet excitable polymer dye (CD20-VEPD) (both Beckman Coulter Life Sciences, upper left panel), and with a mixture of two polymer dye conjugates CD4-BUV395 (BD Biosciences) and CD20-VEPD (Beckman Coulter Life Sciences, upper right panel) when gated on lymphocytes, without staining buffer additive quenched polymer. When each of the mixtures of two different dye conjugates are mixed without quenched polymer, the dot plots exhibit non-specific interaction and associated spillover. Addition of quenched polymer according to the disclosure (Quenched Polymer 2-Dabcyl) during staining with CD4-UVEPD and CD20-VEPD (bottom left panel), or BUV395-CD4 and CD20-VEPD (bottom right panel), reduced non-specific interactions and spillover.

Similar improved FCA results were exhibited with other quenched polymers Polymer 1-Dabcyl, Q/P 5.6 (10 uL), Polymer 3-Dabcyl, Q/P 7.14 (10 uL), and Polymer 3-Dabcyl plus, Q/P 5.4 (10 uL). (data not shown). The Polymer-Dabcyl quenched polymers were selected for further development because they exhibited very good ability to reduce spillover, polymer-polymer interaction in polymer dye conjugates.

### Example 12: Exemplary Staining Buffer Composition Comprising a Quenched Polymer

A staining buffer composition was developed using a quenched polymer according to Example 10. As shown in Example 10, the staining buffer Composition B was found to be suitable for reducing spillover, non-specific polymer-polymer interactions in multi-color polymer dye conjugate panels.

PF-68 detergent is added extemporaneously into PBS/BSA/NaN₃ to reach a final concentration of 0.02%. This mix was stored at room temperature until its use to formulate the buffer. The composition is formulated by mixing the components to obtain the composition shown in Table 3.

TABLE 3
Staining Buffer Composition B

| | Final Working | | Concentrated | | |
|---|---|---|---|---|---|
| | Concentration | | Composition | | Amount |
| Component | (1X) | | (10X) | | per test |
| Quenched | 0.3-1.5 | mg/mL | 3-15 | mg/mL | 3-15 |
| polymer | | | | | ug/test |
| PF-68 | 0.02% | (wt/vol) | 0.2% | (wt/vol) | N/A |

| | | | |
|---|---|---|---|
| PBS/BSA/NaN₃ | 1X/2 mg/mL/0.1% | N/A | N/A |

### Example 13: Exemplary Staining Buffer Composition Comprising a Monomeric Component of a Polymer Dye and a Quenched Polymer Dye

A composition was developed comprising exemplary monomer A (a subcomponent of violet 428 SuperNova dye) and a Poly-Dabcyl tandem dye. The Poly-Dabcyl tandem dye was made by conjugating the violet 428 dye monomer to Dabcyl molecules. The Dabcyl is a quencher molecule absorbing the fluorescence emitted by the Violet 428 SuperNova dye when it is excited by the 405 nm laser. In consequence, the Poly-Dabcyl tandem dye doesn't emit much fluorescence.

The general poly dabcyl tandem dye has a structure according to Formula (XXIV). There were approximately 5-8 dabcyl dyes in each polymer backbone.

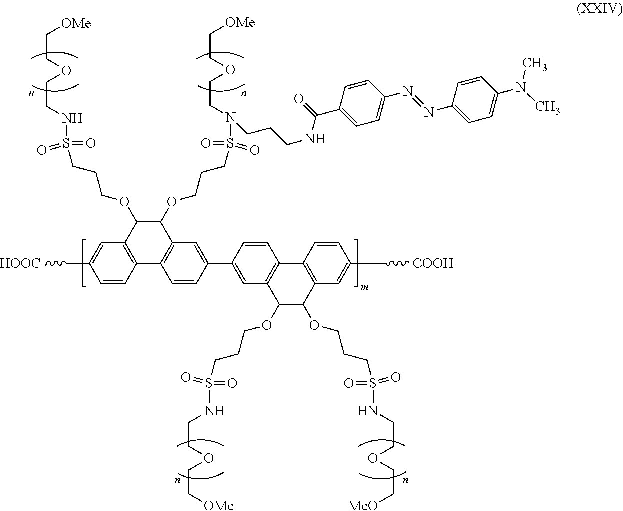

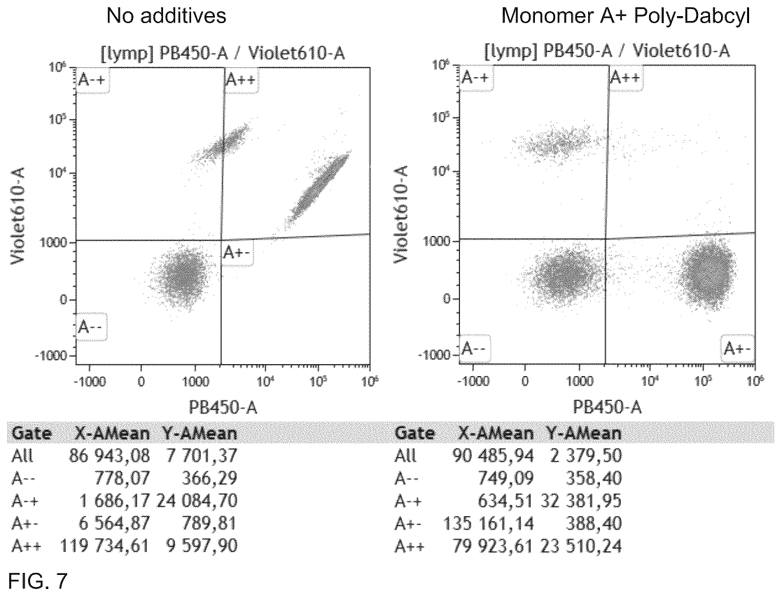

Two SuperNova polymer dye conjugates (CD3-SN428 and CD19-SN605) were individually pre-formulated or not with the exemplary Monomer A +Poly-Dabcyl additives before being added on the biological sample. Buffers and protocols otherwise were similar to Example 10. FIG. 7 shows two-dimensional FCA dot plots without additives (left panel) and with exemplary Monomer A+Poly-Dabcyl additives (right panel) of CD3-SN428/CD19-SN605. In the presence of additives, non-specific interactions of polymer dye conjugates were found to be substantially reduced.

The Poly-Dabcyl violet polymer-dabcyl tandem (XXIV) quenched polymer was found to be efficient in reducing non-specific interactions between different polymer dyes.

PF-68 detergent is added extemporaneously into PBS/BSA/NaN₃ to reach a final concentration of 0.02%. This mix was stored at room temperature until its use to formulate the buffer. The staining buffer composition C is formulated by mixing the components to obtain the composition shown in Table 4.

TABLE 4
Staining Buffer Composition C

| | Final Working | | Concentrated | | |
|---|---|---|---|---|---|
| | Concentration | | composition | | Amount |
| Component | (1X) | | (10X) | | per test |
| Exemplary | 20-40 | mg/mL | 200-400 | mg/mL | 200-400 |
| Monomer A | | | | | ug/test |
| Quenched Polymer | 0.5-1.5 | mg/mL | 5-15 | mg/mL | 5-15 |
| | | | | | ug/test |

| | | | |
|---|---|---|---|
| PF-68 | 0.01-0.05% | 0.1-0.5% | N/A |
| PBS/BSA/NaN₃ | 1X/2 mg/mL/0.1% | N/A | N/A |

### Example 14: Performance of Quenched Polymer Composition With Protein Stabilizer and Nonionic Surfactant

The performance of quenched polymer dye compositions comprising quenched violet Polymer 2-Dabcyl was evaluated in presence and absence of nonionic surfactant Pluronic F-68 (1%) as compared to a commercial BD Horizon™ Brilliant stain buffer Plus. FCA of stained and lysed blood samples from Donor A was performed using a mixture of dye conjugates SuperNova SN v428-CD19 (Beckman Coulter Life Sciences) and BV650-CD4 (Brilliant Violet 650™ anti-human CD4 antibody, BioLegend, Inc.) in a processed blood sample from Donor A.

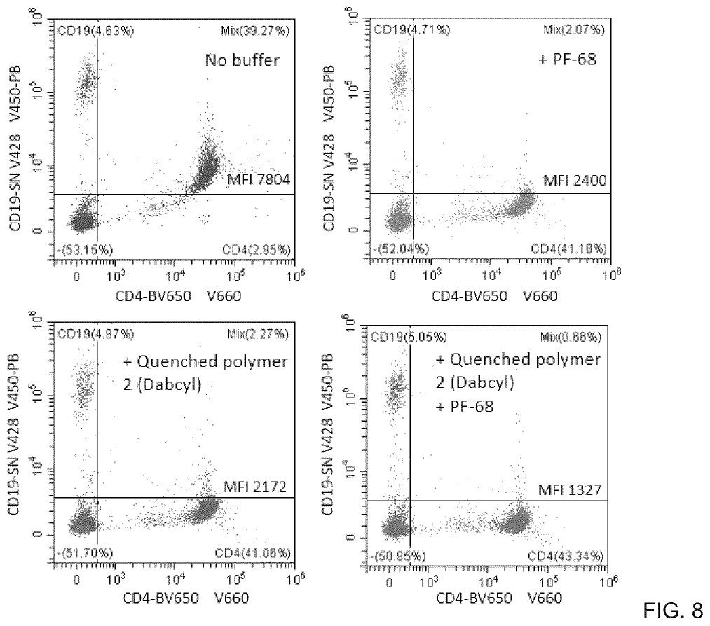

FIG. 8 shows FCA dot plots of a mixture of dye conjugates SuperNova SN v428-CD19 and BV650-CD4 in a blood sample without quenched polymer (upper left), with 1% PF-68 (upper right), with 10 ug quenched polymer 2-Dabcyl (bottom left), with 10 ug quenched polymer 2-Dabcyl and 1% PF-68 nonionic surfactant (bottom right). The test sample compositions with 10 ug quenched polymer 2-Dabcyl (MFI 2172) and 1% PF-68 (MFI 2400) exhibited decreased non-specific binding compared to controlled sample with no buffer (MFI 7804). The test sample composition with 10 ug quenched polymer 2-Dabcyl and 1% PF-68 (MFI 1327) exhibited improved decreased MFI, improved decreased non-specific binding, and improved decreased polymer-polymer interactions compared to controlled samples.

### Example 15. Flow Cytometry Performance of Nonionic Surfactant Alone in Mixtures of Two Different Polymer Dye Conjugates

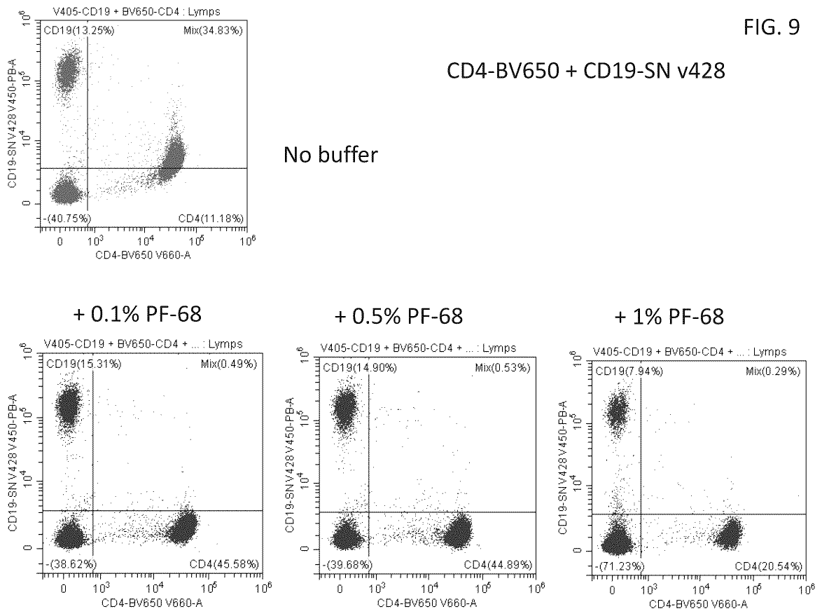

Nonionic surfactant was found to be a desirable additive for reducing non-specific polymer dye conjugate interactions in staining buffer compositions. The effect of different concentrations of nonionic surfactant alone on FCA of stained and lysed blood cells using a mixture of two different polymer dye conjugates was evaluated. FIG. 9 shows FCA dot plots of stained and lysed cells with a mixture of CD4-BV650 (BD Biosciences) and CD19-SNv428 (Beckman Coulter Life Sciences) without buffer (upper panel), with 0.1% PF-68 (lower left panel), 0.5% PF-68 (lower middle panel), and 1% PF-68 (wt/vol) (lower right panel). The presence of increasing concentration of PF-68 (0.1-1% wt/vol) is associated with decreased non-specific interactions in the mixture as evidenced by improved separation compared to without PF-68.

### Example 16. Non-Fluorescent Polymer for Use in Staining Buffer Compositions

Staining buffer compositions comprising non-fluorescent components of polymer dyes including photo-bleached polymer dyes, quenched polymer dyes, and or monomeric components of polymer dyes have been shown to be efficient for reducing spillover and non-specific polymer-polymer interactions in a multi-color panel of two of more polymer dye conjugates in FCA of stained biological samples as demonstrated in the present disclosure.

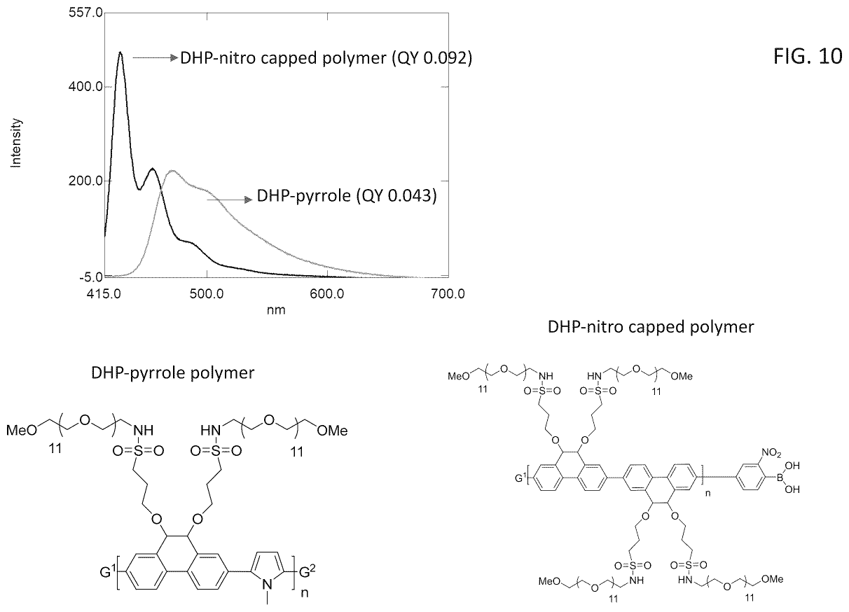

In this example, non-fluorescent polymer dyes having QY of no more than 0.1 were prepared, emission spectra and QY were measured, and tested in staining buffer compositions. FIG. 10 shows a graph of emission spectra over 415-700 nm and quantum yield of two non-fluorescent polymer dyes. Structures of DHP-pyrrole polymer (QY 0.043) and DHP-nitro capped polymer (QY 0.092) are also shown. In FCA studies of lysed and stained cells using at least two polymer dye conjugates, the DHP-pyrrole polymer and DHP-nitro capped polymer were found to be effective for reducing spillover. (data not shown). Non-fluorescent polymer dyes exhibiting a quantum yield (QY) no more than 0.1, or no more than 0.06, or no more than 0.056 were found to be useful for decreasing non-specific interactions and spillover in FCA analysis in staining buffer compositions.

What is claimed is:

1. A composition for use with at least one fluorescent polymer dye conjugated to a binding partner for use in staining a biological sample, the composition comprising:
at least one non-fluorescent component of a first polymer dye, wherein the non-fluorescent component of the first polymer dye is selected from the group consisting of a monomeric component of a polymer dye comprising an aryl moiety or heteroaryl moiety, a photo-bleached polymer dye, a quenched polymer dye, and a non-fluorescent polymer dye;
a nonionic surfactant and
a biological buffer;
wherein the composition reduces non-specific binding of the at least one fluorescent polymer dye conjugate, when compared to the at least one fluorescent polymer dye conjugate in the absence of the composition.

2. The composition of claim 1, wherein the non-fluorescent component of a first polymer dye exhibits at least one of:
a quantum yield (QY) no more than 0.1, no more than 0.06, or no more than 0.056;
less than about 50 arbitrary fluorescence units (AFU) when excited by a 405 nm laser; and
>95% quenching of original maximum emission intensity compared to parent fluorescent polymer when excited at 405 nm.

3. (canceled)

4. The composition according to claim 1, comprising a monomeric component of a polymer dye and a photo-bleached polymer dye.

5. The composition according to claim 1, comprising a monomeric component of a polymer dye and a quenched polymer dye.

6. The composition according to claim 1, wherein the nonionic surfactant is a poloxamer nonionic surfactant.

7. The composition according to claim 1, wherein the at least one polymer dye comprises two or more polymer dyes each conjugated to a binding partner.

8.-16. (canceled)

17. The composition according to claim 1, wherein the aryl moiety or heteroaryl moiety is a dihydrophenanthrene (DHP) moiety, a carbazole moiety, or a fluorene moiety.

18. The composition according to claim 1, wherein the monomeric component of a polymer dye comprises a dihydrophenanthrene (DHP)-based water-soluble monomer having a chemical structure according to Formula (XXII): 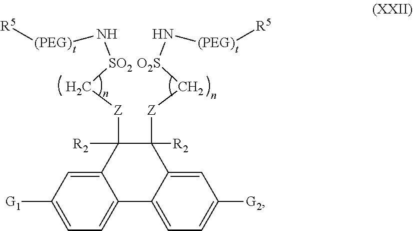
wherein:
each G₁, G₂ is independently selected from the group consisting of halo (F, Cl, Br, I), C₁-C₆ alkyl, and PEG;
each R² is independently selected from the group consisting of a water-solubilizing moiety, alkene, alkyne, cycloalkyl, haloalkyl, (hetero)aryloxy, (hetero)arylamino, sulfonamide-PEG, phosphoramide-PEG, ammonium alkyl salt, ammonium alkyloxy salt, ammonium oligoether salt, sulfonate alkyl salt, sulfonate alkoxy salt, sulfonate oligoether salt, sulfonamido oligoether, sulfonamide, sulfinamide, phosphonamidate, phosphinamide, 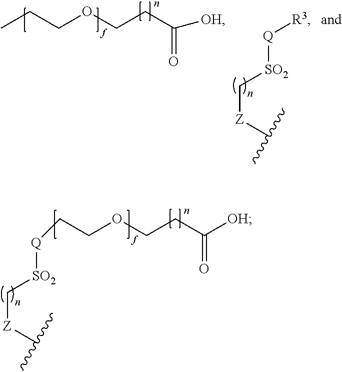
each R³ is a water-solubilizing moiety;
each R⁴ is independently selected from the group consisting of H, alkyl, PEG, a water-solubilizing moiety, a linker moiety, a chromophore, carboxylic amine, amine, carbamate, carboxylic acid, carboxylate ester, maleimide, activated ester, N-hydroxysuccinimindyl, hydrazine, hydrazide, hydrazone, azide, alkyne, aldehyde, or thiol, or protected groups thereof;
each R⁵ is independently selected from the group consisting of H, hydroxyl, C₁-C₁₂ alkyl, C₂-C₁₂ alkene, C₂-C₁₂alkyne, C₃-C₁₂cycloalkyl, C₁-C₁₂ haloalkyl, C₁-C₁₂ alkoxy, C₂-C₁₈ (hetero)aryloxy, C₂-C₁₈ (hetero)arylamino, C₂-C₁₂ carboxylic acid, and C₂-C₁₂ carboxylate ester;
each Q is independently a bond, NR⁴, or —CH₂;
each Z is independently CH₂, O, or NR⁴;
each f is independently an integer from 0 to 50; and
each n is independently an integer from 1 to 20.

19. The composition according to claim 18, wherein G₁ and G₂ are each halo; each Z is O; each R² is H; each n is independently 2-4; and each f is independently 5-20; optionally wherein each n is 3.

20. The composition according to claim 1, wherein the monomeric component of a polymer dye is a fluorene-based or carbazole-based water-soluble monomer having a chemical structure according to Formula (XXIII): 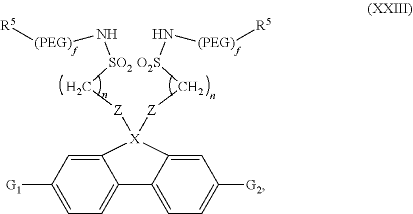
wherein:
each G1, G2 is independently selected from the group consisting of halo (F, Cl, Br, I), C₁-C₆ alkyl, and PEG;
each X is C, N, or Si;
each R⁴ is independently selected from the group consisting of H, alkyl, PEG, a water-solubilizing moiety, a linker moiety, a chromophore, carboxylic amine, amine, carbamate, carboxylic acid, carboxylate ester, maleimide, activated ester, N-hydroxysuccinimidyl, hydrazine, hydrazide, hydrazone, azide, alkyne, aldehyde, or thiol, or protected groups thereof;
each R5 is independently selected from the group consisting of hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, C₂-C₁₂ carboxylic acid, C₂-C₁₂ carboxylate ester, and C₁-C₁₂ alkoxy;
each Z is independently CH₂, O, or NR⁴;
each f is independently an integer from 0 to 50; and
each n is independently an integer from 1 to 20.

21. The composition according to claim 20, wherein G₁ and G₂ are each halo; each Z is O; each R² is H; each n is independently 2-4; and each f is independently 5-20; optionally wherein each n is 3.

22. The composition according to claim 1, wherein the photo-bleached polymer dye is prepared by exposing a fluorescent polymer dye according to formula (I), (VIII), (IX), (X), (XI), (XII), (XIII), (XIV), (XVIII), (XIX), or (XX) to UV light illumination such that it exhibits at least one of:
a quantum yield (QY) no more than 0.1, no more than 0.06, or no more than 0.056;
less than about 50 arbitrary units of fluorescence (AEU), optionally when excited with 405 nm laser; and
>95% quenching of original maximum emission intensity compared to parent fluorescent polymer when excited at 405 nm.

23. The composition according to claim 1, wherein the quenched polymer dye comprises a structure according to any one of Formula (I), (VIII), (IX), (X), (XI), (XII), (XIII), (XIV), (XVIII), (XIX), (XX), or (XXIV), 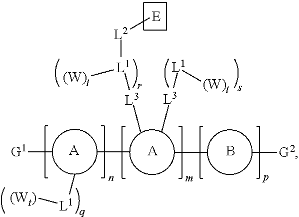
optionally wherein the quenched polymer dye exhibits a fluorescent profile of less than 50 AFU.

24. The composition according to claim 23, wherein the quenched polymer dye comprises a quenching moiety selected from the group consisting of a Dabcyl, Dabsyl, DYQ425, DYQ505, BHQ1, QSY7, QSY9, QSY35, and TAMRA quenching moieties, and optionally wherein m=2-20, 3-15, or 5-8.

25. The composition according to claim 1, wherein the nonionic surfactant is a polyethylene oxide-polypropylene oxide-polyethylene oxide triblock copolymer.

26. The composition according to claim 1, wherein the nonionic surfactant comprises a structure according to formula (XXI) 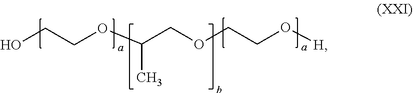
wherein each a is independently in the range of 2-130 and b is in the range of 15-67.

27. (canceled)

28. The composition according to claim 1, further comprising a zwitterionic surfactant.

29. A method for detecting an analyte in a sample comprising:
adding at least one polymer dye conjugate to a composition according to claim 1, to form a polymer dye conjugate composition;
contacting a biological sample that is suspected of containing an analyte with the polymer dye conjugate composition to form a fluorescent polymer dye conjugate complex with the analyte;
applying a light source to the sample that can excite the at least one fluorescent polymer dye conjugate complex; and
detecting light emitted from the fluorescent polymer dye conjugate complex.

30.-38. (canceled)

39. A kit comprising a composition according to claim 1, and at least one fluorescent polymer dye or fluorescent polymer dye conjugate, optionally wherein the composition and the polymer dye or polymer dye conjugate are in separate containers.

40.-42. (canceled)
